(19) United States
(12) Patent Application Publication
(10) Pub. No.: US 2018/0315428 A1
(43) Pub. Date: Nov. 1, 2018

(54) EFFICIENT TRANSCRIPTION SYSTEMS AND METHODS

(71) Applicant: 3Play Media, Inc., Boston, MA (US)

(72) Inventors: Christopher E. Johnson, Belmont, MA (US); Roger S. Zimmerman, Boston, MA (US); Joshua Miller, Charlestown, MA (US); Jeremy E. Barron, Boston, MA (US); Christopher S. Antunes, Boston, MA (US)

(73) Assignee: 3Play Media, Inc., Boston, MA (US)

(21) Appl. No.: 15/963,655

(22) Filed: Apr. 26, 2018

Related U.S. Application Data
(60) Provisional application No. 62/490,768, filed on Apr. 27, 2017.

(51) Int. Cl. G10L 15/26; G10L 15/01; G06F 3/16; G16H 10/60

(52) U.S. Cl. CPC G10L 15/265; G16H 10/60; G06F 3/165; G10L 15/01

(57) ABSTRACT

A mobile computing device implementing a mobile recording application is provided. The mobile computing device comprises a memory, a microphone, a network interface, and a processor. The processor is configured to record, via the microphone, at least one media file comprising content divisible into a plurality of sections; associate a first portion of the at least one media file with a first section of the plurality of sections; associate a second portion of the at least one media file with a second section of the plurality of sections; generate transcription request information specifying that the first portion be transcribed without human review and that the second portion be transcribed with human review; and transmit, via the network interface, the at least one media file and the transcription request information to a transcription system distinct from the mobile computing device.

### RELATED APPLICATIONS

The present application claims priority under 35 U.S.C. § 119(e) to U.S. Provisional Application 62/490,768, filed on Apr. 27, 2017 and titled “EFFICIENT MEDICAL TRANSCRIPTION SYSTEMS AND METHODS”, which is hereby incorporated herein by reference in its entirety. The present application relates to U.S. Pat. No. 9,704,111, issued on Jul. 11, 2017 and titled “ELECTRONIC TRANSCRIPTION JOB MARKET” (“Electronic Transcription Job Market patent”), which is hereby incorporated herein by reference in its entirety. The present application relates to U.S. Pat. No. 8,930,308, issued on Jan. 6, 2015 and titled “METHODS AND SYSTEMS OF ASSOCIATING METADATA WITH MEDIA” (“Metadata Media Associator patent”), which is hereby incorporated herein by reference in its entirety.

### NOTICE OF MATERIAL SUBJECT TO COPYRIGHT PROTECTION

Portions of the material in this patent document are subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### Technical Field

The technical field of the present disclosure relates generally to transcription of content and, more particularly, to systems and methods that efficiently transcribe and organize divisible content recorded under a variety of environmental conditions.

### Discussion

Electronic Health Record (EHR) systems have been widely adopted by doctors in the United States. This adoption has ostensibly been driven by both cost and revenue incentives, such as increased operational efficiency and Medicaid/Medicare reimbursement requirements. However, as a practical matter, wide-spread adoption of EHR systems has resulted in doctors devoting substantial amounts of time toward accurately documenting patient encounters within appropriate sections of the electronic record.

One way that doctors have traditionally saved time on such documentation is by verbally dictating notes that are transcribed by medical transcriptionists. Doctors who use medical transcription services record a dictation or speak through a landline to provide a recording to a medical transcriptionist who is either in-house or part of a third-party service. Once a transcript is complete, the doctor can review, copy and paste portions of the transcript into an Electronic Health Record (EHR) to convey the details of a patient encounter.

However, medical transcription services are costly, on the order of 12 to 15 cents per line of transcription. While the increasing accuracy of Automatic Speech Recognition (ASR) systems has improved some transcription processes, ASR systems are not robust enough for many applications outside a quiet environment and in which the user can speak very clearly. Even though the cost of automatic transcription is less than full transcription services, the cost to review, edit and manage the results of speech recognition can outweigh the initial benefit.

In recent years, to save time, doctors have tried a variety of solutions, including the employment of medical scribes, who follow the doctors and furiously type what is happening during a patient encounter to complete the medical record as it is happening in real time. However, this approach is disruptive to the patient experience, and for many doctors, is cost-prohibitive.

### SUMMARY

EHRs exemplify a broader class of divisible content that is recorded under a variety of environmental conditions and for which transcripts are organized into standardized sections for on-line retrieval and review. Thus, while the conventional techniques described above for EHRs are also applicable to divisible content, the conventional techniques suffer from the same challenges described above when applied to this broader class of content.

Thus, and in accordance with at least some embodiments described herein, systems and methods are provided for efficiently transcribing divisible content recorded under a variety of environmental conditions (e.g., quiet environments, noisy environments, environments disparately located temporally or spatially from one another, etc.). These system and methods leverage, to advantageous effect, differences in recording quality of the divisible content that result from these varying environmental conditions. For instance, some embodiments perform additional processing to sections of the divisible content only where such additional processing is needed to ensure a quality transcript (e.g., where the sections were recorded in a noisy environment). By avoiding the additional processing where it is not required (e.g., for sections recorded in a quiet environment), these embodiments process the divisible content more efficiently than conventional techniques, which subject all sections of the divisible content to the same level of processing. While the systems and method described herein focus on EHR systems and methods as one particular example, it is appreciated that the systems and methods disclosed herein are applicable to any divisible content that is recorded under varying environmental conditions and for which transcripts are divided into standardized sections that are stored within a database for subsequent retrieval and review.

In at least one embodiment, the systems and methods disclosed herein are configured to save doctors time when generating EHR entries documenting patient encounters. In some embodiments, the systems and methods include and utilize a mobile recording application executing on a mobile computing device, such as a smart phone, laptop, or personal digital assistant. The mobile recording application is configured to present a user interface that is tailored to efficient generation of EHR entries. This user interface may include visual, audio, and tactile elements, which are described further below. The mobile recording application may also implement one or more of a variety features designed to increase efficiency in adding patient encounters to the EHR. Moreover, the user interface includes screens that enable health care providers to efficiently scan and review historical patient encounters that are documented within the EHR.

In some embodiments, the mobile recording application is configured to record audio entries uttered by doctors via a microphone included in the mobile computing device and to associate the recorded audio entries with particular sections of the EHR. In some of these embodiments, the mobile recording application associates audio entries with sections in response to receiving user input indicating the association. For example, the mobile recording application may receive user input requesting that the mobile recording application begin recording a particular EHR section and, in response, the mobile recording application may being recording and may store an association between the recording and the particular EHR section. In other embodiments, the mobile recording application searches the audio entries for keywords associated with the sections and associates audio entries including (e.g., starting with) the keywords with their associated sections. This flexible approach to associating audio entries with EHR sections promotes freedom and flexibility in recording audio entries, which in turn enhanced dictation productivity.

In other embodiments, the mobile recording application is configured to transcribe various audio entries to selected levels of quality prior to porting the audio entries to the EHR. In these embodiments, the mobile recording application is configured to interoperate with, or be incorporated in, a distributed transcription system, such as the transcription system 100 described within Electronic Transcription Job Market patent. The mobile recording application is configured to transmit one or more audio entries to the transcription system and the transcription system, in turn, is configured to automatically transcribe the audio entries to the selected level of quality. This level of quality may be affected by whether and to what extent humans review automatically generated transcripts.

In certain embodiments, the user interface presented by the mobile recording application includes interactive transcript review screens. These screens enable a health care provider to interact with transcript text and audio entries to further refine the EHR. Further, in some embodiments, the mobile recording application and/or the transcription system transmits final transcripts of patient encounters to an EHR for importation. The final transcripts may be segmented into EHR sections to facilitate incorporation of the transcripts into the EHR system.

In some embodiments, the mobile recording application supports voice macros. Voice macros enable health care providers to create standardized, short sets of trigger text that, when identified during transcription, are expanded into longer sets of expansion text. Voice macros can save health care providers substantial time with dictating audio entries into the EHR.

In one embodiment, a mobile computing device is provided. The mobile computing device implements a mobile recording application. The mobile computing device comprises a memory, a microphone, a network interface, and at least one processor coupled to the memory, the microphone, and the network interface. The at least one processor is configured to record, via the microphone, at least one media file comprising content divisible into a plurality of sections; associate a first portion of the at least one media file with a first section of the plurality of sections; associate a second portion of the at least one media file with a second section of the plurality of sections; generate transcription request information specifying that the first portion be transcribed without human review and that the second portion be transcribed with human review; and transmit, via the network interface, the at least one media file and the transcription request information to a transcription system distinct from the mobile computing device.

In the mobile computing device, the content may be descriptive of a patient encounter to be documented in an electronic health record (EHR) of the patient and the plurality of sections may include EHR sections. The at least one processor may be configured to associate the first portion of the at least one media file with the first section in response to identifying a keyword within the first portion, the keyword being associated with the first section. The mobile computing device may further include a display configured to present at least one control associated with the first section. The at least one processor may be coupled to the display and configured to associate the first portion of the at least one media file with the first section in response to receiving a selection of the at least one control prior to recording the first portion. The mobile computing device may further include a display configured to present a plurality of controls comprising a first control associated with the first section and a second control associated with the second section. The at least one processor may be configured to generate the transcription request information at least in part by identifying that the first control is deselected and identifying that the second control is selected.

In the mobile computing device, the at least one processor may be further configured to deselect the first control and select the second control in response to accessing information representative of a default set of sections. The at least one processor may be further configured to deselect the first control in response to a first selection received via the display. The at least one processor may be further configured to initiate generation an automatic speech recognition (ASR) transcript of at least the first portion of the at least one media file; compare an indicator of confidence in the ASR transcript to a threshold confidence; and select the first portion to be transcribed without human review in response to the indictor being greater than the threshold confidence. The at least one processor is further configured to initiate generation an automatic speech recognition (ASR) transcript of at least the second portion of the at least one media file; compare an indicator of confidence in the ASR transcript to a threshold confidence; and select the second portion to be transcribed with human review in response to the indictor being less than the threshold confidence. The at least one processor is configured to initiate generation of the ASR transcript by either initiating a local ASR process or transmitting a message to an ASR system distinct from the mobile computing device.

In another embodiment, a transcript delivery system is provided. The transcript delivery system includes a mobile computing device and a transcription system distinct from the mobile computing device. The mobile computing device implements a mobile recording application. The mobile computing device includes a memory, a microphone, a network interface, and at least one processor coupled to the memory, the microphone, and the network interface. The at least one processor is configured to record, via the microphone, at least one media file comprising content divisible into a plurality of sections; associate a first portion of the at least one media file with a first section of the plurality of sections; associate a second portion of the at least one media file with a second section of the plurality of sections; generate transcription request information specifying that the first portion be transcribed without human review and that the second portion be transcribed with human review; and transmit, via the network interface, the at least one media file and the transcription request information to a transcription system distinct from the mobile computing device. The transcription system is configured to generate a final transcript of the at least one media file in response to receiving the at least one media file and the transcription request information; and transmit the final transcript to a database system distinct from the transcript delivery system.

In the transcript delivery system, the content may be descriptive of a patient encounter to be documented in an electronic health record (EHR) of the patient, the plurality of sections may include EHR sections, and the final transcript may be divided into the EHR sections.

In another embodiment, a method of efficiently transcribing content divisible into a plurality of sections is provided. The method is implemented using a computer system comprising a mobile computing device. The method comprises acts of recording, via a microphone of the mobile computing device, at least one media file comprising the content; associating a first portion of the at least one media file with a first section of the plurality of sections; associating a second portion of the at least one media file with a second section of the plurality of sections; generating transcription request information specifying that the first portion be transcribed without human review and that the second portion be transcribed with human review; and transmitting, via a network interface of the mobile computing device, the at least one media file and the transcription request information to a transcription system distinct from the mobile computing device.

In the method, the act of recording the at least one media file may include an act of recording content descriptive of a patient encounter to be documented in an electronic health record (EHR) of the patient, the content being divisible into EHR sections. The act of associating the first portion of the at least one media file with the first section may include an act of identifying a keyword within the first portion, the keyword being associated with the first section. The method may further include an act of presenting, via a display of the mobile computing device, at least one control associated with the first section, wherein associating the first portion of the at least one media file with the first section comprises receiving a selection of the at least one control prior to recording the first portion. The method may further include an act of presenting, via a display of the mobile computing device, a plurality of controls including a first control associated with the first section and a second control associated with the second section, wherein generating the transcription request information comprises identifying that the first control is deselected and identifying that the second control is selected. The method may further include acts of deselecting the first control and selecting the second control in response to accessing information representative of a default set of sections. The method may further include an act of deselecting the first control in response to a first selection received via the display.

The method may further include acts of initiating generation an automatic speech recognition (ASR) transcript of at least the first portion of the at least one media file; comparing an indicator of confidence in the ASR transcript to a threshold confidence; and selecting the first portion to be transcribed without human review in response to the indictor being greater than the threshold confidence. The method may further include acts of initiating generation an automatic speech recognition (ASR) transcript of at least the second portion of the at least one media file; comparing an indicator of confidence in the ASR transcript to a threshold confidence; and selecting the second portion to be transcribed with human review in response to the indictor being less than the threshold confidence.

In the method, the act of initiating generation of the ASR transcript may include either an act of initiating a local ASR process or an act of transmitting a message to an ASR system distinct from the mobile computing device. The method may further include acts of generating, by a transcription system distinct from the mobile computing device, a final transcript of the at least one media file in response to receiving the at least one media file and the transcription request information; and transmitting the final transcript to a database system distinct from the transcript delivery system. In the method, the act of generating the final transcript may include an act of generating a final transcript of a patient encounter to be documented in an electronic health record (EHR) of the patient, the final transcript being divided into EHR sections.

In another embodiment, a non-transitory computer readable medium storing sequences of computer executable instructions for efficiently transcribing content divisible into a plurality of sections is provided. The sequences of computer executable instructions include instructions that instruct at least one processor to recording, via a microphone of the mobile computing device, at least one media file comprising the content; associating a first portion of the at least one media file with a first section of the plurality of sections; associating a second portion of the at least one media file with a second section of the plurality of sections; generating transcription request information specifying that the first portion be transcribed without human review and that the second portion be transcribed with human review; and transmitting, via a network interface of the mobile computing device, the at least one media file and the transcription request information to a transcription system distinct from the mobile computing device.

In the computer readable medium, recording the at least one media file may include recording content descriptive of a patient encounter to be documented in an electronic health record (EHR) of the patient, the content being divisible into EHR sections.

In another embodiment, a system is provided. The system includes a mobile computing device and a transcription system. The mobile computing device implements a mobile application. The mobile computing device comprises a memory, a microphone, a network interface, and at least one processor coupled to the memory, the microphone, and the network interface. The at least one processor is configured to record, via the microphone, audio comprises a plurality of electronic health record (EHR) sections; identify a first EHR section of the plurality of EHR sections within the audio; identify a second EHR section of the plurality of EHR sections within the audio; generate an order specifying that the first EHR section be transcribed via automatic speech recognition only and that the second EHR section be reviewed by a professional transcription editor; and transmit the audio and the order to a transcription system distinct from the mobile computing device. The transcription system is configured to generate a final transcript of the audio in response to receiving the audio and order; and post the final transcript to an EHR system distinct from the mobile computing device and the transcription system.

The embodiments described herein provide several benefits over conventional medical transcription systems and methods. For example, the ability to select quality levels makes some embodiments robust to noisy environments, thus providing health care providers flexibility with regard to the environments in which they record audio entries. In addition, the ability to select a quality level for audio entry transcription provides cost flexibility to doctors in that automatic transcriptions of high quality need not be the subject of costly human labor. Moreover, random access to particular sections of the EHR enables doctors to record or review audio entries in an organized fashion.

Still other aspects, embodiments and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Any embodiment disclosed herein may be combined with any other embodiment. References to “an embodiment,” “an example,” “some embodiments,” “some examples,” “an alternate embodiment,” “various embodiments,” “one embodiment,” “at least one embodiment,” “this and other embodiments” or the like are not necessarily mutually exclusive and are intended to indicate that a particular feature, structure, or characteristic described in connection with the embodiment may be included in at least one embodiment. The appearances of such terms herein are not necessarily all referring to the same embodiment.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Various aspects of at least one embodiment are discussed below with reference to the accompanying figures, which are not intended to be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure. In the figures:

FIG. 1 is a schematic diagram of a mobile computing device configured in accordance with at one embodiment disclosed herein.

FIG. 2 is a schematic diagram of a mobile recording application configured in accordance with at one embodiment disclosed herein.

FIG. 3 is an illustration of a home screen configured in accordance with at one embodiment disclosed herein.

FIG. 4 is a flow diagram illustrating an interface process in accordance with at one embodiment disclosed herein.

FIG. 5 is an illustration of an appointments screen configured in accordance with at one embodiment disclosed herein.

FIG. 6 is a flow diagram illustrating another interface process in accordance with at one embodiment disclosed herein.

FIG. 7 is an illustration of a recording screen configured in accordance with at one embodiment disclosed herein.

FIG. 8 is an illustration of another recording screen configured in accordance with at one embodiment disclosed herein.

FIG. 9 is a flow diagram illustrating another interface process in accordance with at one embodiment disclosed herein.

FIG. 10 is an illustration of a transcription ordering screen configured in accordance with at one embodiment disclosed herein.

FIG. 11 is a flow diagram illustrating another interface process in accordance with at one embodiment disclosed herein.

FIG. 12 is an illustration of a patient search screen configured in accordance with at one embodiment disclosed herein.

FIG. 13 is a flow diagram illustrating another interface process in accordance with at one embodiment disclosed herein.

FIG. 14 is an illustration of a patient transcripts screen configured in accordance with at one embodiment disclosed herein.

FIG. 15 is an illustration of another patient transcripts screen configured in accordance with at one embodiment disclosed herein.

FIG. 16 is an illustration of a transcript screen configured in accordance with at one embodiment disclosed herein.

FIG. 17 is a flow diagram illustrating another interface process in accordance with at one embodiment disclosed herein.

FIG. 18 is an illustration of a keyword search screen configured in accordance with at one embodiment disclosed herein.

FIG. 19 is a flow diagram illustrating another interface process in accordance with at one embodiment disclosed herein.

FIG. 20 is an illustration of a transcript defaults screen configured in accordance with at one embodiment disclosed herein.

FIG. 21 is a flow diagram illustrating another interface process in accordance with at one embodiment disclosed herein.

FIG. 22 is a context diagram including an exemplary transcription system in accordance with at one embodiment disclosed herein.

FIG. 23 is a schematic diagram of the server computer shown in FIG. 22 in accordance with at one embodiment disclosed herein.

FIG. 24 is a schematic diagram of one example of a computer system in accordance with at one embodiment disclosed herein.

FIG. 25 is a flow diagram illustrating a process for creating a transcription job in accordance with at one embodiment disclosed herein.

FIG. 26 is an illustration of a voice macro screen in accordance with at one embodiment disclosed herein.

FIG. 27 is an illustration of a voice macro edit screen in accordance with at one embodiment disclosed herein.

FIG. 28 is an illustration of a preview screen in accordance with at one embodiment disclosed herein.

FIG. 29 is an illustration of an edit screen in accordance with at one embodiment disclosed herein.

FIG. 30 is a flow diagram illustrating a process for editing a transcription job in accordance with at one embodiment disclosed herein.

FIG. 31 is a flow diagram illustrating a process for calibrating a job in accordance with at one embodiment disclosed herein.

FIG. 32 is a flow diagram illustrating a process for determining transcription job attributes in accordance with at one embodiment disclosed herein.

FIG. 33 is a flow diagram illustrating states assumed by a transcription job during execution of an exemplary transcription system in accordance with at one embodiment disclosed herein.

FIG. 34 is an illustration of another recording screen configured in accordance with at one embodiment disclosed herein.

### DETAILED DESCRIPTION

At least one embodiment disclosed herein includes apparatus and processes configured to implement, via a mobile computing device, a mobile recording application. This mobile recording application is tailored to increase the efficiency of a health care provider in documenting patient encounters within the EHR. This mobile recording application may alternatively be configured to increase the efficiency of a user dictating audio for the purpose of adding textual records to a database.

Examples of the methods and systems discussed herein are not limited in application to the details of construction and the arrangement of components set forth in the following description or illustrated in the accompanying drawings. The methods and systems are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated references is supplementary to that of this document; for irreconcilable inconsistencies, the term usage in this document controls.

### Mobile Recording Application

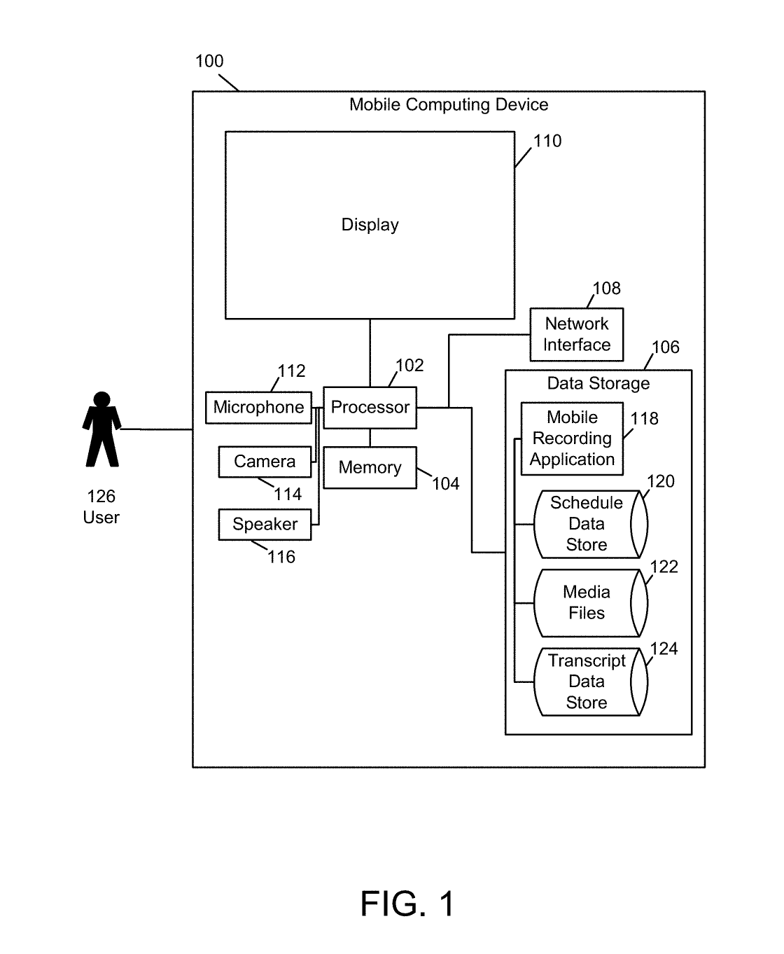

Various embodiments implement a mobile recording application using a computer system, such as a mobile computing device. FIG. 1 illustrates one of these embodiments, a mobile computing device 100 configured to implement a mobile recording application 116. As shown, FIG. 1 includes the mobile computing device 100 and a user 126. The user 126 may be a health care provider (e.g., a doctor, physician assistant, nurse practitioner, or other caregiver who contributes to the EHR of a patient) or some other user who dictates divisible content. The mobile computing device 100 is associated with and used by the user 126. The mobile computing device 100 may be a smart phone, personal digital assistant, laptop, tablet, or any other mobile computer system. The mobile computing device 100 and the mobile recording application 116 may also be used by any user 126 dictating with the intent of creating a transcript of the dictation that is to be inserted into a database.

As shown in FIG. 1, the mobile computing device 100 includes a processor 102, a memory 104, data storage 106, a network interface 108, a display 110, a microphone 112, a camera 114, and a speaker 116. In some embodiments, the processor 102 is configured to implement the mobile recording application by executing a series of instructions that result in manipulated data. The processor 102 may be any type of processor, multiprocessor or controller. Some example processors include commercially available processors such as the ARM Cortex A8 or the Apple A11.

The memory 104 is configured to store programs and data during operation of the mobile computing device 100. The memory 104 may be a relatively high performance, volatile, random access memory such as a dynamic random access memory (DRAM) or static memory (SRAM). However, the memory 104 may include any device for storing data, such as a disk drive or other non-volatile storage device. Various examples may organize the memory 104 into particularized and, in some cases, unique structures to perform the functions disclosed herein. These data structures may be sized and organized to store values for particular data and types of data.

The data storage 106 is configured to store data for extended periods of time, regardless of whether power is supplied to the mobile computing device 100. The data storage may include a computer readable and writeable nonvolatile, or non-transitory, data storage medium in which instructions are stored that define programs or other objects that are executable by the processor 102. The data storage 106 also may store information that is recorded, on or in, the medium, and that is processed by the processor 102 during execution of the program. More specifically, the information may be stored in one or more data structures specifically configured to conserve storage space or increase data exchange performance. The instructions may be persistently stored as encoded signals, and the instructions may cause the processor 102 to implement one or more of the features described herein. The medium may, for example, be optical disk, magnetic disk or flash memory, among others.

The network interface 108 is configured to exchange (e.g., transmit and/or receive) data with other computing devices. The network interface 108 may include an antenna configured to exchange data wirelessly and/or a physical connector configured to exchange data over a cable or other wire.

The display 110 is configured to emit light to render visual elements for presentation. In some embodiments, the display 104 includes a touchscreen configured to detect tactile input via, for example, a change in resistance or capacitance.

The microphone 112 is configured to detect sound present in the ambient environment, which may include, of example, utterances vocalized by the user 126. These utterances may include audio entries for the EHR. The microphone 112 may include, for example, a transducer that converts acoustic signals into electric signals. In some embodiments, the microphone 112 is configured to record audio entries in an environment where the speaker concurrently performs multiple tasks. As such, the microphone 112 may be configured to filter background noise to increase the quality of the recording as the user 126 moves about the environment and/or manipulates objects other than the mobile computing device 100.

The camera 114 is configured to detect light and to store representations thereof in memory (e.g., onboard memory or the memory 104) for subsequent processing. These representations may include arrays of pixel values specifying colors. The camera 108 may include a lens and an array of light detectors to generate the pixel values.

The speaker 116 is configured to generate audio output, which may include playback of vocal utterances previously recorded by the user 126. The speaker may include, for example, a transducer that mechanically converts electric signals into acoustic signals.

The components of the mobile computing device 100 described above are communicatively coupled to one another by interconnection circuitry, such as interconnects, system buses, memory controllers, northbridges, southbridges, and the like. This interconnection circuitry enables communications, such as data and instructions, to be exchanged between these components. Further, the interconnection circuitry enables the processor 102 to control the operation of the remaining components.

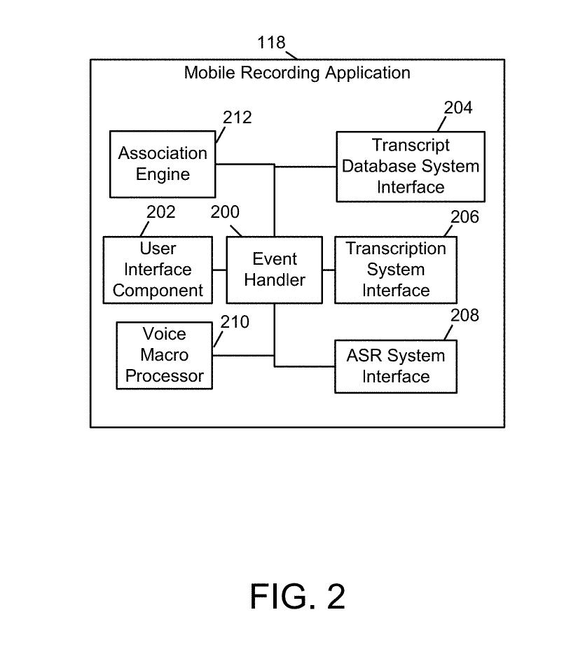

As shown in FIG. 1, the data storage 106 persistently stores a mobile recording application 118, schedule data store 120, media files 122, and transcript data store 124. The mobile recording application 118 includes encoded instructions that are executable by the processor 102 to implement various features described below with reference to FIGS. 2-21. Thus, as illustrated in FIG. 1, the mobile recording application 118 is a software component. However, in other embodiments, the mobile recording application 118 is a hardware component or a combination of hardware and software components that is executable by the processor 102 to implement the various features of the mobile recording application 118 described herein.

The schedule data store 120 is a data structure populated with information regarding patient appointments for the healthcare provider 126. This information may include patient names, dates and times of appointments, indicators of patient locations, and indicators of a degree of completion of the EHR for the appointment.

The media files 122 are data structures populated with content recorded by the mobile recording application 118 via the microphone 112 and/or the camera 114. For instance, each of the media files may contain one or more audio entries for the EHR of a patient. The media files may be recorded in any of a variety of formats, such as .wav, .mp3, .mov, or the like.

The transcript data store 124 is a data structure populated with transcripts of previously generated EHR entries. As such, the transcript data may include textual content that is associated (e.g., via a time or frame index) with a previous audio entry pertinent to a patient. The transcript data store 124 may also include other metadata (e.g., an inverse index or other search structure) that can facilitate searching of the previously generate EHR entries.

In some embodiments, the data storage 106 also persistently stores an operating system that is executable by the processor 102 to provide application programs, such as the mobile recording application 118, with a functional computing environment that is abstracted from the various hardware components described above. In these embodiments, the operating system controls operation of the various hardware components and exposes their functions to application programs via hooks, system calls, and other interface mechanisms. Through these interface mechanisms of the operating system, the application programs can exchange messages with the hardware components to implement specific features, such as the features of the mobile recording application 118 as described herein.

Information within the mobile computing device 100, including data within the schedule data store 120, the media files 122, and the transcript data store 124, may be stored in any logical construction capable of holding information on a computer readable medium including, among other structures, file systems, flat files, indexed files, hierarchical databases, relational databases, and object oriented databases. The data may be modeled using unique and foreign key relationships and indexes. The unique and foreign key relationships and indexes may be established between the various fields and tables to ensure both data integrity and data interchange performance.

In some embodiments, the mobile recording application 118 includes various components that interoperate to execute its features. FIG. 2 illustrates one of these embodiments. As shown in FIG. 2, the mobile recording application 118 includes an event handler 200, a user interface component 202, a transcript database system interface 204, a transcription system interface 206, an ASR system interface 208, a voice macro processor 210, and an association engine 212.

The event handler 200 is configured to process messages received via the operating system of the mobile computing device 100. These messages may include messages indicating user input from the display 110, the microphone 112, and/or the camera 114. The messages may also include messages from other processes executing on the mobile computing device 100 or on other computing devices (e.g., messages received via the network interface 108). The messages may also include housekeeping messages such as acknowledgments and confirmations of successfully executed operations (e.g., successful receipt of a message, successful storage of date, etc.).

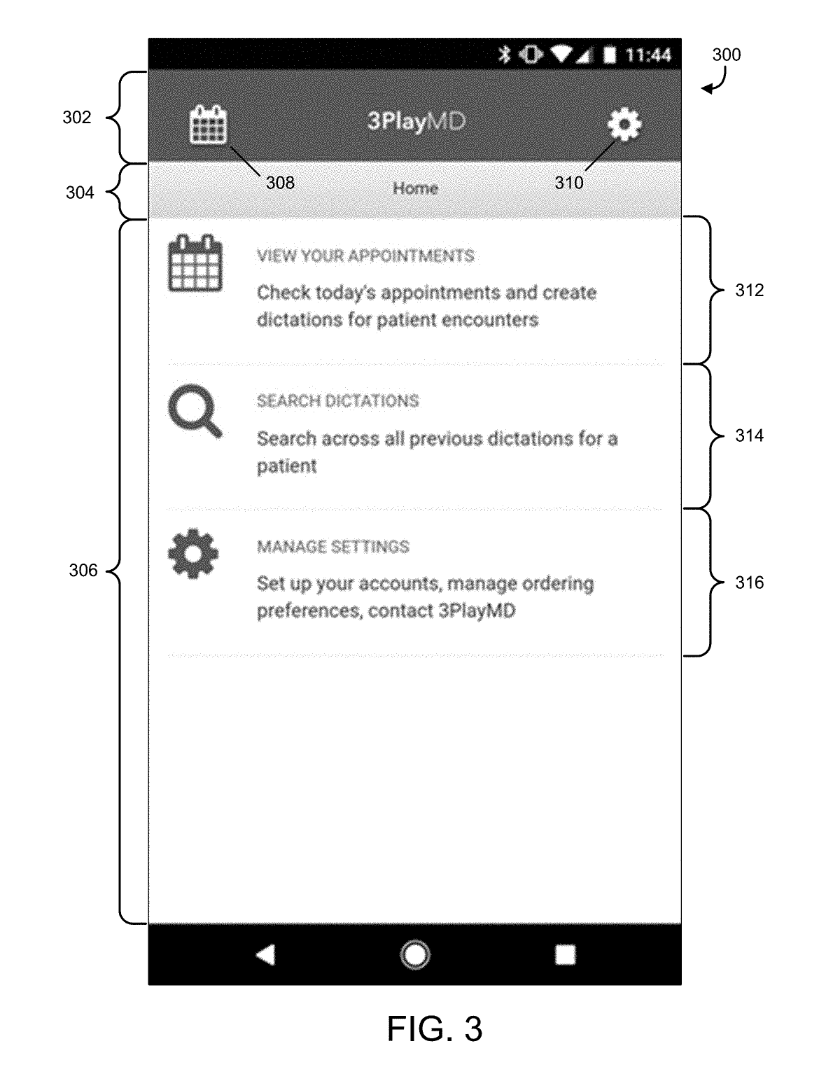

In some embodiments, the event handler 200 is configured to process messages originating from or addressed to a user (e.g., the user 126) by passing them to the user interface component 202. In these embodiments, the user interface component 202 is configured to control the operation of the display 110, the microphone 112, the camera 114 and/or the speaker 116 to implement the various user facing features of the mobile recording application 118. These user facing features are described further below with reference to FIGS. 3-21.

In some embodiments, the event handler 200 is configured to process messages originating from or addressed to a transcript database system (e.g., an EHR system) by passing them to the transcript database system interface 204. In these embodiments, the transcript database system interface 204 is configured to exchange messages with a remote transcript database system via an application program interface (API) exposed by the transcript database system. The transcript database system interface 204 can thereby transmit information, such as EHR entries documenting a patient encounter, to the transcript database system and/or receive information, such as transcripts documenting previous patent encounters within the EHR, from the transcript database system. Examples of EHR systems that the transcript database system interface 204 is configured to exchange messages with include EHR systems provided by AthenaHealth, Epic Systems, Allscripts, eClinicalWorks, and Cerner. More generally, at least some embodiments of the transcript database system interface 204 can exchange information with any text storage database, such as a MySQL database, an Oracle database, a MongoDB database, or a Redis Database, or any web application connected to a text storage database.

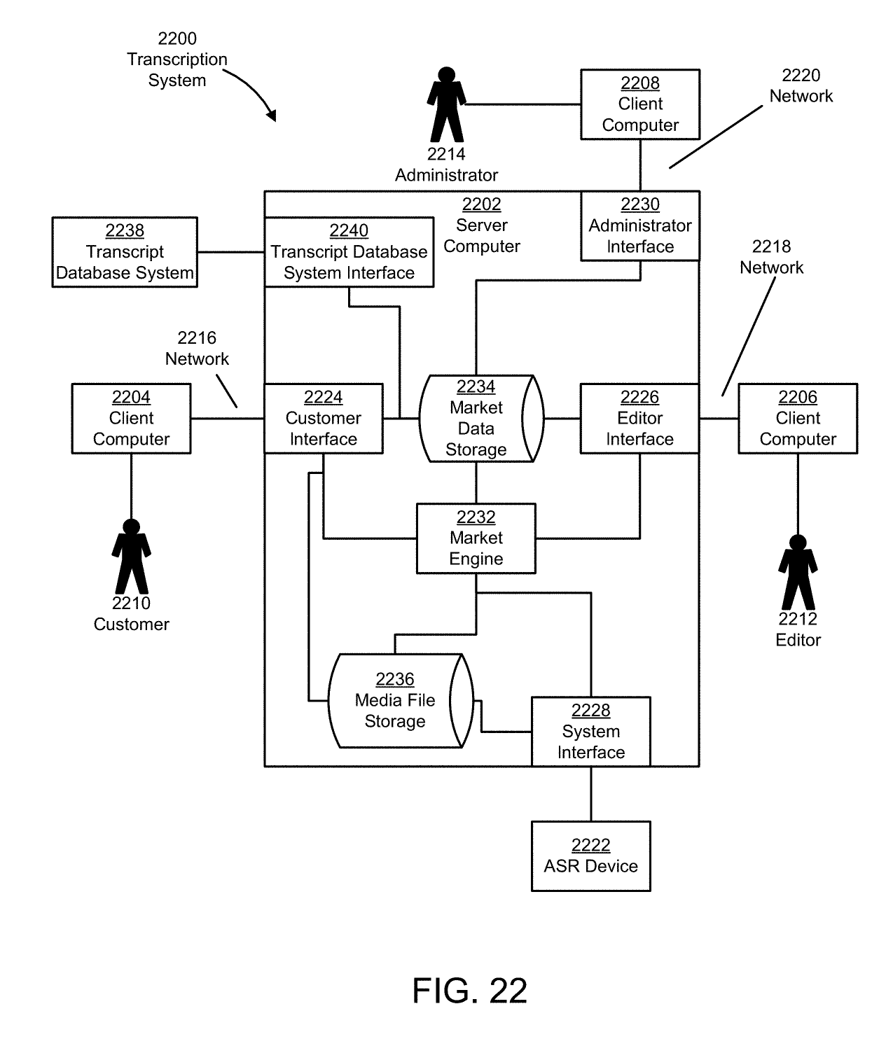

In some embodiments, the event handler 200 is configured to process messages originating from or addressed to a transcription system by passing them to the transcription system interface 206. In these embodiments, the transcription system interface 206 is configured to exchange messages with a remote transcription system via an application program interface (API) exposed by the transcription system. For examples, the API may be implemented as a web services API, although other technologies may be used for this purpose. The transcription system interface 206 can thereby transmit information, such as media files storing content documenting a patient encounter, to the transcription system and/or receive information, such as transcripts of media files previously provided to the transcription system. Examples of transcription systems that the transcription system interface 206 is configured to exchange messages with include the transcription system 2200 described further below with reference to FIG. 22. In these examples, the messages transmitted via the transcription system interface 206 may include transcription request information that is processed by the transcription system 2200 as described further below.

In some embodiments, the event handler 200 is configured to process messages originating from or addressed to an ASR system or device by passing them to the ASR system interface 208. In these embodiments, the ASR system interface 208 is configured to exchange messages with a remote ASR system or local ASR device via an application program interface (API) exposed by the ASR system or device. The ASR system interface 208 can thereby transmit information, such as media files storing content documenting a patient encounter, to the ASR system or device and/or receive information, such as automatically generated transcripts, transcription confidence metrics, and the like, from the ASR system or device. Examples of ASR systems and devices that the ASR system interface 208 is configured to exchange messages with include those provided by Speechmatics, Nuance (DragonDictate), and IBM (Watson STT). Such ASR systems may provide “raw” speech-to-text capability and may also be supplemented by post-processing steps that are designed to improve the formatting and accuracy of the output text. An exemplary system possessing of this latter capability is the transcription system 2200 described further below with reference to FIG. 22, one example of which is available from 3Play Media. ASR systems may operate either in real time, streaming text back over a web socket in response to media received over that socket, or in batch mode, where the entire media file is received and processed, and the entire transcript is posted back to the mobile recording application 118 via the ASR system interface 208.

In some embodiments, the voice macro processor 210 is configured to search transcript text for trigger text and replace the trigger text with expansion text. Voice macros can be used to insert substantial blocks of text and can insert text within multiple sections of the EHR or other transcripts of divisible content. The voice macro processor 210 may implement any of a variety of search and replace processes to accomplish its function. For instance, in some embodiments, the voice macro processor 210 is configured to search transcript text only for exact matches of trigger text.

Alternatively or additionally, in some embodiments, the voice macro processor 210 is configured to identify and expand transcript text that is not an precise match of trigger text. In some of these embodiments, the voice macro processor 210 is configured to use a regular expression grammar to expand the trigger text into multiple possible valid sets of trigger text which can be identified and expanded. For example, if the trigger text is “Insert my standard review of systems”, in some embodiments, the voice macro processor 210 represents this trigger text as the regular expression:

(please)?(use|insert)((my|the))?(standard|normal)(review(of)?systems?|ROS)((template|macro))?

In this example, the voice macro processor 210 would identify transcript text such as “insert standard review system” as matching the trigger text and replace the transcript text with expansion text.

In another embodiment, when processing a voice macro (e.g., “Please insert the normal PE with BP one ten over eighty weight one hundred forty pounds five feet six”), the voice macro processor 210 is configured to, in addition to inserting the normal expansion text, replace variables in the transcript text (e.g., variables associated with “VITAL SIGNS”) using natural language processing (NLP) techniques. For instance, the voice macro processor 210 may execute keyword/sequence spotting (e.g., for “blood pressure,” “weight,” “height,” etc.) and for each keyword/sequence execute numeric parsing (e.g., for “one ten over eighty,” “one hundred forty pounds,” “five feet six”) replace variables with literals. In some embodiments, the voice macro processor 210 may infer any one or more of these attributes from the word sequence using, for example an n-gram approach.

In some embodiments, the association engine 212 implements features of the metadata association system 100 described in the Metadata Media Associator patent. More specifically, in these embodiments, the association engine 212 is executable by the event handler 200 to create links between portions of transcript text and metadata such as digital images, SNOMED codes, or other digital content. In at least one embodiment, the association engine receives, from the event handler, an identifier of a portion of transcript text and an identifier of the metadata and, in response, generates XML linking the identifiers. This XML may later be parsed by, for example, the user interface component 202 to render the metadata in conjunction with the transcript text. Thus, in this embodiment, the user interface component 202, the event handler 200, and the association engine 212 interoperate to execute a metadata association process, such as the process 500 described in the Metadata Media Associator patent.

### Flexible Recording Interface

Certain embodiments of the mobile recording application 118 implement a flexible recording interface that enables a health care provider to efficiently record audio entries for the EHR record at a variety of times and locations. The flexible recording interface solves several technical challenges faced by conventional transcription system interfaces. For instance, transcriptions of noisy recordings generated by conventional ASR typically have an unacceptable number of errors. By allowing a user (e.g., the user 126) to select a level of review to be performed on particular sections of audio entries, the flexible recording interface overcomes this technical issue by engaging human editors to remove errors only when needed. Additionally, conventional transcription system interfaces are designed to allow user to record a wide variety of content. While such designs support a wide variety of uses, they also inhibit productivity for specialized users. By presenting a design tailored to the recording of audio entries for specific sections of the EHR, at least some embodiments of the flexible recording interface overcome technical inefficiencies endemic in conventional transcription system interfaces. Moreover, some embodiments segment audio entries into distinct sections which may be identified by one or more tags. This segmentation can help solve challenges related to completing quality transcriptions at scale. For example, where audio entries may be grouped into distinct sections, at scale, specialists for each section can focus their review only on audio entries belonging to their section of specialty. Or if particular sections use a tag like “confidential” or “restricted” instead of “HPI” or “Assessment and Plan”, different security classifications could be applied for content access based on the tags.

FIG. 3 illustrates a home screen 300 presented by at least one embodiment of the flexible recording interface as implemented by at least one example of a mobile recording application (e.g., the mobile recording application 118). As shown in FIG. 3, the home screen 300 is sized and arranged for a display (e.g., the display 110) of a mobile computing device (e.g., the mobile computing device 100). In some embodiments, prior to presenting the home screen 300, the event handler interoperates with a remote EHR system (via the transcript database system interface 204) to retrieve schedule data for the user. This schedule data may be stored in the schedule data store 120 and may include appointment data that represents patient appointments scheduled for the user. This appointment data may include data representative of appointment times, patient names, and/or patient check-in status.

The home screen 300 is segmented into an app header 302, a screen header 304, and a body 306. The app header 302 includes a calendar control 308 and a settings control 310. The screen header 304 includes a title of the screen, “Home.” The body 306 includes a daily appointments control 312, a search dictations control 314, and a manage settings control 316.

In some examples, an event handler (e.g., the event handler 200) of the mobile recording application is configured to present the home screen 300 by interoperating with a user interface component (e.g., the user interface component 202) of the mobile recording application. For instance, the event handler may present the home screen 300 upon boot of the mobile recording application and at various other times depending on the interaction between the user and the mobile computing device.

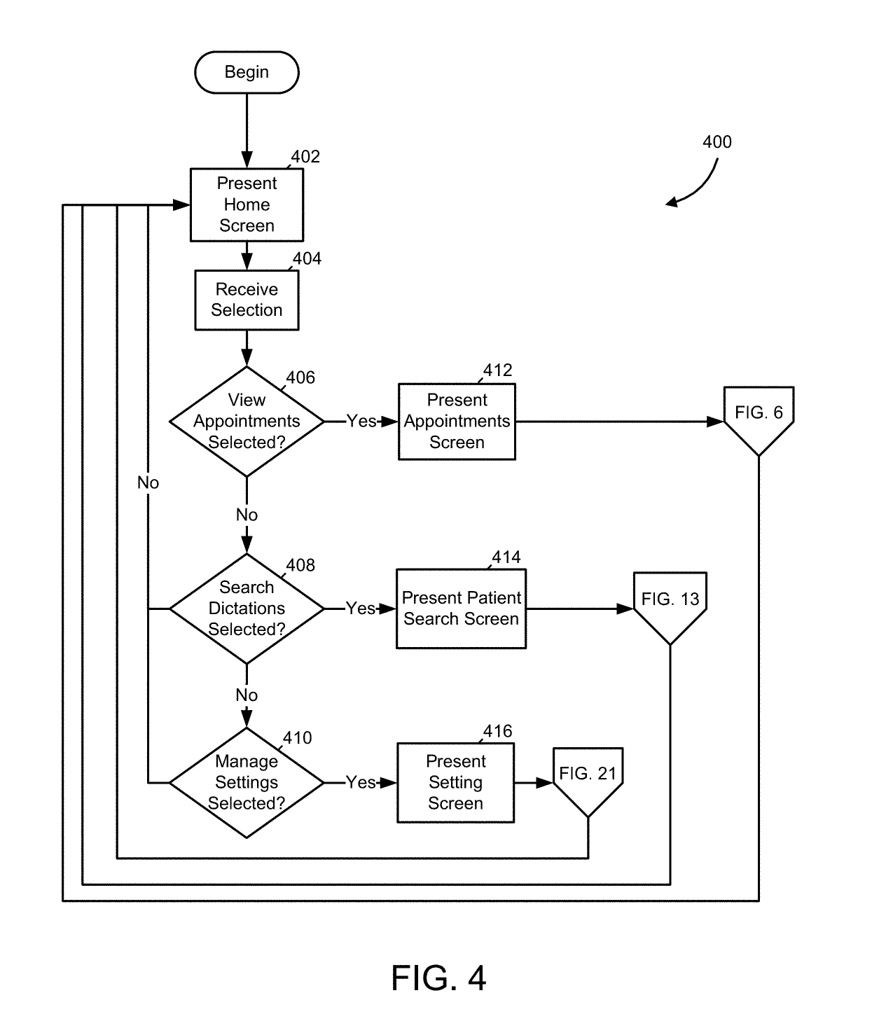

When called upon to present the home screen 300 (e.g., when the mobile recording application initially boots), the event handler interoperates with the user interface component to execute an interface process 400 that is illustrated in FIG. 4. As shown in FIG. 4, the interface process 400 starts in act 402 with the event handler presenting the home screen 300 via the user interface component and display.

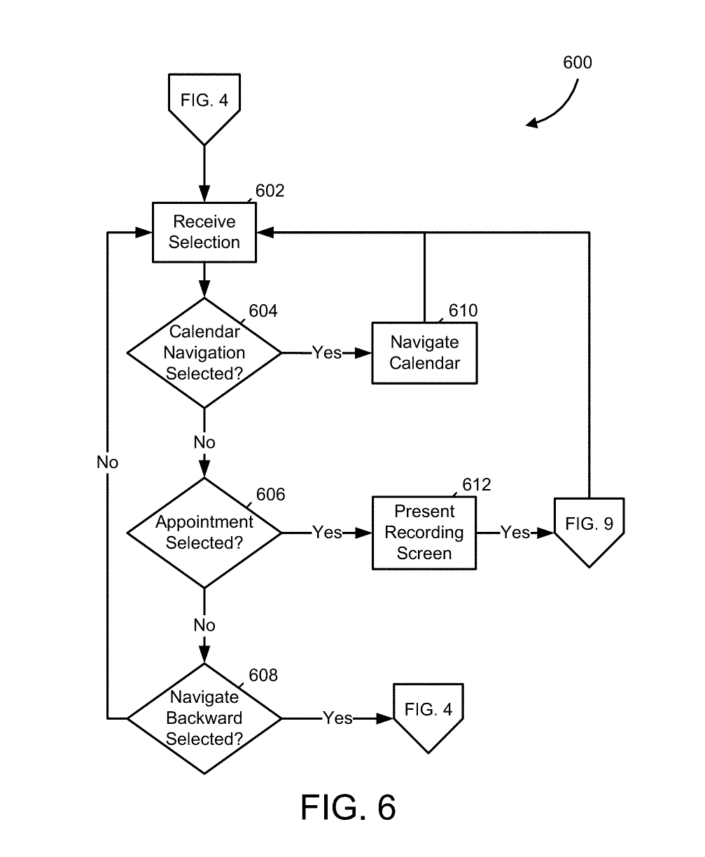

In act 404, the event handler receives (e.g., via the display and the user interface component) a selection of an element of the home screen 300 (e.g., indicated by user input). In act 406, the event handler determines whether daily appointments control 312 was selected. If so, in act 412 the event handler presents an appointments screen (e.g., the appointments screen 500 described further below) and proceeds to an interface process 600 described below with reference to FIG. 6. Otherwise, in act 408 the event handler determines whether the search dictations control 314 was selected.

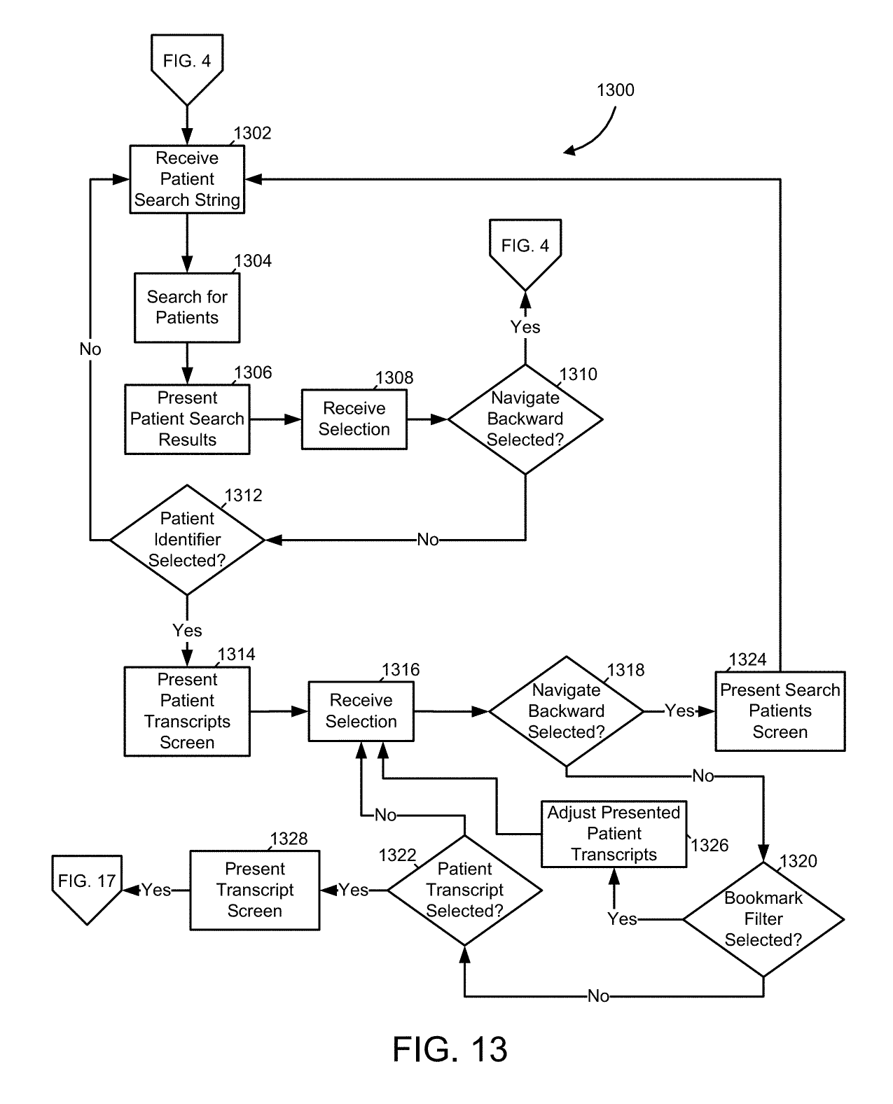

If the event handler determines that the search dictations control 314 was selected, the event handler, in act 414, presents a patient search screen (e.g., the patient search screen 1200 described further below) and proceeds to an interface process 1300 described below with reference to FIG. 13. Otherwise, in act 410 the event handler determines whether the manage settings control 316 was selected.

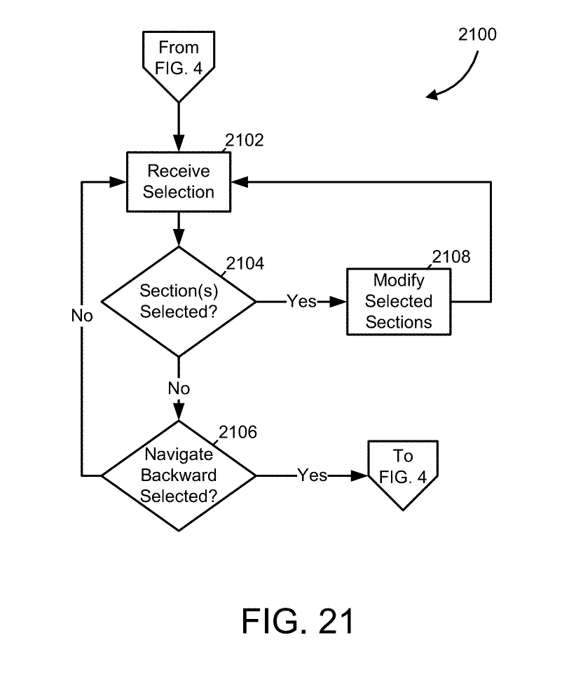

If the event handler determines that the manage settings control 316 was selected, the event handler, in act 416, presents a settings screen (e.g., the transcript defaults screen 2000 described further below) and proceeds to an interface process 2100 described below with reference to FIG. 21. Otherwise, the event handler returns to the act 402, and the interface process 400 reiterates.

In some embodiments, the event handler is further configured to process selections of the calendar control 308 and the setting control 310. When executing according to this configuration, if the event handler determines that the calendar control 308 was selected, the event handler displays a calendar to enable a user to select a date other than the current date for which the daily appointments screen will be presented. If the event handler determines that the setting control 310 was selected, the event handler presents the setting screen. The event handler may be configured to process selections of the calendar control 308 and the setting control 310 in the manner recited above when presenting other screens (e.g., 500, 700, 800, 1000, 1200, 1400, 1500, 1600, 1700, 1900) described herein.

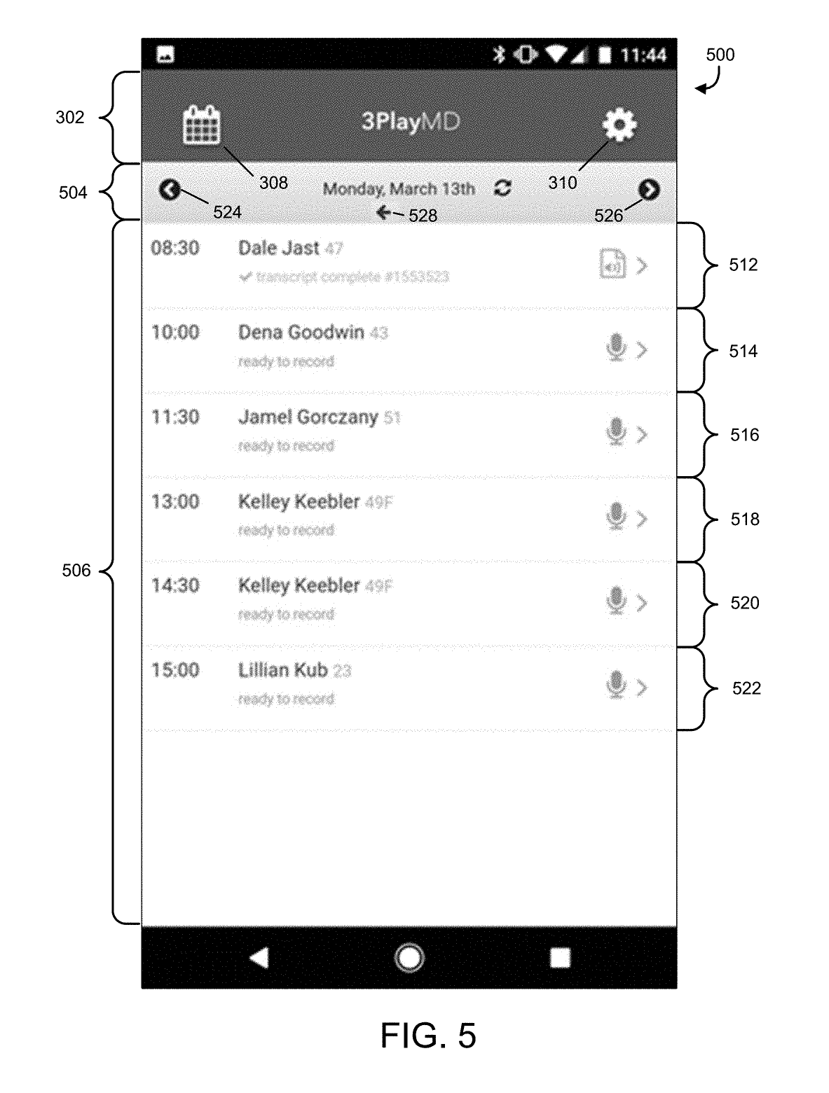

FIG. 5 illustrates the appointments screen 500 as presented by at least one embodiment of the flexible recording interface. The appointment screen 500 includes some elements similar to the elements of the home screen 300 (e.g., the app header 302, the calendar control 308, and the settings control 310). These elements of the appointment screen 500 are structured and function like the elements of the home screen 300.

The appointments screen 500 is segmented into the app header 302, a screen header 504, and a body 506. The screen header 504 includes an indicator of the date for which appointment information is displayed in the body 506, two calendar navigation controls 524 and 526, and a backward navigation control 528. The body 506 includes a series of appointment controls 512-522 displaying the appointment information. Each appointment control of the series of appointment controls 512-522 represents an appointment scheduled for the user. Each appointment may correspond to a scheduled patient encounter. As shown in FIG. 5, each appointment control includes an indicator of a start time for an appointment, an identifier of a patient to be encountered during the appointment, an indicator of the status of the EHR record for the patient encounter (e.g., “transcript complete,” “transcript in process,” “transcript pending,” or the like) and an indicator of the check-in status of the patient (e.g., “ready to record”).

As shown in FIG. 5, the appointments screen 500 is sized and arranged for the display of the mobile computing device. For example, the appointment controls 512-522, which are described further below, are designed for full screen width. This enables the user to easily operate the appointments screen 500 using one hand. For instance, the user can use his or her thumb to scroll, swipe, and navigate the calendar, all while the user moves from location to location.

When presenting the appointments screen 500, the event handler interoperates with the user interface component to execute an interface process 600 that is illustrated in FIG. 6. As shown in FIG. 6, the interface process 600 starts in act 602 with the event handler receiving a selection of an element of the appointments screen 500.

In act 604, the event handler determines whether either of the calendar navigation controls 524 or 526 was selected. If the calendar navigation control 524 was selected, in act 610 the event handler presents the appointments screen 500 for the day prior to the date indicated in the screen header 504. If the calendar navigation control 526 was selected, in act 610 the event handler presents the appointments screen 500 for the day after the date indicated in the screen header 504. If neither of the calendar navigation controls 524 and 526 was selected, in act 606 the event handler determines whether an appointment control of the series of appointment controls 512-522 was selected.

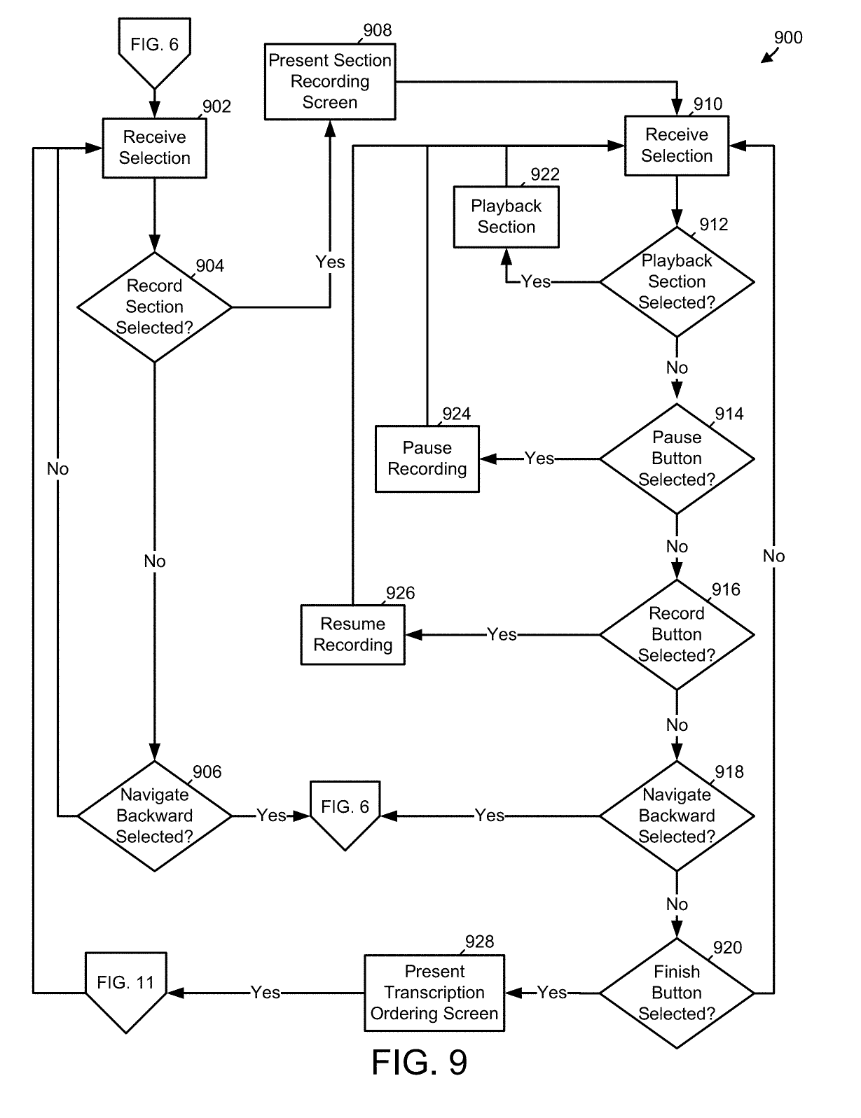

If the event handler determines that an appointment control of the series of appointment controls 512-522 was selected, the event handler, in act 612, presents a recording screen (e.g., the recording screen 700 described further below) and proceeds to an interface process 900 described below with reference to FIG. 9. Otherwise, in act 608 the event handler determines whether the backward navigation control 528 was selected. If the event handler determines that the backward navigation control 528 was selected, the event handler returns to the interface process 400 described above with reference to FIG. 4. Otherwise, the event handler returns to the act 602, and the interface process 600 reiterates.

In some embodiments, when presenting the appointments screen 500, the event handler continuously updates indicators of patient/transcript status. In these embodiments, the event handler receives streamed data via an ASR system interface (e.g., the ASR system interface 208) and/or a transcript database system interface (e.g., the transcript database system interface 204) and updates the elements of the appointments screen based on the streamed data. Thus, in these embodiments, using the appointments screen 500 a user can identify the percentage of completeness of transcript of a patient encounter in near real-time.

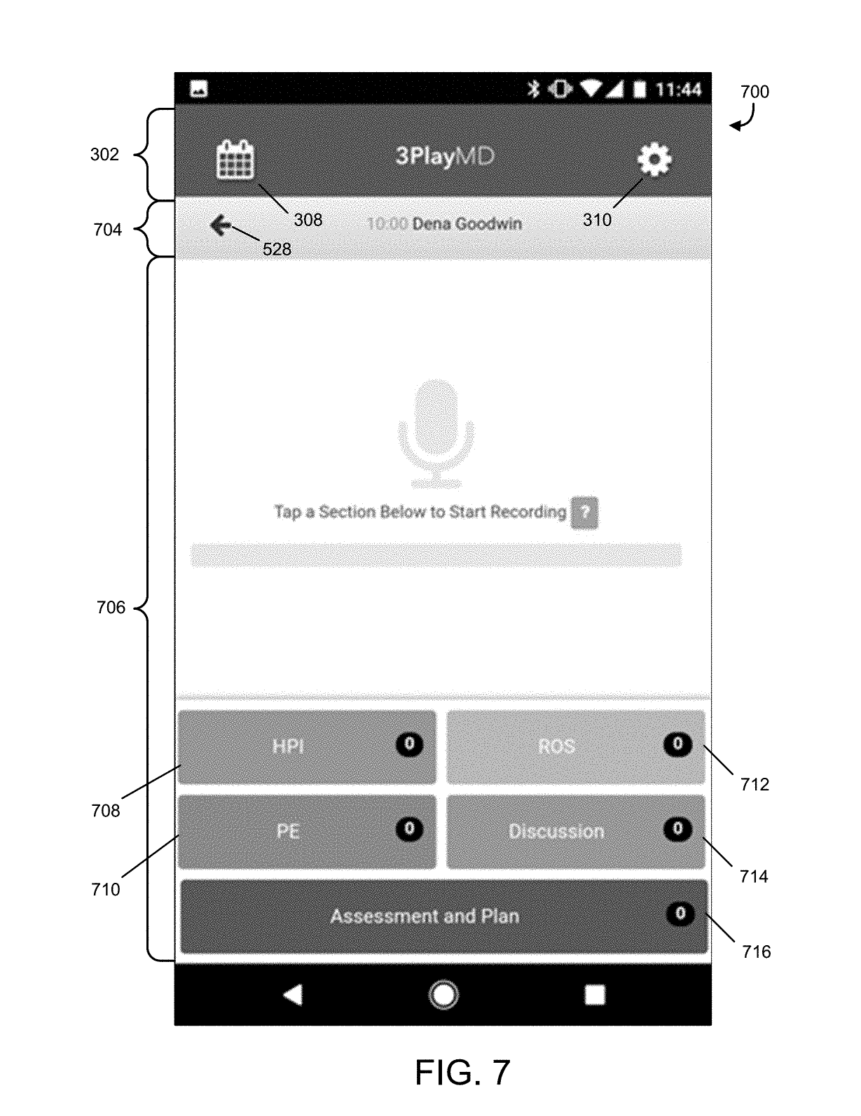

FIG. 7 illustrates the recording screen 700 as presented by at least one embodiment of the flexible recording interface. The recording screen 700 includes some elements similar to the elements of the appointments screen 500 (e.g., the app header 302, the calendar control 308, the settings control 310, and the backward navigation control 528). These elements of the recording screen 700 are structured and function like the elements of the appointments screen 500.

The recording screen 700 is segmented into the app header 302, a screen header 704, and a body 706. The screen header 704 includes an indicator of the patient who is the subject of the audio entries to be recorded via the recording screen 700 and an indicator of the time of the patient encounter. The body 506 includes section recording controls 708-716. Each of the recording controls 708-716 represents a distinct section of the EHR documenting this patient encounter. As shown in FIG. 5, each recording controls 708-716 includes a section identifier and an indicator of the recording status of the EHR for the section identified. More specifically, the recording control 708 represents the History of Present Illness (HPI) section. The recording control 710 represents the Physical Examination (PE) section. The recording control 712 represents the Review of Systems (ROS) section. The recording control 714 represents the Discussion section. The recording control 716 represents the Assessment and Plan section. Each of the recording controls 708-716 indicate that no audio entries have been recorded for that section by including an indicator of “0.”

As shown in FIG. 7, the recording screen 700 is sized and arranged for the display of the mobile computing device. For example, the positioning of the recording controls 708-716 at the bottom of the screen is designed for a user who is moving from location to location (e.g., in a doctor's office). Such a user may only be able to operate the device with one hand (e.g., a user holding the phone in his or her right hand and operating the phone exclusively with the thumb). For this reason, the recording controls 708-716 are rendered in a larger design and consume a minimum screen width of 50% for each.

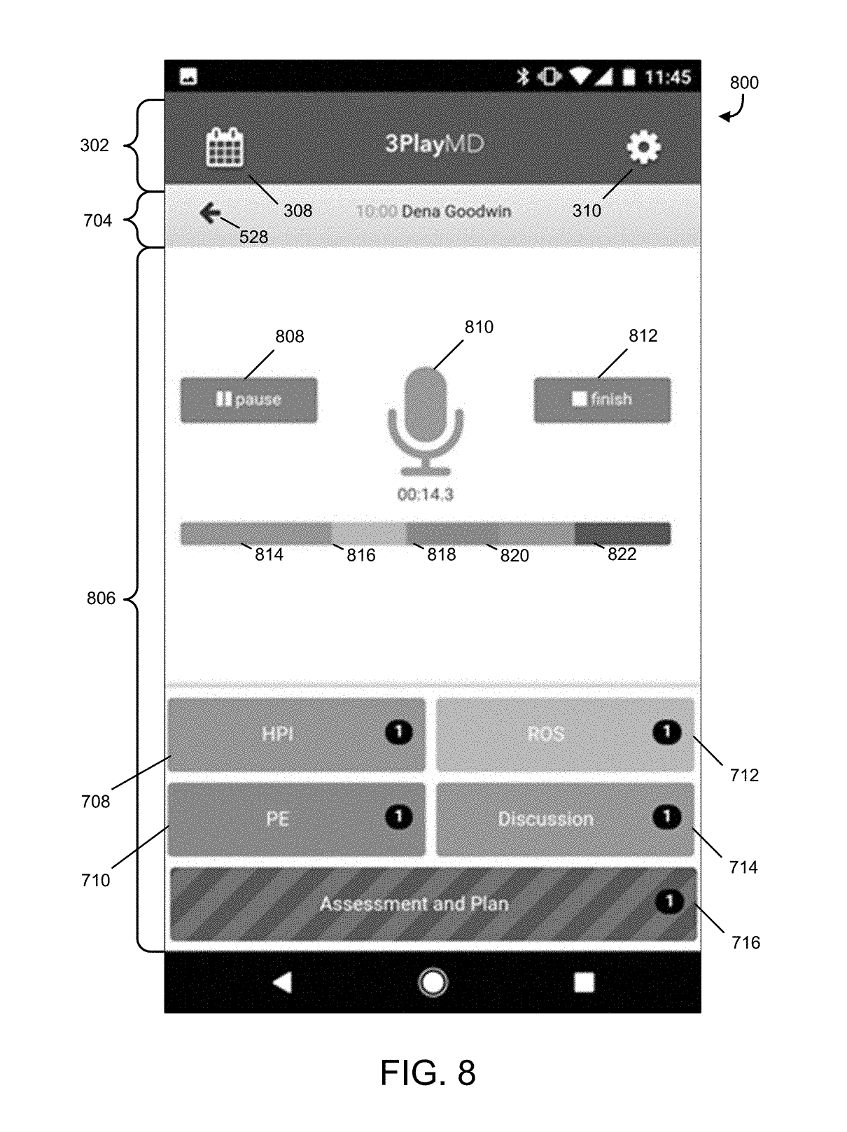

FIG. 8 illustrates another recording screen 800 as presented by at least one embodiment of the flexible recording interface. The recording screen 800 includes some elements similar to the elements of the recording screen 700 (e.g., the app header 302, the calendar control 308, the settings control 310, the backward navigation control 528, the screen header 704, and the section recording controls 708-716). These elements of the recording screen 800 are structured and function like the elements of the recording screen 700.

The recording screen 800 is segmented into the app header 302, the screen header 704, and a body 806. The body 806 includes a pause control 808, a record control 810, a finish control 812, playback section controls 814-822, and the section recording controls 708-716. As shown in FIG. 8, the recording control 716 is highlighted (via diagonal stripes) to indicate that the assessment and plan section is currently being recorded. In some embodiments, the pause control 808, the record control 810, and/or the finish control 812 may be shaded the same color as the section being currently recorded. Each of the recording controls 708-716 indicate that at least one audio entry has been recorded for that section by including an indicator of “1.” Also as shown in FIG. 8, the playback section controls 814-822 are rendered in colors corresponding to the colors of the section recording controls 708-716.

As shown in FIG. 8, the recording screen 800 is sized and arranged for the display of the mobile computing device. Since the hand of the user may block part of the recording screen 800 (e.g., the section recording controls 708-716), the recording feedback elements (e.g., the pause control 808, the record control 810, the finish control 812, and the playback section controls 814-822) is above where the hand position tends to be, so the user is able to affirm a color change when a new section recording control is tapped. The app header 302, the calendar control 308, the settings control 310, and the backward navigation control 528 are positioned out of the way as these controls are less frequently used and meant more for a user who has time to change operational modes.

During the presentation of the recording screens 700 and 800, the event handler interoperates with the user interface component to execute an interface process 900 that is illustrated in FIG. 9. As shown in FIG. 9, the interface process 900 starts in act 902 with the event handler receiving a selection of an element of the recording screen 700.

In act 904, the event handler determines whether one of the section recording controls 708-716 was selected. If one of the section recording controls 708-716 was selected, in act 908 the event handler presents the recording screen 800 with the selected section recording control highlighted, stores a timestamp to mark the beginning of the section recording, and starts recording (e.g., via the user interface component and the microphone 112) an audio entry for the EHR section represented by the selected section recording control. In some embodiments, the event handler also streams (e.g., via the ASR System Interface 208) the audio entry to an ASR system or device. In these embodiments, the event handler receives transcript text in near real-time for subsequent processing. Alternatively or additionally, the event handler may record freeform text from a keyboard. If none of the section recording controls 708-716 was selected, in act 906 the event handler determines whether the backward navigation control 528 was selected. It is appreciated that the user can randomly transition between EHR sections to record audio entries for each section in any order by simply selecting the desired section recording control. Storing timestamps at the beginning of each section transition enables distinct, non-sequential entries into the various sections to be properly organized into appropriate EHR sections, as described further below.

If the event handler determines that the backward navigation control 528 was selected, the event handler returns to the interface process 600 described above with reference to FIG. 6. Otherwise, the event handler returns to the act 902, and the interface process 900 reiterates.

In act 910, the event handler receives a selection of an element of the recording screen 800. In act 912, the event handler determines whether one of the playback section controls 814-822 was selected. If one of the playback section controls 814-822 was selected, in act 922 the event handler renders (e.g., via the user interface component and the speaker 116) the audio entries for the EHR section represented by to the selected playback section control. More specifically, if the playback section control 814 was selected, the event handler renders the audio entries for the HPI section. If the playback section control 816 was selected, the event handler renders the audio entries for the ROS section. If the playback section control 818 was selected, the event handler renders the audio entries for the PE section. If the playback section control 820 was selected, the event handler renders the audio entries for the Discussion section. If the playback section control 822 was selected, the event handler renders the audio entries for the Assessment and Plan section.

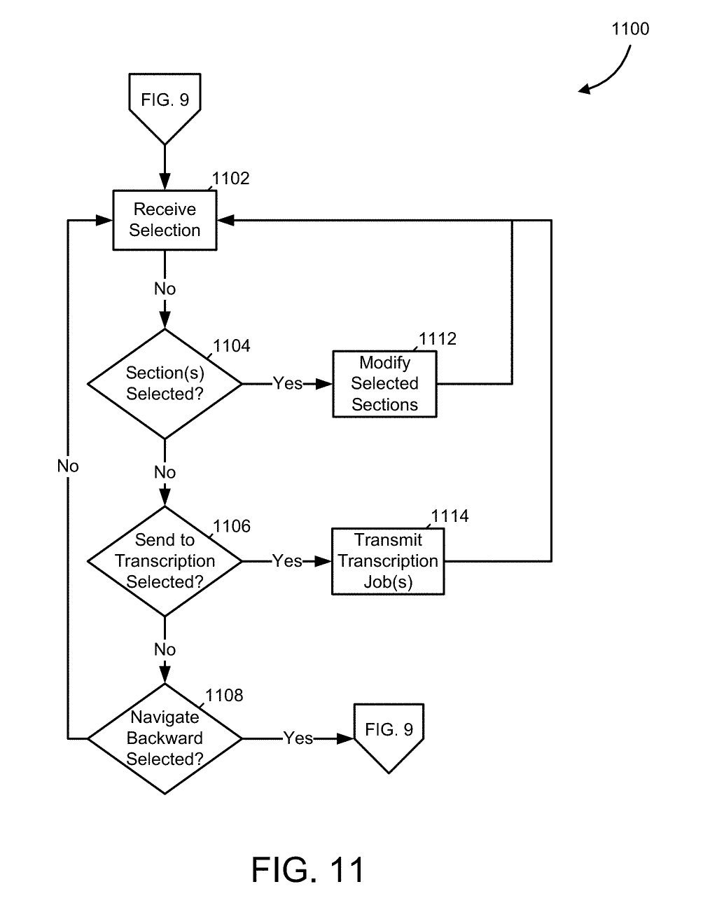

If none of the playback section controls 814-822 was selected, in act 914, the event handler determines whether the pause control 808 was selected. If so, in act 924 the event handler pauses recording the audio entry for the EHR section and returns to the act 910. Otherwise, in act 916 the event handler determines whether the record control 810 was selected. If so, in act 926 the event handler resumes recording of the audio entry for the EHR section. Otherwise, in act 918 the event handler determines whether the backward navigation control 528 was selected. If the event handler determines that the backward navigation control 528 was selected, the event handler returns to the interface process 600 described above with reference to FIG. 6. Otherwise, in act 920 the event handler determines whether the finish control 812 was selected. If so, in act 928 the event handler presents a transcription ordering screen (e.g., the transcription ordering screen 1000 described further below) and proceeds to an interface process 1100 described below with reference to FIG. 11. Otherwise, the event handler returns to the act 910.

In some embodiments, when presenting the recording screens 700 and 800, the event handler is configured to process audio entries in real time and identify (e.g. using natural language processing techniques) words and phrases that indicate section transitions. Where the event handler identifies a section transition in this manner, the event handler stores a timestamp to mark the transition. Words and phrases that the event handler is configured to use to identify section transitions may include words and phrases descriptive of the sections themselves or words and phrases articulating content normally found within particular sections. In some examples, these section words and phrases are configurable. Examples of section words and phrases include “Review of Systems” and “Now for ROS” for a transition to the ROS section. Another example section phrase includes “Vital signs. Pulse 72, BP 120/80” for a transition to the PE section.

In some embodiments, the event handler is configured to search for section words and phrases as regular expressions. For instance, particular values of the pulse and blood pressure may be treated as a regular expression, for example /\d+/ for the pulse or /\d+\/\d+/ for the blood pressure. In this example, the event handler would identify “Pulse 72, BP 120/80” using this regular expression. Additionally, valid ranges for those values may be used to further identify valid transitional phrases.

In some embodiments, the event handler is configured to identify section words and phrases using probabilistic techniques, with each phrase indicating some likelihood for all possible section transitions. In these embodiments, the event handler may also incorporate section-sequencing probabilities, for example by using an N-gram formulation to indicate the relative probabilities of sections occurring in a given order. Combinations of these and other constraints (such as section duration modeling) may be implemented using statistical formulations such as Bayes' rule or by search algorithms such as the Viterbi algorithm. It is appreciated that the techniques described above may be implemented using either real-time ASR or batch ASR (with or without additional human editing), as described in the Electronic Job Market application.

In another embodiment, the event handler is configured to use manual and automatic processes to identify EHR section transitions. For instance, the event handler may be configured to receive a selection of the recording control 708 and create a timestamp marking a transition to the HPI section. The event handler may continue to record while receiving audio entries for other EHR sections until receiving a selection of the recording control 716 indicating a transition to the Discussion section, responsively create a timestamp marking the transition, and continue to record while receiving audio entries for the Assessment and Plan section without receiving a selection of the recording control 716. In this example, the event handler is configured to automatically identify the transitions to the PE, ROS, and Assessment and Plan sections, using the reporting control selections and timestamps for the HPI and Discussion transitions as a priori anchors for the automatic processes.

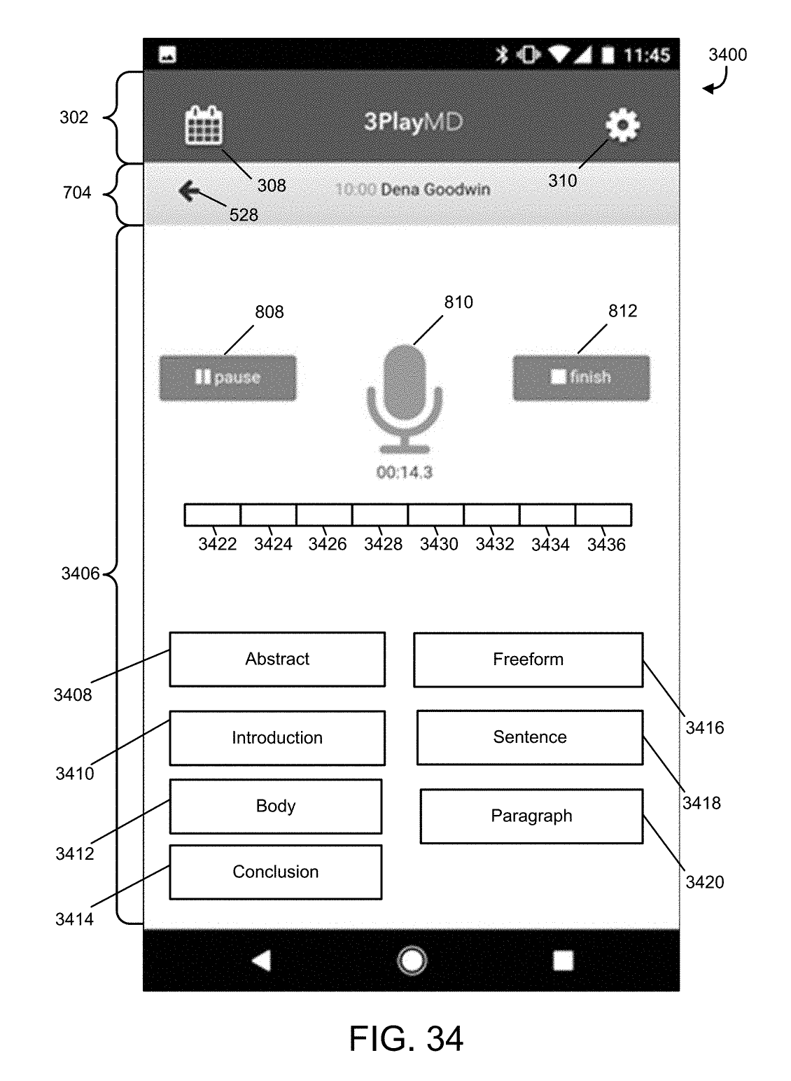

In other embodiments, the event handler is configured to use manual and automatic processes to identify other sections of a recording. FIG. 34 illustrates another recording screen 3400 as presented by these embodiments. As shown in FIG. 34, the recording screen 3400 is sized and arranged for the display of the mobile computing device. The recording screen 3400 includes some elements similar to the elements of the recording screen 800 (e.g., the app header 302, the calendar control 308, the settings control 310, the backward navigation control 528, the screen header 704, the pause control 808, the record control 810, and the finish control 812). These elements of the recording screen 3400 are structured and function like the elements of the recording screen 800.

The recording screen 3400 is segmented into the app header 302, the screen header 704, and a body 3406. The body 3406 includes the pause control 808, the record control 810, the finish control 812, playback section controls 3422-3436, and section recording controls 3408-3420. If one of the playback section controls 3422-3436 is selected, the event handler renders (e.g., via the user interface component and the speaker 116) audio entries for a section represented by to the selected playback section control. The playback section control 3422 represents the most recently recorded section and each of the remainder of the playback section controls 3424-3436 represents a section recorded adjacent and prior to the section represented by the playback section control to its left.

If one of the section recording controls 3408-3420 is selected, the event handler highlights the selected section recording control, stores a timestamp to mark the beginning of the section recording, and starts recording (e.g., via the user interface component and the microphone 112) an audio entry for the section represented by the selected section recording control. In some embodiments, the event handler also streams (e.g., via the ASR System Interface 208) the audio entry to an ASR system or device. In these embodiments, the event handler receives transcript text in near real-time for subsequent processing.

For instance, in response to receiving a selection of the section recording control 3420, the event handler creates a timestamp marking a transition to a new paragraph of the recording (and subsequently generated transcript). The event handler may continue to record the next paragraph until receiving a selection of the section recording control 3420 indicating a transition to another paragraph, responsively create a timestamp marking the transition, and continue to record while receiving audio entries for this new paragraph. Or, the event handler may receive a selection of the section recording control 3414 to create a timestamp marking a transition to a Conclusion section. Similarly, the event handler may receive a selection of the section recording control 3418 to create a timestamp marking a sentence boundary. The event handler may continue to record the next sentence until receiving a selection of the section recording control 3418 indicating a transition to another sentence, responsively create a timestamp marking the transition, and continue to record while receiving audio entries for this new sentence.

These transitions to new sentences, paragraphs or other labelled sections (e.g., Abstract, Introduction, Body, Freeform, etc.) of a recording and eventual transcript may also be determined by the event handler automatically using approaches such as punctuation modeling, topic identification or keyword matching, based on the streaming output of the ASR system or device, in combination with natural language processing models. For example, a topic model may be used to determine that the words spoken by the user have transitioned to a new topic, and this determination may then be used to communicate with the event handler to transition to the next paragraph. Or the ASR system may be configured to identify sentence boundaries using, e.g. language modeling, prosodic modeling, and/or parsing techniques. Or, the ASR system may be configured to trigger communication to the event handler based on a keyword phrase (expressed as a regular expression), such as:

<B>((IN)?(CONCLUSION|SUMMARY))|(TO(CONCLUDE|SUMMARIZE))

where the <B> symbol indicates an automatically detected sentence boundary. In this example, detection of words matching this regular expression would cause the ASR system to communicate with the event handler to transition to the Conclusion section of the transcript document. If using a non-real-time ASR system, these transitions can be performed in batch mode by the ASR and NLP components, segmenting the document appropriately for later display to the user.

Several advantages may ensue from sectioning the transcript in this way. For example, based on the sectioning (either manual or automatic), distinct language models can be applied during ASR processing that take into account the specific domain of language in the section. For example, in a “Review of Systems” section, the ASR system or device could apply a language model (either initially, or as a postprocessor on a lattice produced using a more general language model) that accounts for the terms typically used in that section. Additionally, a formatting postprocessor could be selected which is optimized for a given section. For example, a “Physical Examination” formatting postprocessor could be applied which would include knowledge of the formats required for such quantities as blood pressure, temperature, height and weight. Similarly, in the case where a topic model is used to automatically identify a new paragraph in a transcript, a topic-tuned language model could be applied by the ASR system or device to improve accuracy.

In some embodiments, the event handler uses audio entry JSON objects to manipulate transcript text. For instance, when presenting recording screens 700 and 800, for recording controls that are selected (or, as described above, in some embodiments, sections that are automatically identified), the event handler is configured to create an audio entry JSON object indicating the current time in the recording as well as the section selected. An array of these audio entry JSON objects is collected until the recording is finished, e.g. [{“time_milliseconds”: 0, “section”: “HPI”}, {“time_milliseconds”: 32500, “section”: “PE”}, {“time_milliseconds”: 65000, “section”: “ROS”}, {“time_milliseconds”: 102400, “section”: “Discussion”}, {“time_milliseconds”: 145670, “section”: “ROS”, “continuation”: true}, {“time_milliseconds”: 190450, “section”: “Assessment and Plan”}]. In this example, we see that the ROS section is represented in two elements of the JSON array, with the second element indicating a continuation of the section. The event handler may construct the final transcript for the patient encounter by moving the text corresponding to the 44780 milliseconds of the continuation section to immediately succeed the initial 37400 milliseconds. This text rearrangement may also be used at transcript editing time (where human editing is selected for the ROS section), as this may be advantageous for the editor in understanding the full context of the section. In this example, the event handler may also rearrange the audio entries to correspond to the transcript during playback of the ROS section (e.g., in response to selection of the playback section control 814 described below with reference to FIG. 8), so that the user can hear the entire ROS section continuously. Alternatively or additionally, the JSON document may represent numbered paragraphs and/or sections, e.g. [{“time_milliseconds”: 123589, “section”: “paragraph_1”},{“time_milliseconds”: 389568, “section”: “paragraph_2”},{“time_milliseconds”:983456, “section”: “Conclusion”}].

Once audio entries documenting a patient encounter or other transcript information are complete (e.g., the event handler receives a selection of the finish control 812), the user can select which sections to transcribe only by a machine and which sections to transcribe by machine and human review. Machine only transcriptions are less expensive but often contain some level of error. Human review transcriptions are more expensive but very accurate. As is described in more detail below, in some embodiments, the event handler is configured to transmit media files containing the audio entries to the 3Play Media transcription system (e.g., the transcription system 2200 described further below). These media files may include distinct media files per EHR section or combined media file including two or more EHR sections along with section timestamp information. The transcription system extracts relevant portions of the audio, generates an ASR draft transcript for each section, stores the ASR draft transcripts as final transcripts for sections selected as machine only, and submits editing jobs for sections selected for human review. In these embodiments, when the human transcription is completed, the final, full transcript is created by concatenating the human and automated transcripts together, by EHR section as defined by the transition timestamps. The sections present within the final, full transcript may be transmitted to an external, remote EHR system through an EHR system interface, such as the transcript database system interface 204 or the transcript database system interface 2240 described further below with reference to FIG. 22.

In another embodiment, the sections of the transcript may be sentences, paragraphs or other titled sections (e.g. Introduction, Section 3, Conclusion, etc.) and the final transcript, which combines fully-automated and human-corrected sections, may be stored in a database.

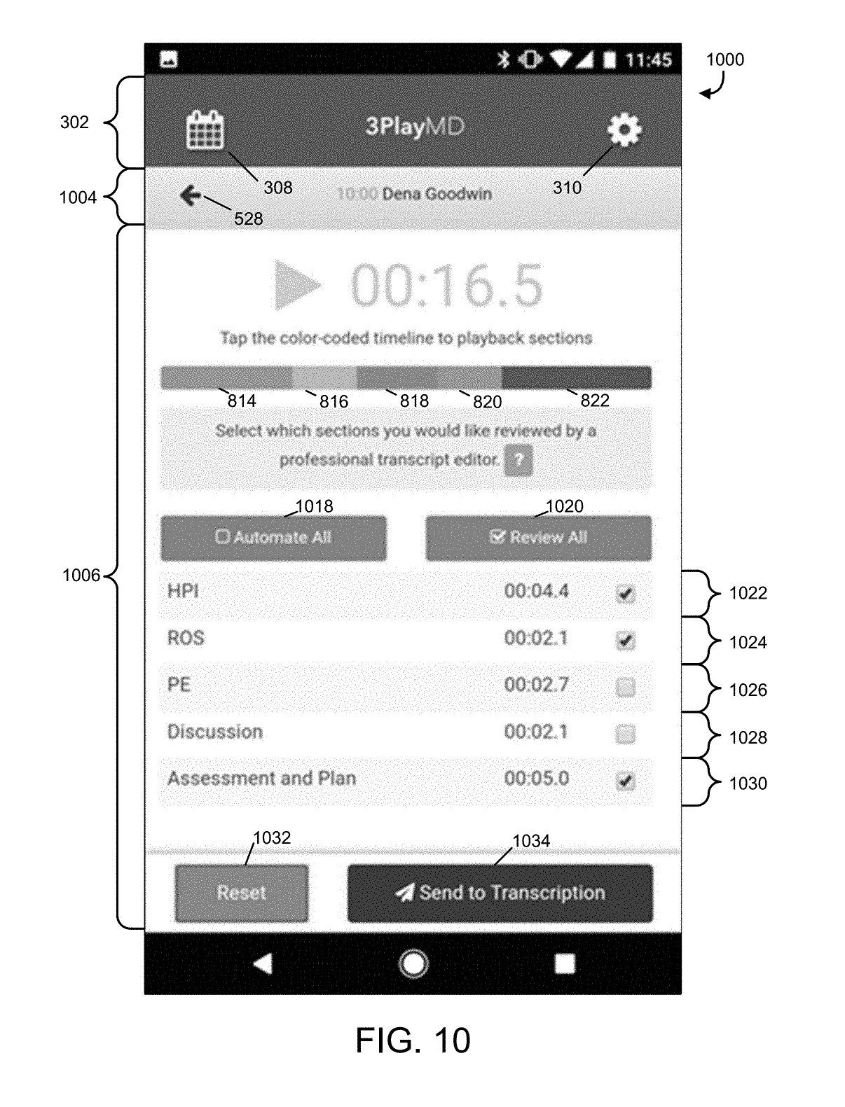

FIG. 10 illustrates the transcription ordering screen 1000 as presented by at least one embodiment of the flexible recording interface. The transcription ordering screen 1000 includes some elements similar to the elements of the recording screen 800 (e.g., the app header 302, the calendar control 308, the settings control 310, the backward navigation control 528, and the playback section controls 814-822). These elements of the transcription ordering screen 1000 are structured and function like the elements of the recording screen 800.

The transcription ordering screen 1000 is segmented into the app header 302, a screen header 1004, and a body 1006. The screen header 1004 includes the backward navigation control 528 and an indicator of the patient appoint documented by the audio entries into the EHR record listed in the body 1006. The body 1006 includes playback section controls 814-822, section selection controls 1018-1032, and order transcription control 1034. As shown in FIG. 10, the section selection controls 1020, 1022, 1024, and 1030 are selected, as indicated by the checkmark displayed in each.

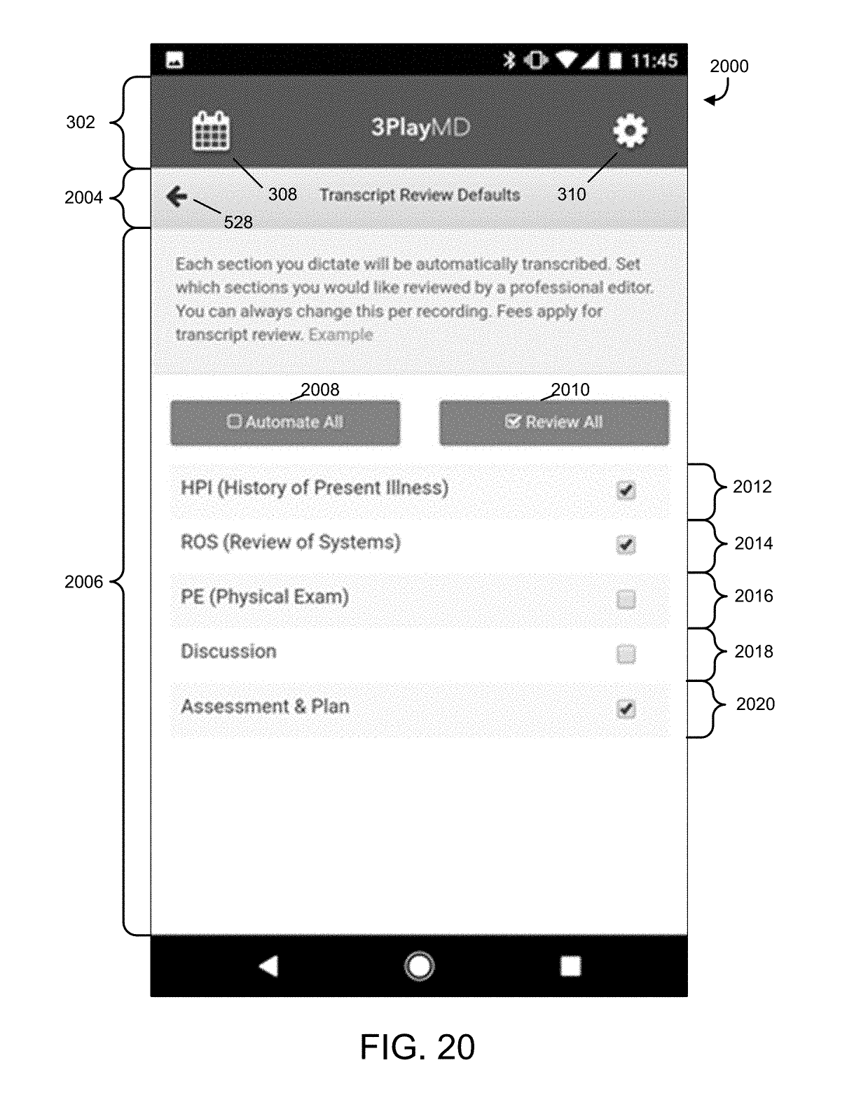

As shown in FIG. 10, the transcription ordering screen 1000 is sized and arranged for the display of the mobile computing device. As with the recording screens 700 and 800, the overall layout of the transcription ordering screen 100 is designed to be easily operated by a user using one hand. As shown, the order transcription control 1034, which is the most frequently tapped control on the transcription ordering screen 1000, is rendered in a large design for ease of use. As is described further below, tapping the order transcription control 1034 orders a transcript with a set of default sections requested for review. The presence of the default sections, which are configurable via the transcript defaults screen 2000 described further below with reference to FIG. 20, enables the user to primarily use the order transcription control 1034 in a “one click” manner (i.e., without the need to tap another control). The other controls are sized according to the frequency of their use, with wide width controls being frequently used to easy one-handed operation.

During the presentation of the transcription ordering screen 1000, the event handler interoperates with the user interface component to execute an interface process 1100 that is illustrated in FIG. 11. As shown in FIG. 11, the interface process 1100 starts in act 1102 with the event handler receiving a selection of an element of the transcription ordering screen 1000.

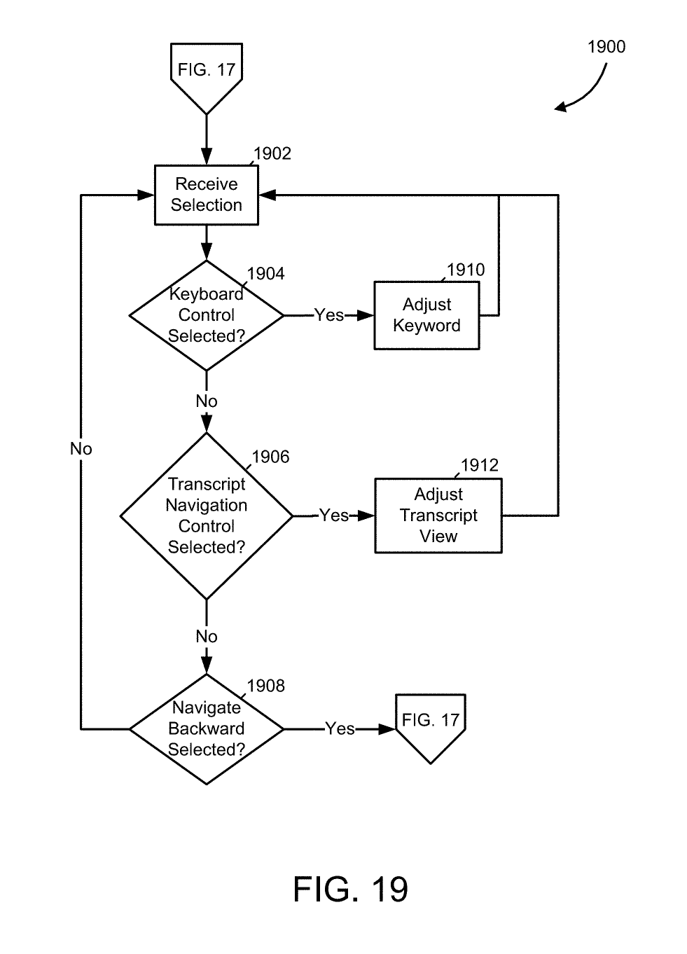

In act 1104 the event handler determines whether one of the section selection controls 1018-1032 was selected. If the event handler determines that one of the section selection controls 1018-1032 was selected, in act 1112 the event handler modifies the set of audio entries targeted for human review. More specifically, if the section selection control 1018 was selected, the event handler excludes all of the audio entries listed in the body 1006 from the set of audio entries for human review. If the section selection control 1020 was selected, the event handler includes all of the audio entries listed in the body 1006 in the set of audio entries for human review. If the section selection control 1022 was selected, the event handler toggles (e.g., excludes if currently included or includes if currently excluded) the audio entries for the HPI section relative to the set of audio entries for human review. If the section selection control 1024 was selected, the event handler toggles the audio entries for the ROS section relative to the set of audio entries for human review. If the section selection control 1026 was selected, the event handler toggles the audio entries for the PE section relative to the set of audio entries for human review. If the section selection control 1028 was selected, the event handler toggles the audio entries for the Discussion section relative to the set of audio entries for human review. If the section selection control 1030 was selected, the event handler toggles the audio entries for the Assessment and Plan section relative to the set of audio entries for human review. If the section selection control 1032 was selected, the event handler includes audio entries for a default set of EHR sections in the set of audio entries for human review. This default set of EHR sections is discussed further below with reference to FIG. 19.

In some embodiments, the effect of the specific section selection controls (i.e., section selection controls 1022-1030) overrides the effect of the broader section selection controls (i.e., section selection controls 1018, 1020, and 1032). In these embodiments, where a broader section selection control is selected, the specific section selection controls indicate the inclusion or exclusion effects of the broader section selection controls. However, the specific section selection controls can be subsequently selected to override the effect of the broader selection control. FIG. 10 illustrates one example of this feature. As shown in FIG. 10, the section selection control 1020 was initially selected to include audio entries for all of the EHR sections, but section selection controls 1026 and 1028 were subsequently selected to toggle (here, to exclude) audio entries for the PE and Discussion sections from the set of audio entries for human review.

If none of the section selection controls 1018-1032 was selected, in act 1106 the event handler determines whether the order transcription control 1034 was selected. If the order transcription control 1034 was selected, in act 1114 the event handler transmits one or more media files, via a transcription system interface (e.g. the transcription system interface 206) and/or an ASR system interface (e.g., the ASR system interface 208) for processing. More specifically, in some examples of the act 1114, the event handler transmits a single media file including all of the audio entries to a remote transcription system. In these examples, the event handler requests (e.g., via the transcription system interface) that the remote transcription system generate ASR transcripts for all audio entries. Further, in these examples, the event handler requests that the remote transcription system provide the audio entries belonging to the set of targeted audio entries to a human for review and correction. The media file and the requests described above may be transferred to the remote transcription system as transcription request information that includes one or more audio entry and/or section JSON objects as described above. This approach is helpful where, for example, the mobile computing device lacks sufficient resources to perform ASR processing locally.

In other examples of the act 1114, the event handler generates a distinct media file for each section (e.g., ROS, paragraph, sentence, or other section) that includes audio entries for that section. In these examples, the event handler may transmit media files with audio entries excluded from the set of targeted audio entries to a (local or remote) ASR system to generate ASR transcripts. Further, in these examples, the event handler may transmit media files with audio entries included in the set of targeted audio entries to a remote transcription system to generate ASR transcripts that are reviewed by humans. This approach is helpful where, for example, network bandwidth is a concern and the mobile computing device possesses sufficient resources to perform some of the operations recited above locally.

In other examples of the act 1114 in which the event handler generates a distinct media file for each EHR section, the event handler may transmit all media files to a (local or remote) ASR system to generate ASR transcripts. Further, in these examples, the event handler may transmit media files, ASR transcripts, and related information for audio entries included in the set of targeted audio entries to a remote transcription system for review and correction by human editors. Additionally or alternatively, in these examples, the event handler may transmit media files, ASR transcripts, and/or related information for audio entries excluded in the set of targeted audio entries to the remote transcription system for additional processing (e.g., expansion of word macros, client editing, etc.). This approach is helpful where, for example, the remote transcription system is resource constrained and distributed ASR processing benefits the efficiency of the overall system.

If the order transcription control 1034 was not selected, in act 1108 the event handler determines whether the backward navigation control 528 was selected. If the event handler determines that the backward navigation control 528 was selected, the event handler returns to the interface process 900 described above with reference to FIG. 9. Otherwise, the event handler returns to the act 1102, and the interface process 1100 reiterates.

In some embodiments, when presenting the transcription ordering screen 1000, the event handler creates section JSON objects to indicate whether each is included or excluded from the set of audio entries for human view. These section JSON objects may be transmitted along with the media file(s) in response to selection of the order transcription control 1034. For example, the section JSON may be [{“section”: “HPI”, “service_level”: “reviewed”},{“section”: “PE”, “service_level”: “asr_only”} . . . ].

In some embodiments, prior to presenting the transcription ordering screen 1000, the event handler is configured to generate an ASR transcript (e.g., via the ASR system interface 208). In a local, real-time ASR implementation, generation of the ASR transcript can be rapid, on the order of seconds. In a remote, batch ASR implementation, generation of the ASR transcript can take about the duration of the full dictation (e.g. a few minutes). Often, a batch ASR implementation, which is typically more accurate, fits well with a health care provider's workflow, where the health care provider may perform a number of dictations in sequence before reviewing the status of each one. In these embodiments, the transcription ordering screen 1000 presents indicators of confidence in the correctness of the ASR translation (e.g. those reflected in the ASR_cost described in the Electronic Transcription Job Market patent). The event handler may be configured to present confidence at different “levels”, for example, at the entire encounter level (an “overall confidence”), at the section level, at the sentence level, at the phrase level, or even at the word level. The event handler may indicate confidence using a variety of metaphors within the transcription ordering screen 1000, e.g. using text and/or background coloring, hover-over pop-ups with “estimate accuracy” number, font changes, etc.

In some embodiments, the event handler automatically selects and/or deselects section selection controls depending on one or more confidence thresholds associated with the EHR sections. In these embodiments, the event handler compares a confidence indicator for each section with a threshold confidence for the section. Where the confidence indicator exceeds the threshold confidence, the event handler automatically deselects its associated section selection control. Where the confidence indicator does not exceed the threshold confidence, the event handler automatically selected its associated section selection control. The event handler may be configured to execute these comparisons at any level for which confidence indicators are calculated by ASR processing.

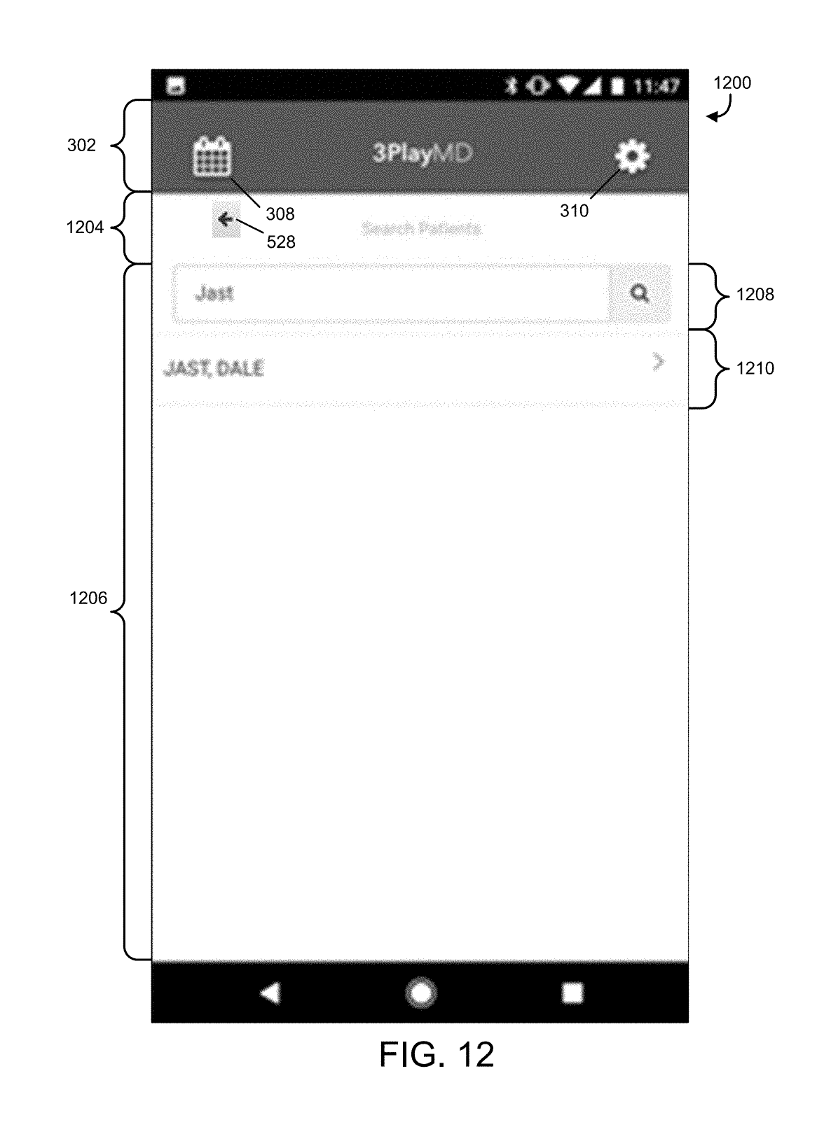

FIG. 12 illustrates the patient search screen 1200 as presented by at least one embodiment of the flexible recording interface. As shown in FIG. 12, the patient search screen 1200 is sized and arranged for the display of the mobile computing device. In some embodiments, prior to presenting the patient search screen 1200, the event handler interoperates with a remote EHR system (via the transcript database system interface 204) to retrieve transcript data representative of historical transcripts for one or more patient (e.g., patients with appointments scheduled for the date selected in the calendar control 308). This transcript data may be stored in the transcript data store 124 and may include data representative of patient names, identifiers, transcript text, and corresponding audio entries.

The patient search screen 1200 includes some elements similar to the elements of the recording screen 700 (e.g., the app header 302, the calendar control 308, the settings control 310, and the backward navigation control 528). These elements of the patient search screen 1200 are structured and function like the elements of the recording screen 700.

The patient search screen 1200 is segmented into the app header 302, a screen header 1204, and a body 1206. The screen header 1204 includes the backward navigation control 528 and a title of the screen “Search Patients.” The body 1006 includes patient search control 1208 and a patient selection control 1210. As shown in FIG. 12, the patient search control 1208 accepts user input specifying a patient search string. The search string may include at least a portion of a patient's name or other patient identifier. As shown in FIG. 12, the patient selection control 1210 presents names of patients who match the search string.

During the presentation of the patient search screen 1200, the event handler interoperates with the user interface component to execute an interface process 1300 that is illustrated in FIG. 13. As shown in FIG. 13, the interface process 1300 starts in act 1302 with the event handler receiving input specifying a patient search string via the patient search control 1208.

In act 1304, the event handler searches transcript data (e.g., the locally stored transcript data store 124) for patient identifiers (e.g., names) that match the patient search string. This searching may include, for example, accessing an inverted index stored in the transcript data that is keyed on patent names and identifying one or more patient names in the inverted index that include the patient searching string. In act 1306, the event handler presents results of the search via one or more patient selection controls, such as the patient selection control 1210.

In act 1308, the event handler receives a selection of an element of the patient search screen 1200. In act 1310, the event handler determines whether the backward navigation control 528 was selected. If the event handler determines that the backward navigation control 528 was selected, the event handler returns to the interface process 400 described above with reference to FIG. 4. Otherwise, in act 1312 the event handler determines whether the patient selection control 1210 was selected. If so, in act 1314 the event handler presents a patient transcripts screen (e.g., the patient transcripts screen 1400 described further below). Otherwise, the event handler returns to the act 1302 to receive another patient search string.

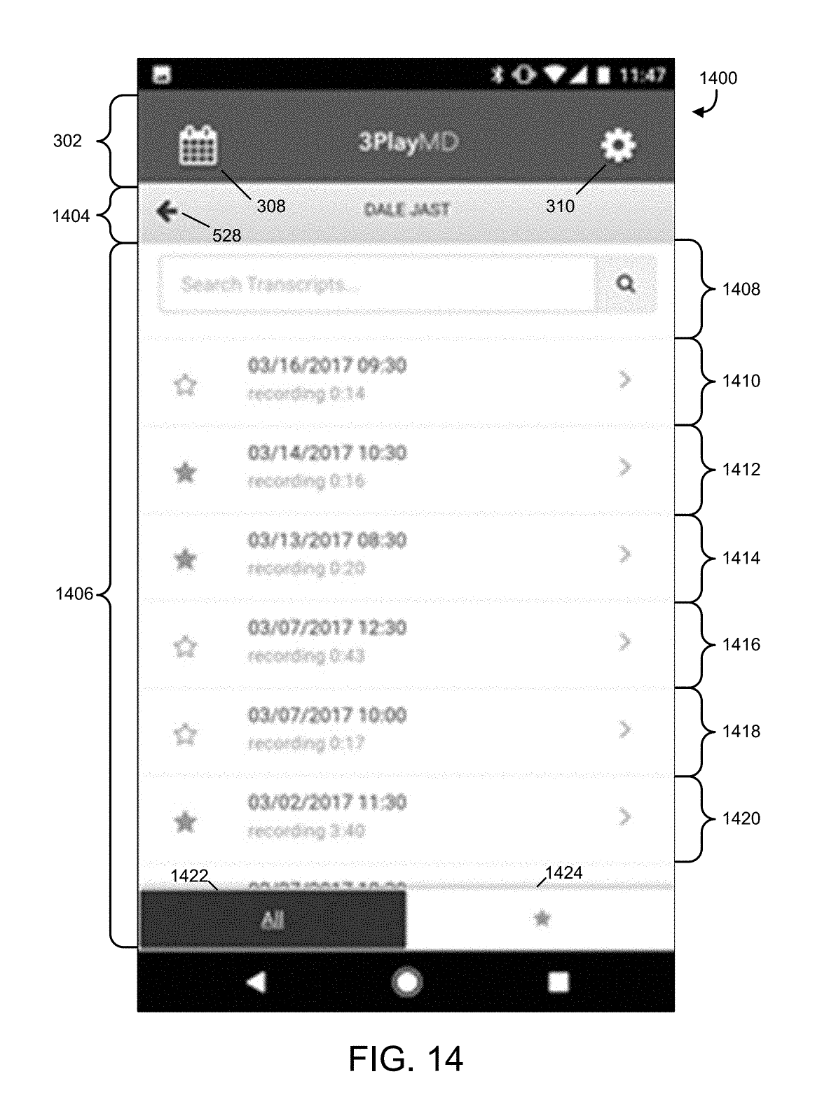

FIG. 14 illustrates the patient transcripts screen 1400 as presented by at least one embodiment of the flexible recording interface. As shown in FIG. 14, the patient transcripts screen 1400 is sized and arranged for the display of the mobile computing device. The patient transcripts screen 1400 includes some elements similar to the elements of the recording screen 700 (e.g., the app header 302, the calendar control 308, the settings control 310, and the backward navigation control 528). These elements of the patient transcripts screen 1400 are structured and function like the elements of the recording screen 700.

The patient transcripts screen 1400 is segmented into the app header 302, a screen header 1404, and a body 1406. The screen header 1404 includes the backward navigation control 528 and the name of the patient associated with the selected patient selection control (e.g., the patient selection control 1210). The body 1406 includes patient transcripts search control 1408, patient transcript selection controls 1410-1420, and bookmark filter controls 1422 and 1424. As shown in FIG. 12, the patient transcripts search control 1408 accepts user input specifying a patient transcript search string. The search string may include at least a portion of a date and/or time of an appointment that the patient transcript documents or other patient transcript identifier. As shown in FIG. 14, each of the patient transcripts selection controls 1410-1420 presents dates, times, and media durations for patient transcripts of appointments that match the search string.

Returning to FIG. 13, in act 1316 the event handler receives a selection of an element of the patient transcripts screen 1400. In act 1318, the event handler determines whether the backward navigation control 528 was selected. If the event handler determines that the backward navigation control 528 was selected, in act 1324 the event handler presents the search patents screen. Otherwise, in act 1320 the event handler determines whether one of the bookmark filter controls 1422 and 1424 was selected.

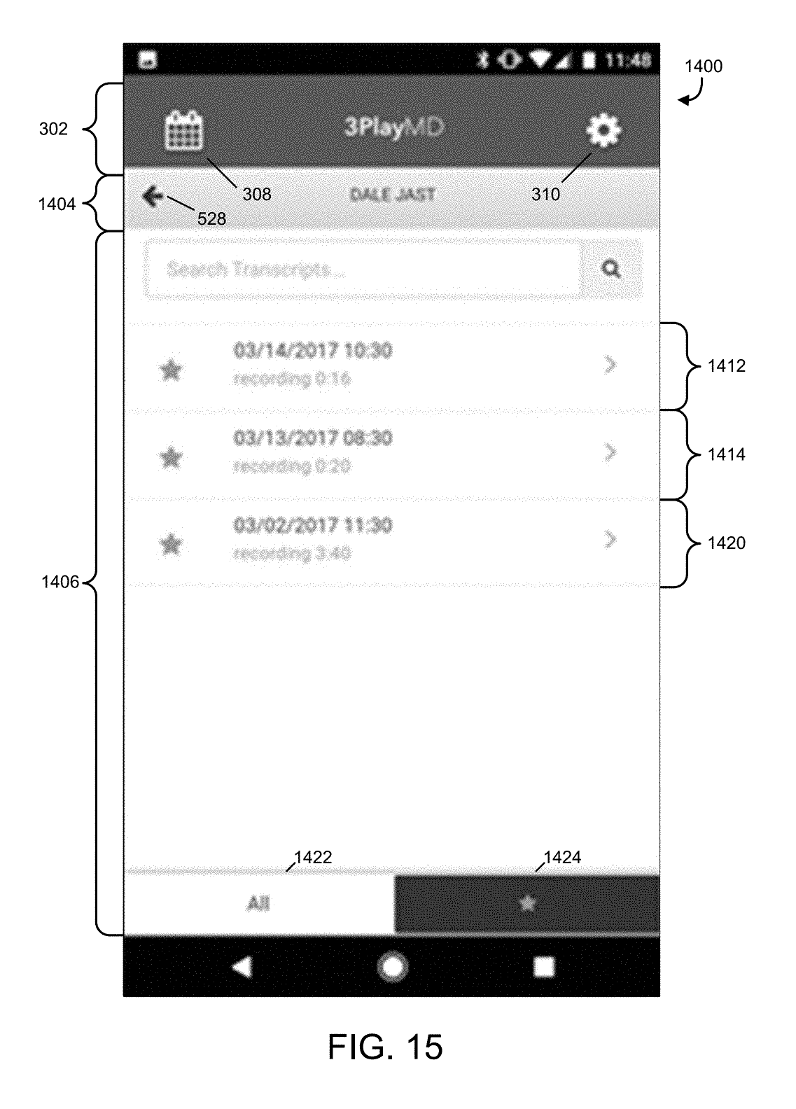

If one of the bookmark filter controls 1422 and 1424 was selected, in act 1326 the event handler adjusts the patient transcripts displayed by the patient transcripts screen 1400. More specifically, if the bookmark filter 1422 was selected, the event handler presents all of the patient transcripts for a selected patient. FIG. 14 illustrates one such example. If the bookmark filter 1422 was selected, the event handler presents patient transcripts for the selected patient that have been bookmarked. FIG. 15 illustrates one such example.

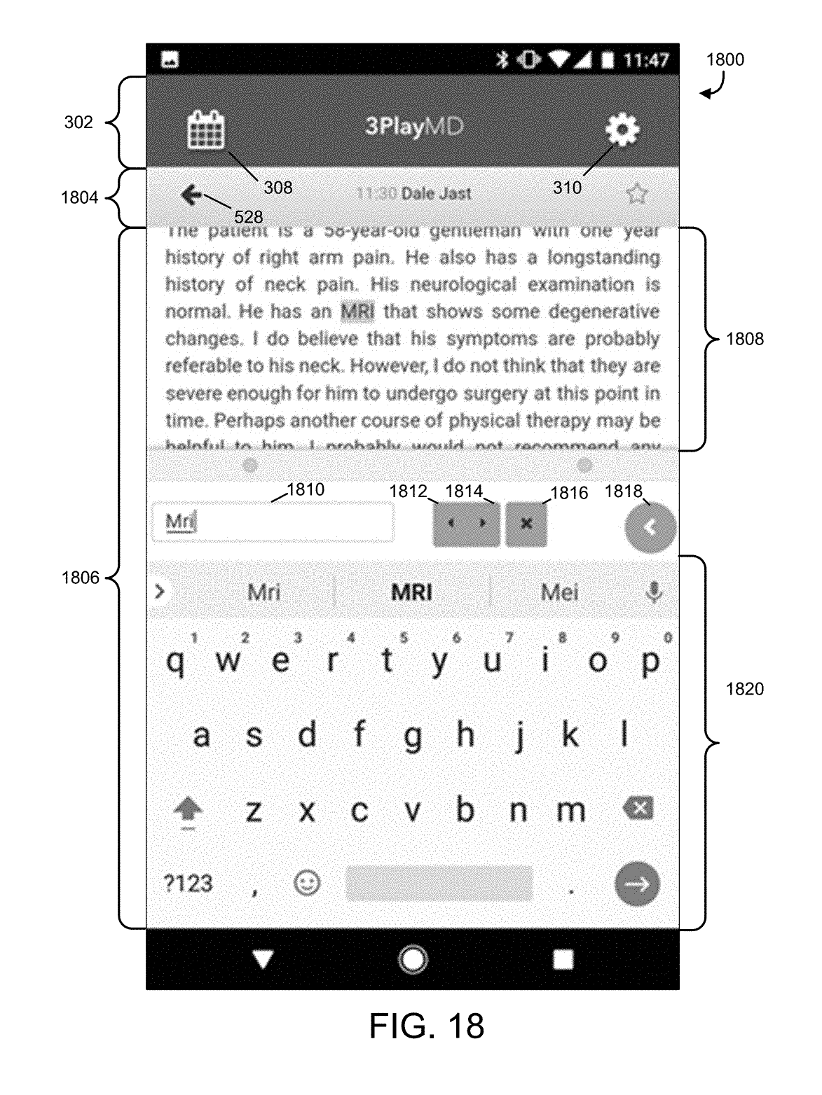

In act 1322 the event handler determines whether one of the patient transcript selection controls 1410-1420 was selected. If so, in act 1328 the event handler presents a transcript screen (e.g., the transcript screen 1600 described further below) and proceeds to an interface process 1800 described below with reference to FIG. 18. Otherwise, the event handler returns to the act 1316 to receive another selection.

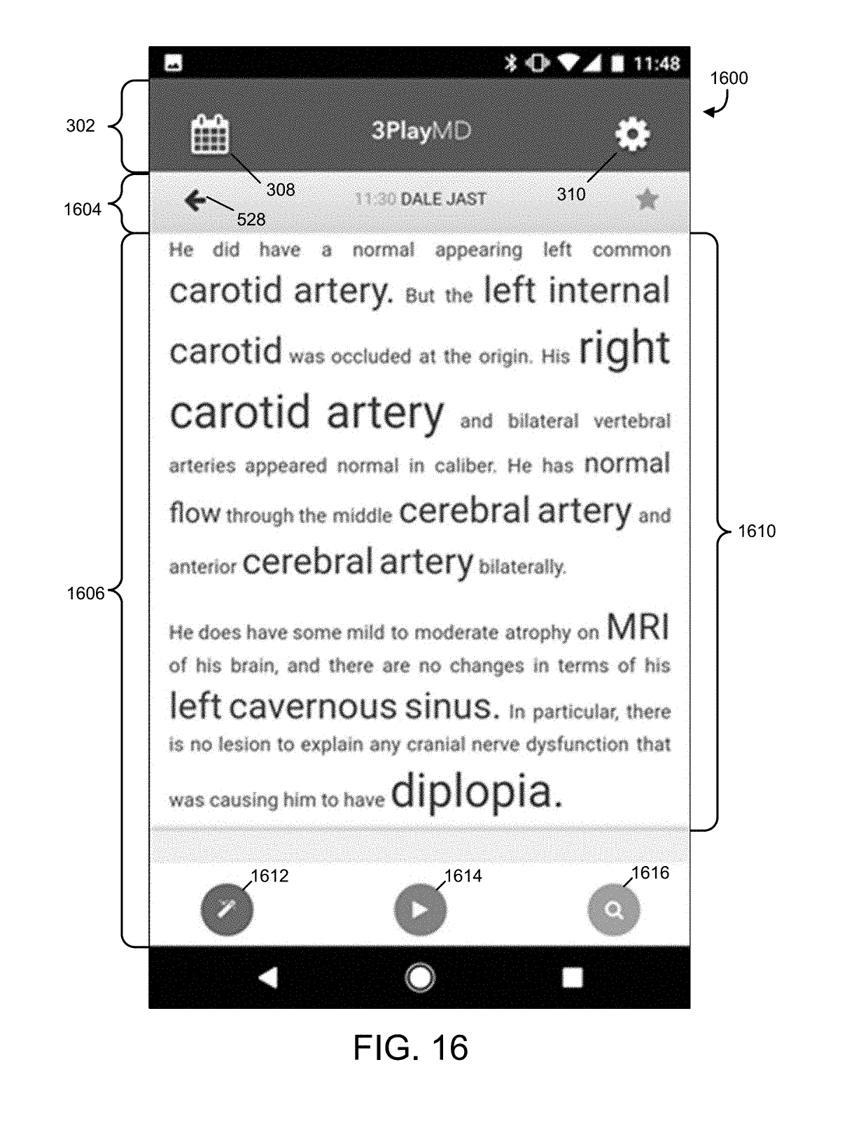

FIG. 16 illustrates the transcript screen 1600 as presented by at least one embodiment of the flexible recording interface. The transcript screen 1600 includes some elements similar to the elements of the recording screen 700 (e.g., the app header 302, the calendar control 308, the settings control 310, and the backward navigation control 528). These elements of the transcript screen 1600 are structured and function like the elements of the recording screen 700.

The transcript screen 1600 is segmented into the app header 302, a screen header 1604, and a body 1606. The screen header 1604 includes the backward navigation control 528 and the name of the patient and the time of the appointment documented by the transcript being viewed. The body 1606 includes transcript view control 1610, a magic wand control 1612, a play audio control 1614, and a keyword search control 1616. As shown in FIG. 16, the magic wand control 1612 is selected, which causes the transcript view control 1610 to present transcript text in using weighted list motif that emphasizes medical terminology.

As shown in FIG. 16, the transcript screen 1600 is sized and arranged for the display of the mobile computing device. For example, by presenting keywords that are sized in proportion to their importance, the transcript screen 1600 enables a user to easily and quickly scroll through the transcript to find key terms, and then read or playback from that point in the audio to interpret the surrounding context.

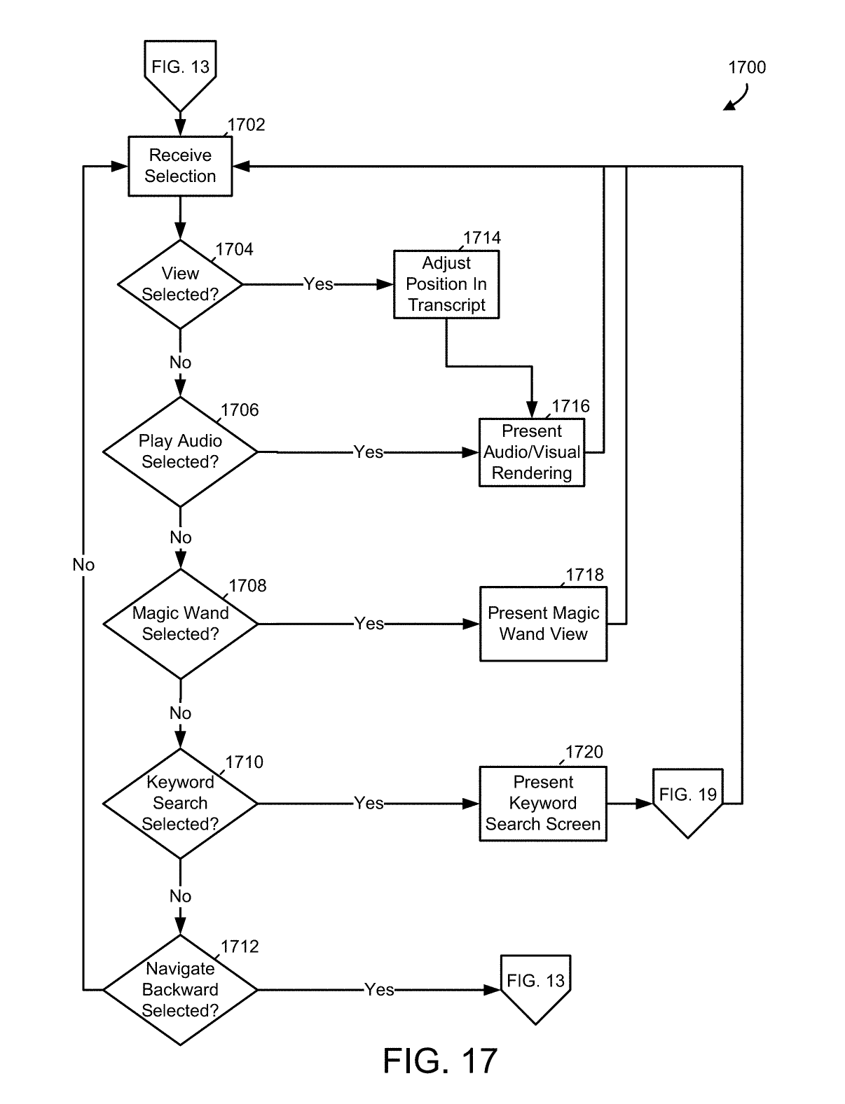

During the presentation of the transcript screen 1600, the event handler interoperates with the user interface component to execute an interface process 1700 that is illustrated in FIG. 17. As shown in FIG. 17, the interface process 1700 starts in act 1702 with the event handler receiving a selection of an element of the transcript screen 1600.

In act 1704, the event handler determines whether the transcript view control 1610 was selected. If so, in act 1714 the event handler highlights a word nearest the selected position within the transcript view control 1610 and proceeds to act 1716. The highlighted word serves as a starting position for playback of the transcript as described further below with reference to the act 1716.

In act 1706, the event handler determines whether the play audio control 1614 was selected. If so, in the act 1716 the event handler steps through the transcript text word by word—concurrently presenting an audio rendering of each word while highlighting the word within the transcript view control—until some other element of the transcript screen 1600 is selected. In executing the act 1716, the event handler starts at a default position within the transcript text (e.g., the beginning) unless another position was previously selected (e.g., within the act 1704 described above).

If the play audio control 1614 was not selected, in act 1708 the event handler determines whether the magic wand control 1612 was selected. If so, in act 1718 the event handler presents a magic wand view of the transcript screen 1600, which is illustrated in FIG. 16. Otherwise, in act 1710 the event handler determines whether the keyword search control 1616 was selected. If so, in act 1720 the event handler presents a keyword search screen (e.g., the keyword search screen 1800 described further below) and proceeds to an interface process 1900 described below with reference to FIG. 19.

If the event handler determines that the keyword search control 1616 was not selected, in act 1712 determines whether the backward navigation control 528 was selected. If the event handler determines that the backward navigation control 528 was selected, the event handler returns to the interface process 1300 described above with reference to FIG. 13. Otherwise, the event handler returns to the act 1702, and the interface process 1700 reiterates.

FIG. 18 illustrates the keyword search screen 1800 as presented by at least one embodiment of the flexible recording interface. As shown in FIG. 18, the keyword search screen 1800 is sized and arranged for the display of the mobile computing device. The keyword search screen 1800 includes some elements similar to the elements of the recording screen 700 (e.g., the app header 302, the calendar control 308, the settings control 310, and the backward navigation control 528). These elements of the keyword search screen 1800 are structured and function like the elements of the recording screen 700.

The keyword search screen 1800 is segmented into the app header 302, a screen header 1804, and a body 1806. The screen header 1804 includes the backward navigation control 528 and the name of the patient and the time of the appointment documented by the transcript being viewed. The body 1806 includes transcript view control 1808, a keyword control 1810, transcript navigation controls 1812 and 1814, keyboard controls 1816-1820. As shown in FIG. 18, the keyword control 1810 includes the keyword “Mri” and the transcript text “MRI” is highlighted in the transcript view control 1808.

During the presentation of the keyword search screen 1800, the event handler interoperates with the user interface component to execute an interface process 1900 that is illustrated in FIG. 19. As shown in FIG. 19, the interface process 1900 starts in act 1902 with the event handler receiving a selection of an element of the keyword search screen 1800.

In act 1904, the event handler determines whether one of the keyboard controls 1816-1820 was selected. If so, in act 1920 the event handler adjusts the content of the keyword control 1810. More specifically, if the keyboard control 1816 was selected, the event handler clears all text from the keyword control 1810. If the keyboard control 1818 was selected, the event handler deletes the letter next to the cursor in the keyword control 1810. If any key on the keyboard control 1820 was selected, the event handler enters that letter, emoji, etc. in the keyword control 1810 to the right of the cursor.

If the event handler determines that one of the keyboard controls 1816-1820 was not selected, in act 1906 the event handler determines whether one of the transcript navigation controls 1812 and 1814 was selected. If so, in act 1912 the event handler adjusts the presentation of the transcript text in the transcript view control 1808. More specifically, if the transcript navigation control 1812 was selected, the event handler navigates within the transcript to an occurrence of the keyword listed in the keyword control 1810 previous to the currently presented occurrence. If the transcript navigation control 1814 was selected, the event handler navigates within the transcript to an occurrence of the keyword listed in the keyword control 1810 subsequent to the currently presented occurrence.

If the event handler determines that one of the transcript navigation controls 1812 and 1814 was not selected, in act 1908 the event handler determines whether the backward navigation control 528 was selected. If the event handler determines that the backward navigation control 528 was selected, the event handler returns to the interface process 1700 described above with reference to FIG. 17. Otherwise, the event handler returns to the act 1902, and the interface process 1900 reiterates.

In some embodiments, during the presentation of the transcript screen 1600 or the keyword search screen 1800, the event handler executes one or more voice macros by interoperating with a voice macro processor (e.g., the voice macro processor 210). For instance, within an internal-medicine/family-practice, where the review of systems and physical examination sections are heavily utilized, the event handler may execute a voice macro to replace trigger text (e.g., “Please use my normal physical exam”) with the following text.

### Physical Examination:

VITAL SIGNS: Temperature tactilely afebrile, blood pressure XX/YY, weight ZZZ, height A feet B inches.
GENERAL: The patient is a well-developed, well-nourished male in no acute distress, A&O x3.
HEENT: Normocephalic, atraumatic. Extraocular muscles are intact. Conjunctivae pink. Sclerae anicteric. Pupils equal, round and reactive to light. Fundi sharp with no exudate or hemorrhages. Tympanic membranes clear. Nasal mucosa normal. Septum midline. No purulent exudates. Buccal mucosa moist, no lesions. No caries, no pharyngeal injection, no exudate.
NECK: Supple, no carotid bruits, no adenopathy. Thyroid normal size, shape and contour.
CARDIAC: Regular rate and rhythm. No murmurs, rubs or gallops.
LUNGS: Clear to auscultation bilaterally. No wheezes, rales or rhonchi.
ABDOMEN: Bowel sounds present, nontender, nondistended. No hepatosplenomegaly. No masses detected. No deformity, no CVA tenderness.
EXTREMITIES: No cyanosis, clubbing or edema. No varicosities noted. DP pulses+2 in bilateral extremities.
MUSCULOSKELETAL: Normal gait and grossly nonfocal.
NEUROLOGIC: Cranial nerves II through XII grossly intact. Sensation intact to fine touch bilaterally and to vibration in bilateral lower extremities. Deep tendon reflexes equal bilaterally. Babinski's equivocal. Motor strength 5+ throughout.
DERMATOLOGIC: No exanthems, no suspicious lesions. The patient is noted to have skin tags around the neck.

As shown in the VITAL SIGNS sub-section above, there are variables which may be efficiently filled in (i.e., XX/YY, ZZZ, A, and B).

In another example directed to a cardiology practice, the event handler may execute a voice macro to replace trigger text (e.g., “Insert my standard Discharge instructions”) with the following text.

DISCHARGE INSTRUCTIONS: Since the patient had generalized deconditioning, the patient was advised home PT, OT and that was arranged for the patient.
DISCHARGE DIET: Cardiac diet.
DISCHARGE ACTIVITY: Resume activity as tolerated.
And, many Operative/Procedure notes have standard summaries of the procedure (believe it or not!), e.g.:

In another example, a Pain medicine procedure note, the event handler may execute a voice macro to replace trigger text (e.g., “Insert my normal caudal epidural steroid injection with fluoroscopy”) with the following text.

### Procedure:

1) Caudal epidural steroid injection
2) Fluoroscopic needle guidance

### REASON FOR PROCEDURE: XXX

PHYSICIAN: Dr. Howard
MEDICATIONS INJECTED: 2 mL of Depo-Medrol (80 mg) and 3 mL of sterile, preservative-free normal saline
LOCAL ANESTHETIC INJECTED: 7 mL of 1% lidocaine

### SEDATION MEDICATIONS: None

### ESTIMATED BLOOD LOSS: None.

### COMPLICATIONS: None

TECHNIQUE: Time-out was taken to identify the correct patient, procedure and side prior to starting the procedure. Lying in the prone position, the patient was prepped and draped in sterile fashion using DuraPrep and a fenestrated drape. Appropriate landmarks were determined using a lateral fluoroscopic image. Local anesthetic was given by raising a wheal and going down to the hub of a 27-gauge 1.25-inch needle. A 22-gauge, 3.5-inch Quincke needle was introduced through the sacral hiatus. The needle was advanced cephalad to just caudal to the inferior sacroiliac joint line. Omnipaque 240 was injected to confirm placement in the appropriate epidural space, and to show that there was no run-off. The medication was then injected slowly. The procedure was completed without complications and was tolerated well. The patient was monitored after the procedure. The patient (or responsible party) was given post-procedure and discharge instructions to follow at home. The patient was discharged in stable condition. A follow up appointment was made.

So, in this case, the health care provider would record further audio entries to indicate the reason for the procedure (XXX). But, otherwise, the report would be entirely filled in by recordation of the trigger text.

In some embodiments, the event handler is configured to execute voice macros to create coded diagnoses and orders according to a user's preferences. For example, in these embodiments, trigger text for a repeated task such as “please use my strep throat standard” can generate expansion text in a standard Assessment and Plan section, as well as create draft billing codes for a strep throat test and a prescription based on the health care provider's preferences of antibiotic medication. The voice macros may further encode logic to determine dosage requirements based on factors such as the age and weight of the patient. Draft orders, such as these, are presented for review by the health care provider in the EHR after the final transcripts are transmitted and imported into the EHR system via, for example, the transcript database system interface 204 or the transcript database system interface 2240 described further below with reference to FIG. 22.

In some embodiments, during the presentation of the transcript screen 1600 or the keyword search screen 1800, the event handler provides association controls that enable a user to associate metadata with portions of the transcript text, as described in the Metadata Media Associator patent. In these embodiments, where the event handler receives user input selecting an association control, the event handler interoperates with the user interface component and an association engine (e.g., the association engine 212) to create an association between a selected portion of transcript text and metadata. In these embodiments, the user interface component is configured to present indicators of such associations within the transcript screen 1600 and/or the keyword search screen 1800 (e.g., during playback of the transcript). These indicators may include, for instance, a tooltip presented while a cursor hovers over the relevant portion of the transcript text.

In one example, if the transcript text refers to an X-Ray, an association may be inserted between the transcript text and a digital image of the X-Ray. In another example, if the transcript text refers to an order for a laboratory test an association may be inserted between the transcript text and the relevant SNOMED code. Later, when the laboratory test is completed, the associations reference updated, potentially in real-time, the results of the laboratory test. In some embodiments, where the event handler determines that the associated metadata refers to text, the event handler may insert the metadata directly into the transcript as text prior to transmitting the transcript to a transcription system or an EHR system.

Thus, using these associative features, a user (e.g. a medical scribe) can help document a patient encounter by associating billing codes and other items, such as X-Rays, lab results and the like, with EHR entries documenting a patient encounter that are generated by a doctor. By tying these items to the EHR entries, a doctor reviewing all of the encounter records at the end of the day is able to listen back to their voice, along with those billing codes and other items, to ensure 100% accuracy of what the scribe documented. This approach improves accuracy by providing an efficient double-checking process.

In some embodiments, the event handler is configured to automate metadata association. In these embodiments, the event handler may leverage keyword extraction to increase the efficiency of association operations for the user. For instance, if the targeted keyword (e.g., “PSA Test”) was identified via keyword extraction, the event handler may be configured to present a dialog to order the test. Where the user responds in the affirmative to the dialog, the event handler may insert an order for the test into the transcript text and EHR.

FIG. 20 illustrates the transcript defaults screen 2000 as presented by at least one embodiment of the flexible recording interface. As shown in FIG. 20, the transcript defaults screen 2000 is sized and arranged for the display of the mobile computing device. The transcript defaults screen 2000 includes some elements similar to the elements of the recording screen 700 (e.g., the app header 302, the calendar control 308, the settings control 310, and the backward navigation control 528). These elements of the transcript defaults screen 2000 are structured and function like the elements of the recording screen 700.

The transcript defaults screen 2000 is segmented into the app header 302, a screen header 2004, and a body 2006. The screen header 2004 includes the backward navigation control 528 and a title of the screen, “Transcript Review Defaults.” The body 2006 includes section selection controls 2008-2020. As shown in FIG. 20, the section selection controls 2012, 2014, and 2020 are selected, as indicated by the checkmark displayed in each.

During the presentation of the transcript defaults screen 2000, the event handler interoperates with the user interface component to execute an interface process 2100 that is illustrated in FIG. 21. As shown in FIG. 21, the interface process 2100 starts in act 2102 with the event handler receiving a selection of an element of the transcript defaults screen 2000.

In act 2104, the event handler determines whether one of the section selection controls 2008-2020 was selected. If so, in act 2108 the event handler modifies the default set of EHR sections including audio entries targeted for human review. More specifically, if the section selection control 2008 was selected, the event handler excludes all of the EHR sections listed in the body 2006 from the default set of EHR sections. If the section selection control 2010 was selected, the event handler includes all of the EHR sections listed in the body 2006 in the default set of EHR sections. If the section selection control 2012 was selected, the event handler toggles (e.g., excludes if currently included or includes if currently excluded) the HPI section relative to the default set of EHR sections. If the section selection control 2014 was selected, the event handler toggles the ROS section relative to the default set of EHR sections. If the section selection control 2016 was selected, the event handler toggles the PE section relative to the default set of EHR sections. If the section selection control 2018 was selected, the event handler toggles the Discussion section relative to the default set of EHR sections. If the section selection control 2020 was selected, the event handler toggles the Assessment and Plan section relative to the default set of EHR sections.

In some embodiments, the effect of the specific section selection controls (i.e., section selection controls 2012-2020) overrides the effect of the broader section selection controls (i.e., section selection controls 2008 and 2010). In these embodiments, where a broader section selection control is selected, the specific section selection controls indicate the inclusion or exclusion effects of the broader section selection controls. However, the specific section selection controls can be subsequently selected to override the effect of the broader selection control. FIG. 20 illustrates one example of this feature. As shown in FIG. 20, the section selection control 2010 was initially selected to include all of the EHR sections, but section selection controls 2016 and 2018 were subsequently selected to toggle (here, to exclude) the PE and Discussion sections from the default set of EHR sections.

If none of the section selection controls 2008-2020 was selected, in act 2106 the event handler whether the backward navigation control 528 was selected. If the event handler determines that the backward navigation control 528 was selected, the event handler returns to the interface process 400 described above with reference to FIG. 4. Otherwise, the event handler returns to the act 2102, and the interface process 2100 reiterates.

In some embodiments, the section selection controls 2012-2020 may each include a confidence selection control indicates a threshold confidence for each section. As described above with reference to FIG. 10, the threshold confidence may be used in some embodiments to include or exclude audio entries from the set of audio entries for human review. In these embodiments, the event handler is configured to adjust the threshold confidence in response to receiving a selection of the confidence selection control to reflect a value input by the user. The event handler may render values of the threshold confidence within the confidence selection control as text boxes, sliders, or other types of controls.

### Transcription System

Various embodiments implement a transcription system using one or more computer systems. FIG. 22 illustrates one of these embodiments, a transcription system 2200. As shown, FIG. 22 includes a server computer 2202, client computers 2204, 2206, and 2208, a transcript database system 2238, a customer 2210, an editor 2212, an administrator 2214, networks 2216, 2218 and 2220, and an automatic speech recognition (ASR) device 2222. The server computer 2202 includes several components: a customer interface 2224, an editor interface 2226, a system interface 2228, an administrator interface 2230, a transcript database system interface 2240, a market engine 2232, a market data storage 2234, and a media file storage 2236.

As shown in FIG. 22, the system interface 2228 exchanges (i.e. sends or receives) media file information with the ASR device 2222. The transcript database system interface 2240 exchanges information the with transcript database system 2238. The customer interface 2224 exchanges information with the client computer 2204 via the network 2216. The editor interface 2226 exchanges information with the client computer 2206 via the network 2218. The networks 2216, 2218 and 2220 may include any communication network through which computer systems may exchange information. For example, the network 2216, the network 2218, and the network 2220 may be a public network, such as the internet, and may include other public or private networks such as LANs, WANs, extranets and intranets.

Information within the transcription system 2200, including data within the market data storage 2234 and the media file storage 2236, may be stored in any logical construction capable of holding information on a computer readable medium including, among other structures, file systems, flat files, indexed files, hierarchical databases, relational databases or object oriented databases. The data may be modeled using unique and foreign key relationships and indexes. The unique and foreign key relationships and indexes may be established between the various fields and tables to ensure both data integrity and data interchange performance. In one embodiment, the media file storage 2236 includes a file system configured to store media files and other transcription system data and acts as a file server for other components of the transcription system. In another embodiment, the media file storage 2236 includes identifiers for files stored on another computer system configured to serve files to the components of the transcription system.

Information may flow between the components illustrated in FIG. 22, or any of the elements, components and subsystems disclosed herein, using a variety of techniques. Such techniques include, for example, passing the information over a network using standard protocols, such as TCP/IP or HTTP, passing the information between modules in memory and passing the information by writing to a file, database, data store, or some other non-volatile data storage device. In addition, pointers or other references to information may be transmitted and received in place of, in combination with, or in addition to, copies of the information. Conversely, the information may be exchanged in place of, in combination with, or in addition to, pointers or other references to the information. Other techniques and protocols for communicating information may be used without departing from the scope of the examples and embodiments disclosed herein.

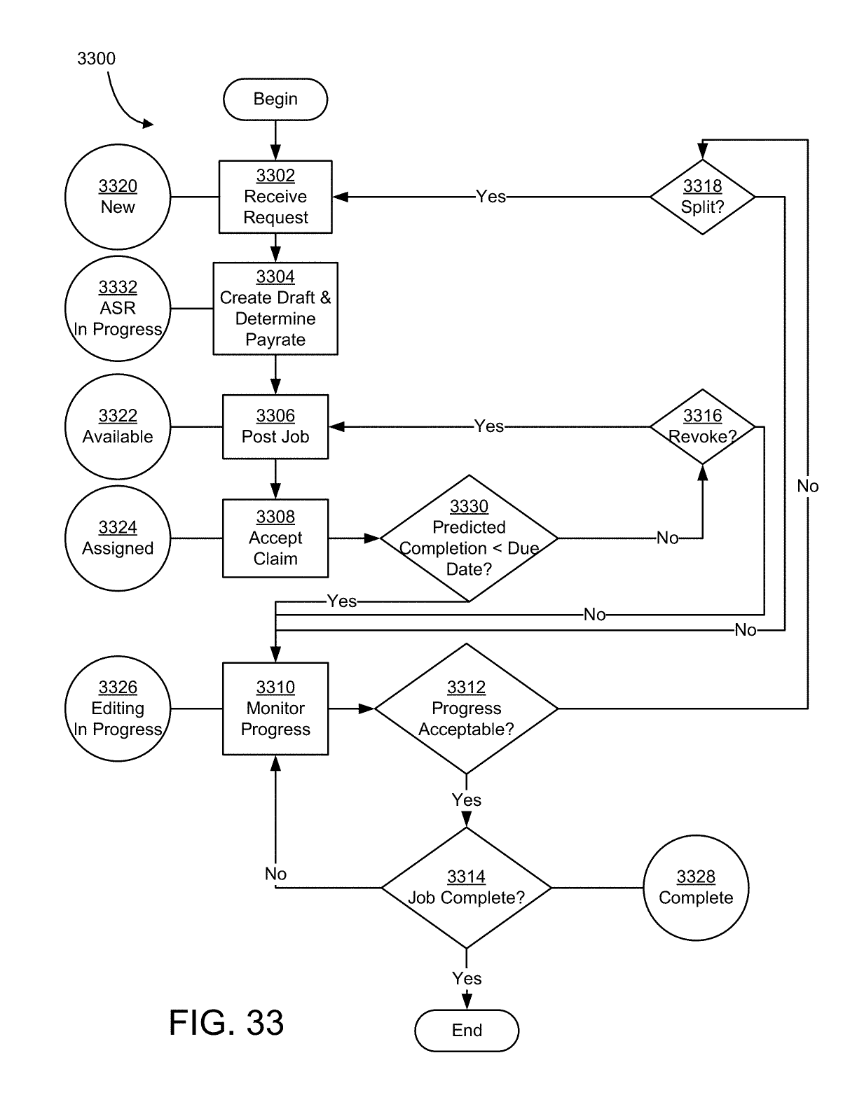

One goal of the transcription system 2200 is to receive media files from customers and to provide both final and/or intermediate transcriptions of the content included in the media files to the customers. One vehicle used by the transcription system 2200 to achieve this goal is a transcription job. Within the transcription system 2200, transcription jobs are associated with media files and are capable of assuming several states during processing. FIG. 33 illustrates an exemplary process 3300 during the execution of which a transcription job assumes several different states.

As shown in FIG. 33, the process 3300 begins when the transcription system 2200 receives transcription request information that identifies a media file to transcribe in act 3302. The transcription request information may also include delivery criteria that specifies a schedule (e.g., one or more delivery times), quality levels, or other criteria defining conditions to be satisfied prior to delivery of transcription products. For media files documenting patient encounters for the EHR, the transcription request information may also include audio entry and section JSON objects as described above. In some embodiments, the transcription system 2200 receives the transcription request information and the media file via an upload from a mobile recording application, such as the mobile recording application 118, a customer interface, such as the customer interface 2224, or as a result of a previously received media file being split, per act 3318 below. Upon receipt of the transcription request information and the media file, the transcription system 2200 creates a job, associates the job with the media file, and sets the job to a new state 3320.

In some embodiments, in the act 3302 the transcription system 2200 processes the section JSON objects included in the transcription request information and creates a single editing and/or QA job for a media file documenting a patient encounter for the EHR. In other embodiments in the act 3302 the transcription system 2200 processes the section JSON objects and creates multiple, distinct editing and/or QA jobs—one for each section selected for human review.

In act 3304, the transcription system 2200 sets the job to an ASR in progress state 3332, generates draft transcription information, and determines a pay rate for the job. When executing the act 3304, some embodiments track completion percentage of the draft transcription during ASR processing. Record of completion percentage is used to execute subsequent delivery processes where ASR processing is not complete due to the schedule or interruption by another delivery request. Further, these embodiments compute one or more metrics that characterize the quality of the draft transcription. Draft transcriptions may be full transcriptions or partial transcriptions (where ASR processing is not completed). Some embodiments incorporate information descriptive of the completion percentage and quality metrics into the draft transcription information.

In act 3306, the transcription system 2200 posts the job, making the job available for editors to claim, and sets the job to an available state 3322. Jobs in the available state correspond to draft transcriptions that have completed full or partial ASR processing. As described further below, in some embodiments in accord with FIG. 33, the transcription system 2200 monitors the due dates and times of available jobs and, if necessary, alters the pay rate (or other job characteristics) of the available jobs to ensure the available jobs are completed by the due date and time.

In act 3308, the transcription system 2200 accepts an offer by an editor to claim the job and sets the job to an assigned state 3324. In the illustrated embodiment, jobs in the assigned state 3324 are not available for claiming by other editors. In act 3330, the transcription system 2200 determines whether the predicted completion date and time for the job, as assigned, occurs before the due date and time. If so, the transcription system 2200 executes act 3310. Otherwise the transcription system 2200 executes act 3316.

In the act 3316, the transcription system 2200 determines whether to revoke the job. If so, the transcription system executes the act 3306. Otherwise, the transcription system 2200 executes the act 3310.

In the act 3310, the transcription system 2200 records and monitors actual progress in transcribing the media file associated with the job, as the progress is being made by editors. Also in the act 3310, the transcription system 2200 sets the job to an editing in progress state 3326. In the act 3312, the transcription system 2200 determines whether the job is progressing according to schedule. If so, the transcription system executes act 3314. Otherwise, the transcription system executes act 3318.

In the act 3318, the transcription system 2200 determines whether to split the media file associated with the job into multiple media files. For example, the transcription system may split the media file into one segment for any work already completed and into another segment for work yet to be completed. This split may enable the transcription system 2200 to further improve the quality on a segment by segment basis. For example, a segment which has been edited may be split from other segments so that the edited segment may proceed to quality assurance (QA). Thus splitting the media file may enable the transcription system to provide partial but progressive delivery of one or more transcription products to customers. If the transcription system 2200 splits the media file, the transcription system 2200 stores the edited, completed segment and executes the act 3302 for any segments that include content not completely transcribed. If, in the act 3318, the transcription system 2200 determines to not split the media file, the transcription system 2200 executes the act 3310.

In the act 3314, the transcription system 2200 determines whether the content of the media file associated with the job is completely transcribed. If so, the transcription system 2200 stores the edited, complete transcription and sets the state of the job to a complete state 3328, and the process 3300 ends. Otherwise, the transcription system 2200 executes the act 3310.

In some embodiments, completed transcriptions may be the subject of other jobs, such as QA jobs, as described further below. Components included within various embodiments of the transcription system 2200, and acts performed as part of the process 3300 by these components, are described further below.

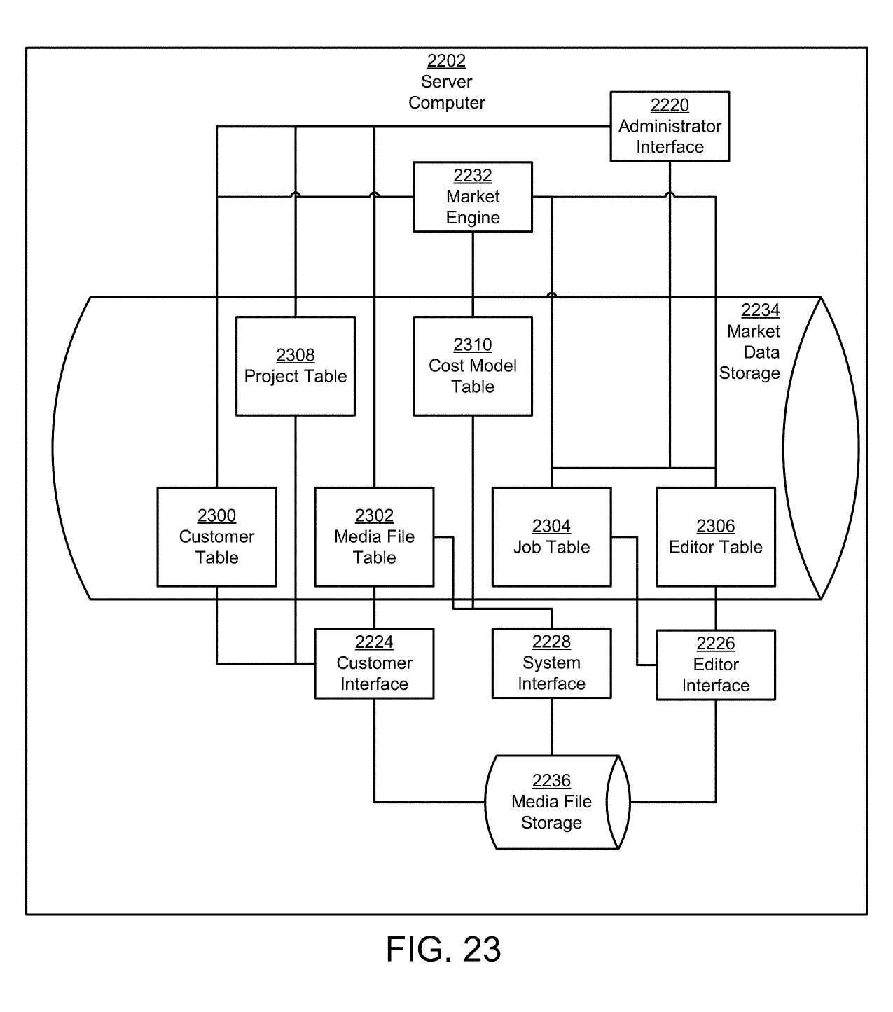

According to various embodiments illustrated by FIG. 22, the market engine 2232 is configured to both add jobs to the transcription job market provided by the transcription system 2200 and to maintain the efficiency of the transcription job market once the market is operational. To achieve these goals, in some embodiments, the market engine 2232 exchanges market information with the customer interface 2224, the administrator interface 2230, the editor interface 2226, the system interface 2228, the transcript database system interface 2240, the market data storage 2234, and the media file storage 2236. Market information may include any information used to maintain the transcription job market or stored within the market data storage 2234. Specific examples of market information include media file information, job information, customer information, editor information, administrator information and transcription request information. Each of these types of information is described further below with reference to FIG. 23.

In some embodiments, the transcript database system interface 2240 is configured to exchange information with the transcript database system 2238 via an application program interface (API) exposed by the transcript database system 2238. The transcript database system interface 2240 can thereby transmit information, such as EHR entries documenting a patient encounter, to the transcript database system 2238 and/or receive information, such as transcripts documenting previous patent encounters within the EHR, from the transcript database system 2238. The EHR entries transmitted via the transcript database system interface 2240 may include audio entries transcribed by the processes executed by the transcription system 2200 and stored in the market data storage 2234 as draft or final transcription information. Examples of EHR systems that the transcript database system interface 2240 is configured to exchange information with include EHR systems provided by AthenaHealth, Epic Systems, Allscripts, eClinicalWorks, and Cerner. More generally, at least some embodiments of the transcript database system interface 204 can exchange information with any text storage database, such as a MySQL database, an Oracle database, a MongoDB database, or a Redis Database, or any web application connected to a text storage database.

In some embodiments, the market engine 2232 is configured to identify unprocessed media files stored in the media file storage 2236. In some of these embodiments, the market engine 2232 identifies unprocessed media files after receiving an indication of the storage of one or more unprocessed media files from another component, such as the customer interface 2224, which is described further below. In others of these embodiments, the market engine 2232 identifies unprocessed media files by periodically executing a query, or some other identification process, that identifies new, unprocessed media files by referencing information stored in the market data storage 2234 or the media file storage 2236. In some embodiments, the market engine 2232 is also configured to send a request for ASR processing of unprocessed media files to the system interface 2228. This request may include information specifying that only a limited portion of the unprocessed media file (e.g., a specified time period) be processed. Further, in at least one embodiment, the market engine 2232 tracks completion percentage of the draft transcription during subsequent ASR processing. The market engine 2232 may store, in the market data storage 2234, the completion percentage associated with partial transcriptions stored in the media file storage 2236.

In these embodiments, the system interface 2228 is configured to receive requests for ASR processing, and, in response to these requests, provide the unprocessed media files to the ASR device 2222, along with any requested limits on the ASR processing. The ASR device 2222 is configured to receive a media file, to perform transcoding and automatic speech recognition on the received media file in accord with the request and to respond with draft transcription information that includes a draft (synchronized or non-synchronized) transcription of the content of the received media file and a predicted cost of editing the draft transcription. This predicted cost, referred to herein as the ASR_cost is based on information computed as part of the ASR processing and a cost model. The cost model may be a general model or may be associated with the project, customer or editor associated with the media file. A project is a set of media files grouped by a customer according to domain, due date and time or other media file attribute. Projects are described further below. Cost models predict the cost of editing a draft transcription and are described further with reference to FIG. 23 below. The system interface 2228 is further configured to receive the draft transcription information, store the draft transcription information in the media file storage 2236, store the location of the draft transcription information in the market data storage 2234, and notify the market engine 2232 of the availability of the draft transcription information.

In one example illustrated by FIG. 22, the market engine 2232 receives an identifier of a newly stored media file from the customer interface 2224. Responsive to receipt of this identifier, the market engine 2232 provides a request to perform ASR processing on the media file to the system interface 2228. The system interface 2228, in turn, retrieves the media file from the media file storage 2236 and provides the media file, along with a set of parameters that indicate appropriate language, acoustic, cost and formatting models, to the ASR device 2222. The ASR device 2222 responds with draft transcription information that includes a synchronized draft transcription, lattices, search statistics, ASR_cost and other associated data. The system interface 2228 receives the draft transcription information, stores the draft transcription information in the media file storage 2236, stores the location of the draft transcription information in the market data storage 2234 and notifies the market engine 2232 of the availability of the draft transcription information.

In other embodiments, the market engine 2232 is configured to perform a variety of processes in response to receiving a notification that draft transcription information is available. For instance, in one example, the market engine 2232 employs natural language processing techniques to determine the type of content or domain included in the media file associated with the draft transcription information and stores this information in the market data storage 2234. In another example, the market engine 2232 determines the duration of the content included in the media file and stores the duration in the market data storage 2234. In another example, after receiving a notification that draft transcription information is available, the market engine 2232 determines an initial pay rate for editing the draft transcription included in the draft transcription information and stores job information associated with the draft transcription in the market data storage 2234. In this example, the initial pay rate included in the job information is determined using the due date and time, difficulty, duration, domain and ASR_cost of the media file associated with the draft transcription information. In other examples, other combinations of these factors may be used, or these factors may be weighted differently from one another. For instance, in one example, due date and time and duration may be replaced with times-real-time. In another example, the weight applied to any particular factor may be 0.

In other embodiments, the market engine 2232 is configured to periodically publish, or “push,” notifications to editors that indicate the availability of new jobs. In one of these embodiments, the market engine 2232 tailors these notifications by sending them only to particular editors or groups of editors, such as those editors who have permission to edit the jobs. In other embodiments, the market engine 2232 tailors notifications based on other job characteristics, such as the type of job (editing, QA, etc), difficult, domain or due date and time. In some examples, the market engine 2232 sends notifications to editors based on their ability to complete jobs having the attribute to which that the notification is tailored. Continuing the previous examples, the market engine 2232 may send notifications to editors who may assume particular roles (editor, QA, etc.), who have a track record of handling difficult jobs, who are well versed in a particular domain, or who are highly efficient.

In at least one embodiment, the market engine 2232 notifies editors of near-term future job availability based on the upstream workflow. In this embodiment, as files are uploaded by customers and processed by the ASR device, the market engine 2232 predicts how many more jobs will be available and based on one or more the attributes of these jobs, such as duration, domain, etc., the market engine 2232 sends out advanced notice to one or more editors via the editor interface 2226.

In other embodiments, the market engine 2232 is configured to determine the difficulty of successfully editing the draft transcription and to store the difficulty in the market data storage 2234. In these embodiments, the market engine 2232 may base this determination on a variety of factors. For example, in one embodiment, the market engine 2232 calculates the difficulty using an equation that includes weighted variables for one or more of the following factors: the content type (domain) of the media file, the historical difficulty of media files from the customer (or the project), the draft transcription information, and acoustic factors (such as noise-level, signal-to-noise-ratio, bandwidth, and distortion).

In some embodiments, the market engine 2232 is configured to create and post jobs corresponding to unedited media files, thereby making the jobs available to the editors for claiming and completion. According to one example, as part of this processing, the market engine 2232 stores an association between each job and a media file targeted for work by the job. This action is performed so that factors affecting pay rate, such as those described above, can be located in a media file table.

As described further below with reference to the editor interface 2226, editors claim jobs by indicating their preferences on a user interface provided by the editor interface 2226. After a job is claimed, the job is removed from the market, so that no other editors can access the job. However, until the editor has actually begun to edit the job, it is relatively easy for the job to be put back on the market. Typically, leaving the original claim in place is preferred. However, in some embodiments, the market engine 2232 is configured to determine whether the editor who claimed the job will be able to complete the job before the due date and time. In these embodiments, the market engine 2232 is configured to make this determination based on the job characteristics (difficulty, domain, duration, etc.) and the editor's historical proficiency as stored in the market data storage 2234. For example, the editor may be associated with a times-real-time statistic stored in the market data storage 2234. The times-real-time statistic measures editor productivity and is calculated by dividing the time it takes for the editor to complete each job by the duration of the media file associated with each job. In some embodiments, the market engine 2232 is configured to use this statistic to estimate the completion time of the job (based on duration multiplied by times-real-time). In some embodiments, the market engine 2232 is configured to condition this statistic based on job attributes, and thus compute the statistic from similar jobs performed by the editor in the past. The set of historical jobs used to compute the times-real-time statistic may include all jobs performed by the editor, a subset of jobs which have similar attributes to the present job, or other combinations of historical jobs, including those that were not performed by the editor. The market engine 2232 may calculate this statistic as a mean, a median, a duration-weighted mean, or using summaries of historical processing times for the editor or other editors for different media file subsets.

In other embodiments, if the market engine 2232 determines that an editor may be unlikely to complete a job before the due date and time, the market engine 2232 may reverse the assignment and put the job back on the market, thus allowing some number of other editors to claim the job. In some these embodiments, the market engine 2232 determines the likelihood that the editor will complete the job before its due date and time using one or more of the following factors: historical productivity of the editor (in general or, more specifically, when editing media files having a characteristic in common with the media file associated with the job); the number of jobs currently claimed by the editor; the number of jobs the editor has in progress; and the due dates and times of the jobs claimed by the editor. When the market engine 2232 reverses an assignment, the original editor is informed of this condition via the editor interface 2226. The market engine 2232 may or may not allow the original editor to reclaim the job from the market, depending on whether data indicates interest of other editors in the job. One example of an indicator of interest is whether the job is being previewed by any other editors. Another factor which may influence this decision is if the total volume of unedited draft transcriptions exceeds a threshold.

In some embodiments, the market engine 2232 determines a likelihood of completion for each possible combination of editor and job. In these embodiments, the market engine 2232 may calculate this likelihood using any combination of the factors discussed above (historical productivity, number of jobs claimed, number of jobs in progress, due dates and times of claimed jobs, etc.). Further, in some embodiments, the market engine 2232 prevents editors from claiming jobs for which the editor's likelihood of completion metric transgresses a threshold. In these embodiments, the threshold is a configurable parameter. Further, according to these embodiments, the market engine 2232 may prevent an editor from claiming a job in a variety of ways including rejecting an offer from the editor to claim the job and causing the job to not be display to the editor within the editor interface 2226 via, for example, a meta rule. Meta rules are discussed further below.

In other embodiments, if the market engine 2232 determines that an editor may be unlikely to complete a job before the due date and time, the market engine 2232 sends a notification to the editor who claimed the job via the editor interface 2226. The notification may include a variety of information, such as a notification that the job may be revoked shortly or including a link to allow the editor to voluntarily release the job.

In several embodiments, the market engine 2232 is configured to give permission to many editors to edit the same draft transcription and to offer all editors the same pay rate to do so. In some alternative embodiments, however, the market engine 2232 is configured to determine if, based on historical information, some editors display an increased proficiency with particular types of media files (for example in certain domains) and to increase the pay rate for these editors when transcribing media files having the particular type. In addition, some embodiments of the market engine 2232 are configured to adjust the pay rate based on overall editor experience levels, as well as the historical productivity of the editors, both in general and on the type of media file for which the rate is being set.

In general, the market engine 2232 sets the pay rate based on the aforementioned factors, such as job difficulty, required times-real-time, and ASR_cost. However, to maintain an efficient market in some embodiments, the market engine 2232 is configured to determine when market conditions suggest intervening actions and to, in some cases, automatically take those intervening actions. For example, when the market is saturated with non-difficult jobs, an abnormally large amount of unassigned, difficult jobs may develop. According to this example, to correct the inefficiency in the market, the market engine 2232 intervenes by increasing the pay rate of difficult jobs or decreasing the pay rate of low difficulty jobs. In still another example, the market engine 2232 intervenes to increase the pay rate of a job where the proximity of the current date and time and due date and time for the media file associated with the job transgresses a threshold.

In some embodiments, the market engine 2232 is configured to use the preview functionality as an indicator of job difficulty and appropriate pay rate. For instance, in one example, the market engine 2232 detects that the number of editors who have previewed a job and not claimed it has exceeded a threshold. Alternatively, in another example, the market engine 2232 detects that the total preview duration of an unclaimed job has transgressed a threshold. These phenomena may indicate that the job is more difficult than is reflected by the current pay rate. The market engine 2232 may then intervene to increase the pay rate to improve the chance that the job will be claimed or to split the media file into segments.

Additionally, in some embodiments, the market engine 2232 monitors the status of, and information associated with, all jobs available on the market. This information includes difficulty, pay rate, due date and time, domain and summary information such as the number of editors with permission to edit a draft transcription, the amount of time a job has been on the market, the number of previews of the media file associated with a job, and other data concerning the market status of the job and its associated media file. In some embodiments, the market engine 2232 is configured to use this information to ensure that problem jobs are accepted. For example, the market engine 2232 may increase the pay rate, may enable a larger number of editors to access to the file, or may cut the file into shorter segments—thus producing several less difficult editing jobs for the same media file.

In other embodiments, the market engine 2232 is configured to, under certain conditions, hide some of the low difficulty jobs in order to create a more competitive environment or to induce editors to work on difficult jobs. Additionally, in some embodiments, the market engine 2232 is configured to encourage the editors to accept less desirable jobs by bundling jobs together with more desirable jobs. For example, the market engine 2232 may group a selection of jobs with variable difficulty together so that a single editor would need to claim all of these jobs, instead of claiming only low difficulty jobs. Other characteristics that may determine the desirability of a job, and which may be used to determine the bundling, include customer, project, domain (e.g. interesting content), and historical time waiting on the market for the customer/project.

In some embodiments, the market engine 2232 is configured to analyze the overall status of the market prior to modifying job characteristics. For instance, in one example, the market engine 2232 monitors the amount of work available in the market, and if the amount transgresses a threshold, increases the pay rate for jobs that are within a threshold value of their due dates and times. In other embodiments, the market engine 2232 is configured to analyze the dynamics of the overall market to determine intervening actions to perform. In one example, the market engine 2232 measures the rate at which jobs are being accepted and measures the number of jobs or duration of the jobs, and estimates the time at which only the least popular jobs will remain in the market. If the market engine 2232 determines that this time is sufficiently ahead of the due date and time for these jobs, then the market engine 2232 may wait before increasing the pay rate.

In other embodiments, the market engine 2232 is configured to set meta rules to affect the behavior of the market. Meta rules globally modify the behavior of the market by affecting how all or some of the available jobs will appear on the market. For instance, the market engine 2232 may set a meta rule that prevents some percentage of the jobs from being available to any editors for a certain time period. The market engine 2232 may use this rule during periods when there is a surplus of work, and therefore help to smooth out the flow of files through the system. Or, the market engine 2232 may set a meta rule to make files available only to relatively inexperienced editors for a certain time period. The market engine 2232 may use this rule where many relatively easy jobs are being processed by the market, so that the market presents a good opportunity to give less experienced editors more work in learning how to efficiently operate the editing platform. Or, the market engine 2232 may set a meta rule that automatically send some percentage of jobs to multiple editors for cross-validation. Various embodiments may implement a variety of meta rules, and embodiments are not limited to a particular meta rule or set of meta rules.

In other embodiments, the market engine 2232 is configured to implement a rewards program to encourage editors to claim difficult jobs. In one embodiment, the market engine 2232 issues rewards points to editors for completing files and bonus points for completing difficult files. In this embodiment, the editor interface 2226 is configured to serve a rewards screen via the user interface rendered on the client computer 2206. The rewards screen is configured to receive requests to redeem reward and bonus points for goods and services or access to low difficulty media files.

In some embodiments, the market engine 2232 is configured to estimate the expected completion time of the editing job and further refine the market clearing processes discussed above. If the market engine 2232 determines that the current progress is not sufficient to complete the file on time, the editor may be notified of this fact via the editor interface 2226, and, should the condition persist, the market engine 2232 is configured to make the job available to other editors (i.e. to put the jobs back on the market). In some circumstances, the market engine 2232 may revoke the entire job from the original editor. In this case, the job is put back on the market as if no work had been done. In other cases, the market engine 2232 may dynamically split the job at the point where the original editor has completed editing, creating one or more new jobs that are comprised of the remaining file content. The market engine 2232 puts these one or more new jobs on the market, and the original editor is paid only for the completed work.

In some embodiments, the market engine 2232 is configured to process a delivery request or partial delivery request received from another component, such as the customer interface 2224. In response to receiving a partial delivery request targeting a media file being processed in a job, the market engine 2232 dynamically splits the job at the point where the original editor has completed editing and creates one or more new jobs that are comprised of the remaining file content. The market engine 2232 puts these one or more new jobs on the market, and the original editor is paid only for the completed work. It is appreciated that the splitting functionality described herein may apply to any jobs being processed by the transcription system 2200, such as QA jobs. In another embodiment, in response to receiving a partial delivery request targeting a media file being processed in a job, the market engine 2232 stores one or more segments of the transcription up to the point where the editor has completed editing without interrupting the job.

In other embodiments, the market engine 2232 is configured to perform a variety of processes after receiving an indication that a job has been completed. For example, if a newly completed draft transcription information was split into segments, then the market engine 2232 concatenates completed segments together into a completed transcript. Conversely, where the job was directed to transcription of audio entries describing a patient encounter for the EHR, the market engine 2232 may either preserve segments for each section of the EHR or divide the completed transcript into segments for each distinct EHR section. Regardless, in examples directed to EHR transcripts, the market engine 2232 may transmit one or more segments and/or whole transcripts to the transcript database system 2238 via the transcript database system interface 2240 upon completion of a job.

In another example, the market engine 2232 is configured to compare a completed synchronized transcript with the draft transcription produced by the ASR device 2222. In this example, the market engine 2232 uses the number of corrections performed on the transcript to compute a standard distance metric, such as the Levenshtein distance. The market engine 2232 stores this measurement in the market data storage 2234 for later use in determining an objective difficulty for the editing job.

In various embodiments, the market engine 2232 is configured to use the objective difficulty in a variety of processes. For example, in some embodiments, the market engine 2232 uses the objective difficulty for a set of jobs to adjust the historical times-real-time statistic for an editor to determine the actual price that the customer pays for the transcription service, or as input to the automated difficulty-determination process discussed herein.

In other embodiments, the market engine 2232 is configured to, prior to making the completed transcript available to the customer, create and post a new job to validate the completed transcription or the completed segments of a transcription. For example, in one embodiment, the market engine 2232 creates and posts a QA job on the same market as the editing jobs. This QA job may target completed transcriptions or a completed segment of a transcription. A subset of editors may be qualified for the QA role, and the profiles of this subset may include a QA attribute. These editors would then be permitted to view, preview, and claim the QA jobs in the market via the editor interface 2226. However, in some examples, the editor of the original transcript would not have permission to QA their own job, even if the editor in general is qualified to perform in a QA role. The profiles of some editors may include a QA attribute, but lack an editor attribute. These editors would only be permitted to view, preview, and claim QA jobs.

As the QA jobs normally require much less work than the original editing job, in some embodiments, the market engine 2232 is configured to set the pay rate for the QA jobs at a lower level. However, in other embodiments, the market engine 2232 is configured to monitor and adjust the pay rate for the QA jobs as for the editing jobs, with similar factors determining the pay rate, including file difficulty, the ASR_cost, the proximity of the due date and time, and the media file duration. Additionally, in some embodiments, the market engine 2232 is configured to use QA-specific factors to determine the pay rate for QA jobs. For example, in one embodiment, the market engine 2232 adjusts the pay rate based on the number of flags in the edited transcript, the historical proficiency of the original editor, the times-real-time it took to produce the completed transcription, and the ASR distance metric for the media file. Flags are set during the editing process and indicate problem content within the edited transcript. For example, flags may indicate content that is unclear or that requires additional research to ensure accurate spelling. In some embodiments, the flags are standardized to facilitate automatic processing by the components of the transcription system.

After this QA processing is complete, in some embodiments, the market engine 2232 is configured to make the final synchronized transcription or its final synchronized segments available to the customer, who may then download the transcription or transcription segments for his or her own use via the customer interface 2224. Additionally or alternatively, the market engine 2232 may transmit one or more segments and/or whole final transcriptions to the transcript database system 2238 via the transcript database system interface 2240.

In some embodiments, to periodically measure editor proficiency, the market engine 2232 is configured to allow a media file to be edited by multiple editors. For instance, in one example, the market engine 2232 periodically creates several different editing jobs from the same media file, and these jobs are claimed and processed by multiple editors. The market engine 2232 tracks the underlying media file and does not assign more than one of these jobs to the same editor. After several editors edit the same file, the market engine 2232 executes a ROVER or similar process to determine intra-editor agreement, and thereby assign quality scores to individual editors, the quality score being proportional to the number of words in the editor's final transcript, which have high agreement among the other editors. In addition, the market engine 2232 may use the ROVER process to produce the final transcript. In this case, the market engine 2232 may assign different weights to different editors based on the editor characteristics (domain or customer expertise, historical transcription proficiency, etc).

In other embodiments, the market engine 2232 is configured to build cost models that are used to determine predicted costs for editing draft transcriptions. In some of these embodiments, the market engine 2232 is configured to generate cost models based on variety of information including historical productivity information, such as times-real-time statistics and ASR distance information. Further, in these embodiments, the cost models may be specific to particular editors, customers or projects. For instance, in one example, the market engine 2232 builds cost models that accept a unique identifier for a media file, the ASR information (synchronized draft transcription, lattices, search statistics, acoustic characteristics) for the media file, and an indication of an editor, customer or project associated with the media file and that return a projected transcription cost that is conditioned on historical productivity associated with the editor, customer or project. Once these models are built, the market engine 2232 stores them in the media file storage 2236.

In some embodiments, customers may be given access to the transcripts for final editing via the customer interface 2224. In these embodiments, the market engine 2232 uses the customer edits as the gold-standard reference for computing editor accuracy. In other embodiments, the market engine 2232 is configured to use times-real-time, stored in the market data storage at the time of j ob upload, as a factor in determining editor proficiency. Typically, the market engine 2232 also adjusts the editing time (and thus the historical editing productivity for editors) by an objective difficulty, such as the ASR distance, because more difficult files will necessarily take longer to edit.

As described above, in some examples, customers are given access to edit transcription and caption information associated with synchronized derived content (e.g., clips or clip reels). FIG. 12 illustrates one example screen 1200 served by the customer interface 124 that supports this function. As shown in FIG. 12, the screen 1200 includes transcription information section 1202 and video clip captioning results section 1204. The transcription information section 1202 highlights text that is associated with synchronized derived content. The transcription information section 1202 further includes an edit word button, a delete word button, and an edit paragraph button that facilitate editing of the transcription information. In response to receiving input selecting any of these buttons, the screen 1200 provides one or more user interface elements or executes other processes that perform the function recited in the name of the button. The video clip captioning results section 1204 includes a graphical representation of the locations within the media file where portions of the clip may be found.

In some embodiments, the customer interface 2224 is configured to provide a user interface to the customer 2210 via the network 2216 and the client computer 2204. For instance, in one embodiment, the customer interface 2224 is configured to serve a browser-based user interface to the customer 2210 that is rendered by a web-browser running on the client computer 2204. In another embodiment, the mobile recording application 118 acts as the user interface (or a portion thereof) and interoperates with the customer interface 2224 via its transcription system interface 206. Regardless, in these embodiments, the customer interface 2224 exchanges customer and media file information with the customer 2210 via the user interface.

Media file information may include one or more media files, information associated with the one or more media files, or information descriptive of the attributes of the one or more media files. Specific examples of media file information include a media file to be transcribed, content derived from the media file (e.g., captions and caption placement information), a type of content included in a media file, a date and time a transcription of a media file is due, a domain of the subject matter presented in the content, a unique identifier of a media file, storage location of a media file, subtitles associated with a media file, annotations associated with a media file, semantic tagging associated with a media file, and advertising associated with a media file. Media file information is described further below with reference to FIG. 23. According to an example illustrated by FIG. 22, the customer interface 2224 receives media file information from the user interface. This media file information includes a media file, information indicating a date and time that transcription of the media file is due, and a type of content included in the media file. Responsive to receipt of this media file information, the customer interface 2224 stores the media file in the media file storage 2236 and stores a unique identifier of the media file, the due date and time, and the content type in the market data storage 2234.

According to an example illustrated by FIG. 22, the customer interface 2224 receives media file information from the user interface. This media file information includes a media file and media file information indicating a domain of the subject matter of the content included in the media file or a project to be associated with the media file from which the domain may be derived. Responsive to receipt of this media file information, the customer interface 2224 stores the media files in the media file storage 2236 and stores a unique identifier of the media file and other media file information in the market data storage 2234.

According to another example illustrated by FIG. 22, the customer interface 2224 provides media file information to the user interface. This media file information includes unique identifiers of one or more media files previously received from the customer 2210, the due dates and times associated with the received media files, and the project information associated with the received media files. In this example, the customer interface 2224 receives modifications to the provided media file information made by the customer 2210 via the user interface. Responsive to receiving the modifications, the customer interface 2224 stores the modifications in the market data storage 2234.

According to another example illustrated by FIG. 22, the customer interface 2224 provides media file information to the user interface. This media file information includes one or more unique identifiers of one or more media files previously received from the customer 2210 and other attributes of these files including, for example, the due dates and times, content types, prices, difficulties, and statuses or states of jobs associated with the previously received media files. As discussed above with reference to FIG. 33, examples of job states include New, ASR_In_Progress, Available, Assigned, Editing_In_Progress, and Complete. In some embodiments, the customer interface 2224 serves media file information as one web page, while in other embodiments, the customer interface 2224 serves this media file information as multiple web pages. It is to be appreciated that different due dates and times and content type may be associated with different prices to the customer. Customer prices may also be impacted by other factors that impact the underlying transcription cost, including how objectively difficult the media file transcription is to edit, as described above.

In another example, the customer interface 2224 serves media file information that includes final transcription information to the user interface rendered by the client computer 2204. This final transcription information includes a final (synchronized or non-synchronized) transcription of the content included in a media file. The synchronized transcription is comprised of a textual representation of the content of the media file, where each textual token has associated with it indicia of the location in the media file to which it applies. The textual tokens may include words, numerics, punctuation, speaker identification, formatting directives, non-verbal indicators (such as [BACKGROUND NOISE], [MUSIC], [LAUGHTER], [PAUSING]) and other markings that may be useful in describing the media file content. The empty string may also be used as a textual token, in which case the location indicia serves to keep the transcription synchronized with the media file content in the absence of useful textual information. In the case of the draft transcription from the ASR device, these empty-string tokens may be used if the ASR process was confident that some transcription-worthy event has occurred at that location, but is unsure of the particular identity of that event. In this case, having the location indicia associated with the event facilitates synchronized correction by the editor.

In other embodiments, the customer interface 2224 is configured to receive a request to edit final transcription information from the user interface, and in response to the request, to provide an editing platform, such as the editing screen described below with reference to the editor interface 2226, to the user interface. In this example, the editing platform enables customers to edit the final transcription information. Also, in this example, user interface includes elements that enable the customer 2224 to initiate an upload of the edited final transcription information to the customer interface 2224. The customer interface 2224, in turn, receives the edited final transcription information, stores the final transcription information in the media file storage 2236 and stores an association between the edited final transcription information and the media file with content that was transcribed in the market data storage 2234.

In other embodiments, the customer interface 2224 is configured to provide screens within the user interface to exchange voice macro configuration information with a user. These screens may be used to setup and edit voice macros that can be processed by a voice macro processor (e.g. the voice macro processor 210) resident on the server computer 2202, either of the client computers 2204 or 2212, or the mobile computing device 100. In some embodiments, voice macro configuration information maintained via these screens is stored in the market data storage 2234 and transmitted to any of the various devices described above when changes are made to ensure that each voice macro processor has a current configuration. For example, in some embodiments, the customer interface 2224 is configured to exchange voice macro configuration information with the mobile recording application 118 via the transcription system interface 206.

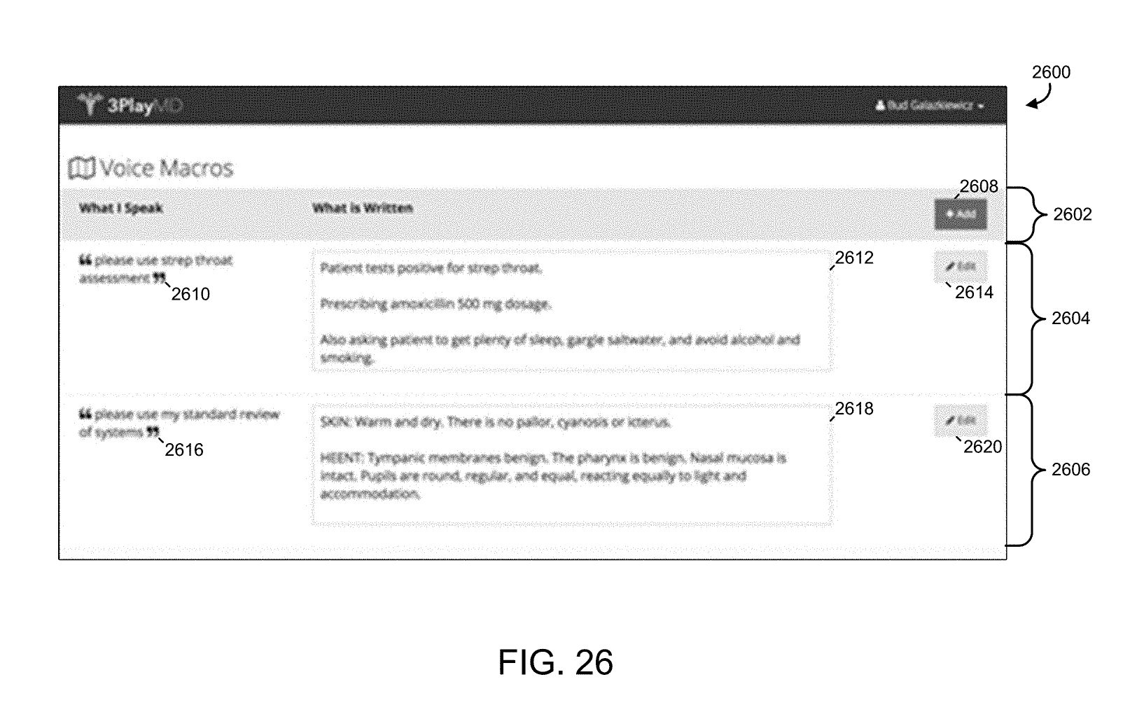

FIG. 26 illustrates one example of such a voice macro screen 2600. As shown in FIG. 26, the voice macro screen 2600 includes an add voice macro control 2602 and edit voice macro controls 2604 and 2606. The add voice macro control 2602 includes an add control 2608 and text descriptive of the purpose of voice macros. The edit voice macro control 2604 includes textbox controls 2610 and 2612 and edit control 2614. The edit voice macro control 2606 includes textbox controls 2616 and 2618 and edit control 2620.

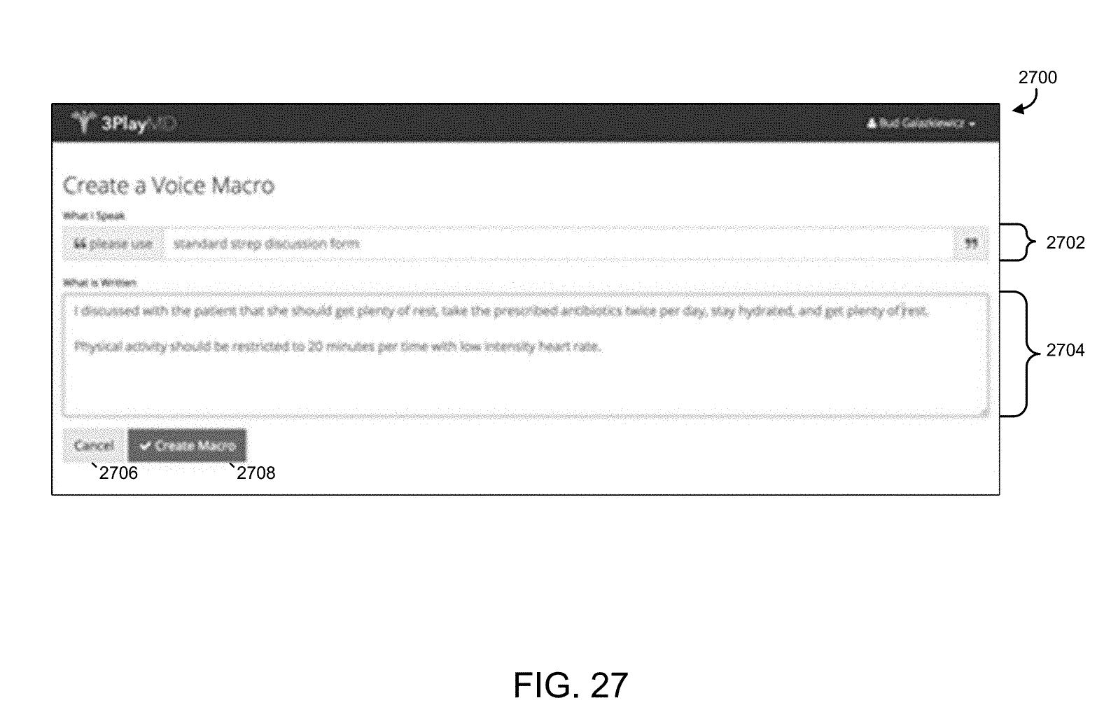

When presenting the voice macro screen 2600, the user interface is configured to receive selections of elements of the voice macro screen 2600. Where the user interface receives input selecting the add control 2608, or either of the edit controls 2614 or 2620, the user interface presents a voice macro edit screen (e.g., the voice macro edit screen 2700 described further below with reference to FIG. 27).

As shown in FIG. 27, the voice macro edit screen 2700 includes voice macro trigger control 2702, voice macro expansion text control 2704, cancel control 2706, and create voice macro control 2708. The content presented in the voice macro trigger control 2702 and the voice macro expansion text control 2704 varies depending on whether the user interface displays the voice macro edit screen 2700 in response to a selection of an add control or an edit control. More specifically, where an add control was selected, the voice macro edit screen includes no content in the voice macro trigger control 2702 and the voice macro expansion text control 2704. However, where an edit control was selected, in the voice macro trigger control 2702 and the voice macro expansion text control 2704 include the content of the textbox controls of the edit control selected.

When presenting the voice macro screen 2700, the user interface is configured to process input directed to elements of the voice macro screen 2700. For instance, where the user interface receives input directed to the voice macro trigger control 2702, the user interface adjusts the text presented therein to match the input. Similarly, where the user interface receives input directed to the voice macro expansion text control 2704, the user adjusts text presented therein to match the input. Where the user interface receives a selection of the create voice macro control 2708, the user interface stores the contents of the voice macro trigger control 2702 and the voice macro expansion text control 2704 within a data structure configured to store voice macros. Such voice macro data structures may be stored, for example, in the market data storage 2234. Stored voice macros may be used to replace trigger text with expansion text as described herein.

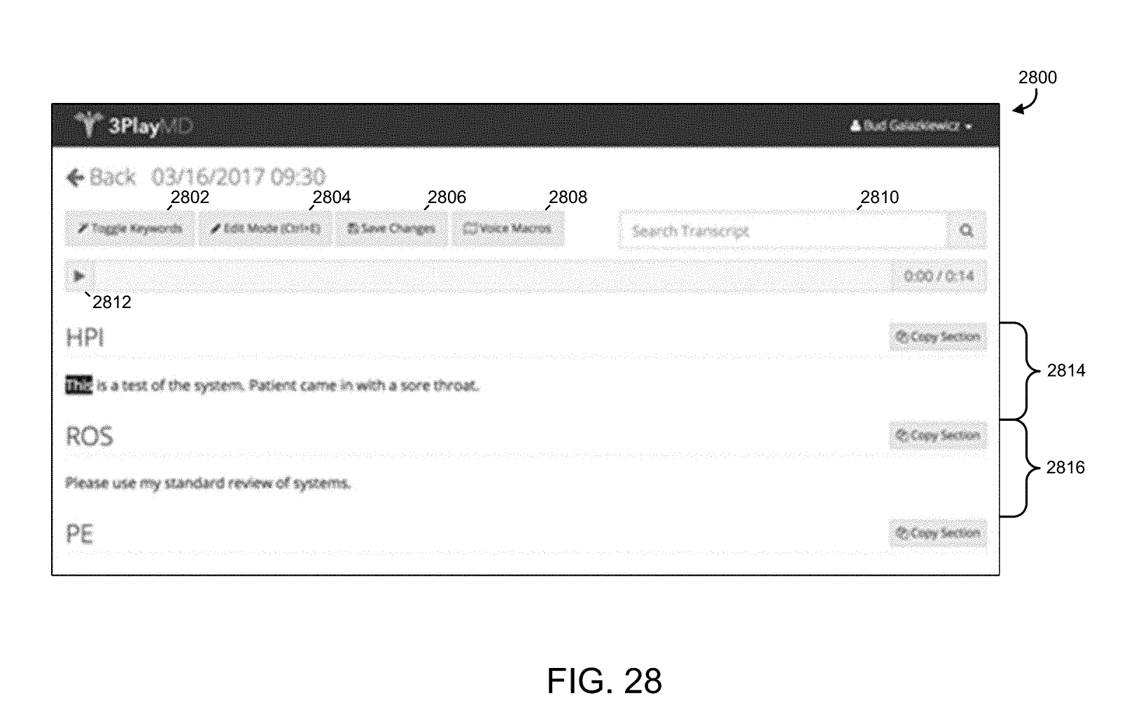

In other embodiments, the customer interface 2224 is configured to provide screens within the user interface to preview and edit transcripts. FIG. 28 illustrates one example of such an edit screen 2800. As shown in FIG. 28, the edit screen 2800 includes a toggle keywords control 2802, an edit mode control 2804, a save control 2806, a voice macros control 2808, a search transcript control 2810, a transcript playback control 2812, section controls 2814 and 2816. Each of the section controls 2814 and 2816 correspond to an EHR section and present ASR-generated transcript text of audio entries for each section. As shown in FIG. 28, each of the section controls 2814 and 2816 includes a copy section control.

When presenting the edit screen 2800, the user interface is configured to process input directed to elements of the edit screen 2800. For instance, where the user interface receives input selecting the toggle keywords control 2802, the user interface either highlights, or removes highlighting from, a list of keywords found within the transcript text presented by the section controls 2814 and 2816. As shown in FIG. 28, “This” is a highlighted keyword. In some examples, the list of keywords is a configurable parameter stored in the market data store 2234.

Where the user interface receives input selecting the edit mode control 2804, the user interface enables modification to the transcript text presented in the section controls 2814 and 2816. Where the user interface receives input selecting the save control 2806, the user interface stores the transcript text as currently presented in the section controls 2814 and 2816. Where the user interface receives input selecting the voice macros control 2808, the user interface presents a voice macro screen (e.g., the voice macro screen 2600 describe above with reference to FIG. 26). Where the user interface receives input selecting the search transcript control 2810, the user interface receives text defining a search string and/or executes a search using the search string. Results of the search are presented in the section controls 2814 and 2816. Where the user interface receives input selecting the transcript playback control 2812, the user interface renders audio entries that transcribed into the transcript text presented in the section controls 2814 and 2816. Where the user interface receives input selecting the copy section control of either of the section controls 2814 and 2816, the user interface copies the transcript text presented in the section control to a clipboard.

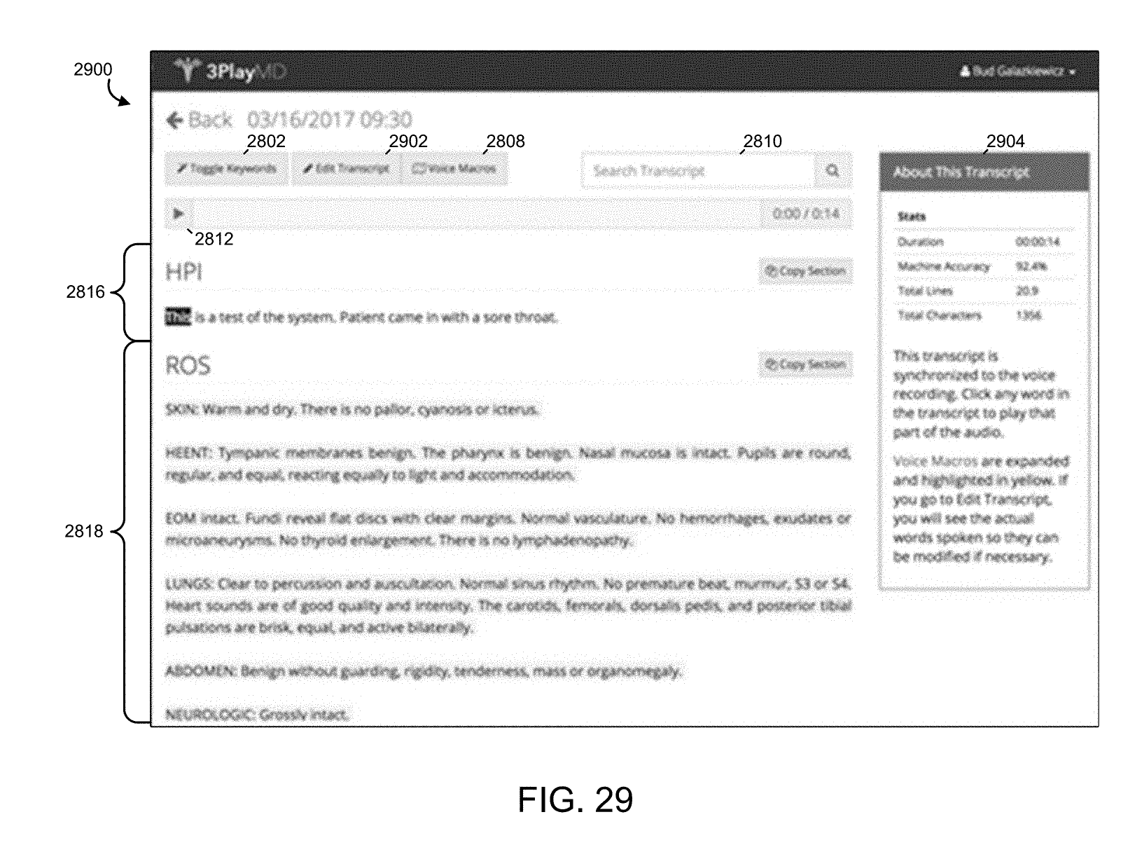

FIG. 29 illustrates an example of the preview screen 2900. As shown, the preview screen 2900 includes several of the elements of the edit screen 2800 (e.g., the toggle words control 2802, the voice macros control 2808, the search transcript control 2810, the transcript playback control 2812, and the section controls 2816 and 2818). These elements of the preview screen 2900 are structured and function similarly to the elements of the edit screen 2800. As shown, the preview screen 2900 also includes an edit transcript control 2902 and a summary control 2904. The summary control 2904 provides a variety to statistics regarding the transcript being displayed. These statistics may include the duration of the audio entries transcribed to render the transcript text presented in the section controls 2816 and 2818, the accuracy of the ASR processing, the total number of lines in the transcript, and the total number of characters in the transcript.

When presenting the preview screen 2900, the user interface is configured to process input directed to elements of the preview screen 2900. For instance, where the user interface receives input selecting the edit transcript control 2902, the user interface presents an edit screen (e.g., the edit screen 2800 described above with reference to FIG. 28). In addition, when presenting the preview screen 2900, the user interface is configured to implement any configured voice macros by replacing trigger text within the section controls 2816 and 2818 with expansion text. FIG. 29 illustrates as example of this feature within the section control 2818. As shown in FIG. 29, the trigger text “Please use my standard review of systems.” from FIG. 28 has been replaced with the text highlighted within FIG. 29.

Although the examples described above focus on a web-based implementation of the customer interface 2224, embodiments are not limited to a web-based design. Other technologies, such as technologies employing a specialized, non-browser-based client, may be used to implement the user interface without departing from the scope of the aspects and embodiments disclosed herein. For instance, according to one embodiment, the customer interface 2224 is a simple, locally executed upload client that allows the customer to do nothing more than upload media files to the server via FTP or some other protocol. In other embodiments, the customer interface 2224 is configured to perform a variety of processes in response to exchanging information via the user interface. For instance, in one embodiment, after receiving one or more media files via the user interface, the customer interface 2224 provides the market engine 2232 with an identifier of newly stored, unprocessed media files.

In some embodiments, the customer interface 2224 is configured to provide a system interface to the client computer 2204 via the network 2216. For instance, in one embodiment, the customer interface 2224 implements an HTTP API through which the client computer 2204 exchanges transcription request information with the customer interface 2224. The transcription request information may include request type information (e.g., an identifier indicating that the transcription request information includes an automatic synchronization request), project information (e.g., an identifier of a project), customer information (e.g. an identifier of a customer), media file information (e.g., an identifier of a media file or derived content), boolean values used to synchronize reference content with derived content, values of one or more thresholds used to synchronize reference content with derived content, identifiers of one or more requested transcription products, a delivery point identifier, and responses to any requests. In some embodiments, the delivery point identifier may include URI's, URL's, an FTP folder identifier (along with authentication credentials), or the like. In response to receiving the transcription request information, the customer interface 2224 may store the transcription request information in the market data storage 2234 in association with the identifier of the media file, project, or customer for which the requested transcription products are to be generated. In addition, responsive to receiving the transcription request information, the customer interface 2224 may store the media file identified in the transcription request information in the media file storage 2236. Transcription request information is described further below with reference to FIG. 23.

In some embodiments, the customer interface 2224 is configured to perform a variety of processes in response to exchanging information via the system interface with the client computer 2204. For instance, in one embodiment, after receiving transcription request information specifying a request for partial delivery of one or more transcription products, the customer interface 2224 provides the request for delivery (or partial delivery) to the market engine 2232.

In some embodiments, the administrator interface 2230 is configured to provide a user interface to the administrator 2214 via the network 2220 and the client computer 2208. For instance, in one embodiment, the administrator interface 2230 is configured to serve a browser-based user interface to the administrator 2214 that is rendered by a web-browser running on the client computer 2208. In this embodiment, the administrator interface 2230 exchanges market information with the administrator 2214 via this user interface. Market information may include any information used to maintain the transcription job market and stored within the market data storage 2234. Specific examples of market information include a media file information, job information, customer information, editor information, administrator information and transcription request information. Market information is described further below with reference to FIG. 23. Using the administrator interface 2230, the administrator 2214 acts as a transcription manager who regulates the transcription job market as a whole to promote its efficient allocation of resources.

In these embodiments, the administrator interface 2230 is also configured to receive a request from the user interface to provide a preview of a media file, and in response to the request, serve a preview screen for the requested media file to the user interface. This preview screen provides the content of the media file and the draft transcription associated with the media file. More particular, in some embodiments, the preview screen is configured to provide the media file content, in the form of, for example, a streamed version of the original file, as well as the draft transcription information for the media file, which includes time-codes or frame-codes. This information enables the preview screen to display the draft transcription in synchronization with the media file content. A preview may consist of all or some of this information.

According to an example illustrated by FIG. 22, the administrator interface 2230 provides media file information to the user interface. This media file information includes one or more unique identifiers of one or more media files previously received from the customer 2210, the content types associated with the received media files and the difficulties associated with the received media files. In this example, responsive to receipt of an indication that the administrator 2214 wishes to preview a media file, the administrator interface 2230 provides a preview of the media file and the draft transcription information associated with the media file. Further, in this example, the administrator interface 2230 receives modifications to the provided media file information made by the administrator 2214 via the user interface. Responsive to receiving the modifications, the administrator interface 2230 stores the modifications in the market data storage 2234.

In other embodiments, the administrator interface 2230 is also configured to receive a request from the user interface to provide an administrator view of all jobs available on the market, and in response to the request, serve an administrator screen to the user interface. This administrator view is configured to display the same information available to editors viewing the job market (difficulty, pay-rate, due date and time, domain, etc.), and also displays additional information to assist the administrator. For example, the administrator view may display the number of editors with permission to edit each available media file, the amount of time each job has been on the market, the number of previews of the media file, and other data concerning the market status of the media file. In this way, the administrator view displays information that enables administrators to ensure that the media file is accepted as an editing job.

The administrator interface 2230 is also configured receive a request from the user interface to modify information displayed by administrator view, and in response to the request, store the modified information. Thus, the administrator view may increase the pay rate, may manually enable a larger number (or smaller number) of editors access to the file, or may cut the file into shorter segments—thus producing several editing jobs for the same media file. The administrator view may also bundle jobs together to ensure that all editors have access to a reasonable cross-section of work. For example, the administrator view may group a selection of jobs with variable difficulty together so that a single editor would need to accept all of these jobs, instead of just picking low difficulty jobs for themselves. The administrator view may also throttle the supply of low difficulty jobs in order to create a more competitive environment or to induce editors to work on difficult jobs. The administrator view may also record as accepted a claim offer that is higher than the pay rate for a job.

In other embodiments, the administrator interface 2230 is also configured to receive a request from the user interface to provide a meta rules view, and in response to the request, serve a meta rules screen to the user interface. Meta rules globally modify the behavior of the market by affecting how all or some of the available jobs will appear on the market. In some embodiments, the administrator interface 2230 is configured receive a request from the user interface to add to or modify meta rules displayed by meta rules view, and in response to the request, store the newly introduced meta rule information.

In other embodiments, the administrator interface 2230 is also configured to receive a request from the user interface to provide a market view of jobs available on the market, and in response to the request, serve a market screen to the user interface. The market screen is configured to provide summarized information about jobs organized according to one or more job (or associated media file) attributes. For instance, one example of the market screen displays all of the jobs assigned to one or more editors. In another example, the market screen displays all jobs organized by due date and time in the form of a calendar. In yet another example, the market screen displays all jobs belonging to a particular customer.

Although the examples described above focus on a web-based implementation of the administrator interface 2230, embodiments are not limited to a web-based design. Other technologies, such as technologies employing a specialized, non-browser-based client, may be used without departing from the scope of the aspects and embodiments disclosed herein.

In some embodiments, the editor interface 2226 is configured to provide a user interface to the editor 2212 via the network 2218 and the client computer 2206. For instance, in one embodiment, the editor interface 2226 is configured to serve a browser-based user interface to the editor 2212 that is rendered by a web-browser running on the client computer 2206. In this embodiment, the editor interface 2226 exchanges media file information, editor information and job information with the editor 2212 via this user interface. Editor information may include information associated with an editor profile or the history of an editor within the transcription job market. Job information may include information associated with transcription jobs that are available or that have been completed via the transcription job market. Specific examples of editor information include a unique identifier of the editor, domains of subject matter in which the editor is qualified to work, and identifiers of currently claimed jobs. Specific examples of job information include a unique identifier of the job, a deadline for the job, and a pay rate for the job. Media file information, editor information and job information are described further below with reference to FIG. 23.

In these embodiments, the editor interface 2226 is configured to provide job information only for jobs that the editor 2212 is permitted to work. In one example, the editor interface 2226 determines that an editor is permitted to edit a draft transcription based on a complex of factors. If a media file associated with the draft transcription has a specific content type, then in some examples, the editor interface 2226 will only provide job information associated with the media file to editors qualified to edit that specific content type. In other examples, the editor interface 2226 may provide job information associated with more difficult files to more experienced editors. In still other examples, the editor interface 2226 provides job information for jobs associated with specific customers to particular subset of editors. This approach may be advantageous, for example, if there are confidentiality concerns and only that subset of editors have signed non-disclosure agreements. Thus, examples of the editor interface 2226 do not provide job information to the editor 2212 for jobs claimed by another editor or for jobs that the editor 2212 does not have permission to claim.

In other embodiments, the editor interface 2226 is configured to receive a request from the user interface to provide a preview of a media file, and in response to the request, serve a preview screen for the requested media file to the user interface. This preview screen provides the content of the media file and the draft transcription information associated with the media file. Editors may be given access to the preview screen for a media file before they choose to accept the editing job at the given pay rate. The preview screen includes the media file content, in the form of, for example, a streamed version of the original media file, as well as the draft transcription information for the media file, which includes time-codes or frame-codes. This information enables the preview screen to display and draft transcription in synchronization with playback of the media file content. A preview may consist of all or some of this content. The editors may access the preview screen content and thereby assess for themselves the difficulty of the editing job, and then make a judgment as to whether they are willing to accept the job at the current pay rate. This enables editors to select content that they are interested in and to reveal their expertise or preferences for subject matter that would otherwise by unknown to administrators. In aggregate this will tend to improve transcription quality since the jobs will be better matched to editors than if randomly assigned.

According to an example illustrated by FIG. 22, the editor interface 2226 provides job information to the user interface. This job information includes one or more unique identifiers of one or more jobs available for the editor 2212, identifiers of the media files associated with the jobs, pay rates of the jobs, domain information, and durations of the content of the media file associated with the job. In this example, responsive to receipt of an indication that the editor 2212 wishes to preview a media file, the editor interface 2226 provides a preview of the media file and the draft transcription information associated with the media file. If the editor 2212 wishes to claim the job, the editor 2212 indicates this intent by interacting with the user interface and the user interface transmits a request to claim the job for the editor 2212 to the editor interface 2226. Next, in this example, the editor interface 2226 receives the request to claim an available job from the user interface, and responsive to receiving this request, the editor interface 2226 records the job as claimed in the market data storage 2234.

In other embodiments, the editor interface 2226 is configured to receive a request from the user interface to edit a draft transcription, and in response to the request, serve an editing screen to the user interface. The editing screen is configured to provide a variety of tools for editing and correcting the draft transcription. For instance, the editing screen provides access to the original file (or a converted version of the original file) along with the draft transcription information by referencing information contained in both the market data storage 2234 and the media file storage 2236. For instance, in at least one embodiment, the editing screen includes a side panel that indicates whether there is any metadata associated with particular portions of transcript text.

In some embodiments directed to editing EHR draft transcriptions, the editing screen is configured to indicate which EHR sections are to be reviewed (e.g., by graying out unselected sections) and/or restrict review only to selected EHR sections by displaying only the selected sections. As described above with reference to FIG. 33, the selected sections may be specified by JSON objects included in the transcription request information for the job. In some embodiments, only a subset of nearby, but unselected, sections of the EHR are displayed in conjunction with selected sections to provide useful context while minimizing screen usage. In any of these embodiments, all or a portion of the audio entries for the selected and unselected sections may be provided to the editor or quality assurance user context.

In other embodiments directed to editing EHR draft transcriptions, the editing screen includes an expand macros control configured to replace, within the editing screen, trigger text with expansion text. In these embodiments, the editing screen is configured to interoperate with a voice macro processor (e.g., the voice macro processor 210) resident on the server computer 2202. This feature enables editors to modify expansion text in accordance with user instructions. For example, in these embodiments, if the draft transcription recites “Please use my standard review of systems template, but add slight abdomen tenderness,” the editing screen initially displays transcript text as recognized by ASR processing. The editor may then click the expand macros control, which will expand the text according to the stored voice macro record. The editor may then amend the transcript text which recites “Abdomen: Normal” to recite “Abdomen: Slightly tender to touch.” Next, the editor can delete the remaining “but add slight abdomen tenderness” from the transcript text. A voice macro can also be used to record this additional “exception” voice macro both for present (in the current transcript review) and future use (e.g. in future audio entries) by the user. Additionally, it is appreciated that the editing screen may be used by the editor to correct trigger text that was not properly translated by ASR processing. After correcting the trigger text, the editor may generate expansion text for further editing by selecting the expand macros control.

In one embodiment, once an editor begins working on a job, the editing screen provides the complete media file content and synchronized draft transcription information for editing using client-computer-based editing software. The editor interface 2226 also transitions the job into a working state by recording the working state for the job in the market data storage 2234.

The editing process consists of playing the media file content, and following along with the draft transcription, modifying the draft transcription information as necessary to ensure that the saved draft transcription reflects the content of the media file. According to some embodiments, as the editor modifies the draft transcription information, the editing screen communicates with the editor interface 2226 to indicate progress through the editing job. The editing screen tracks the time point into the file that the editor is playing, as well as the parts of the draft transcription information that has been modified in order to estimate progress. The progress is communicated back to the editor interface 2226, and the editor interface 2226 then stores this progress in the market data storage 2234 in association with the editing job. In the course of editing a job, the editor may come across words and phrases that are difficult to understand. The editing screen allows editors to flag these regions, so that they may be reviewed and possibly corrected by an administrator. A flag may indicate complete unintelligibility or may include a guess as to the correct word, but with an indicator that it is a guess. For each job, the prevalence of corrected flags in the edited transcript is stored in the market data storage 2234, and the market engine 2232 may use stored flags as an indicator of editor proficiency to aid with future job assignment. In some embodiments, the editing screen allows editors to store auxiliary deliverables such as search keywords, descriptive summarization, and other metadata derived from the transcription information during editing jobs and QA jobs.

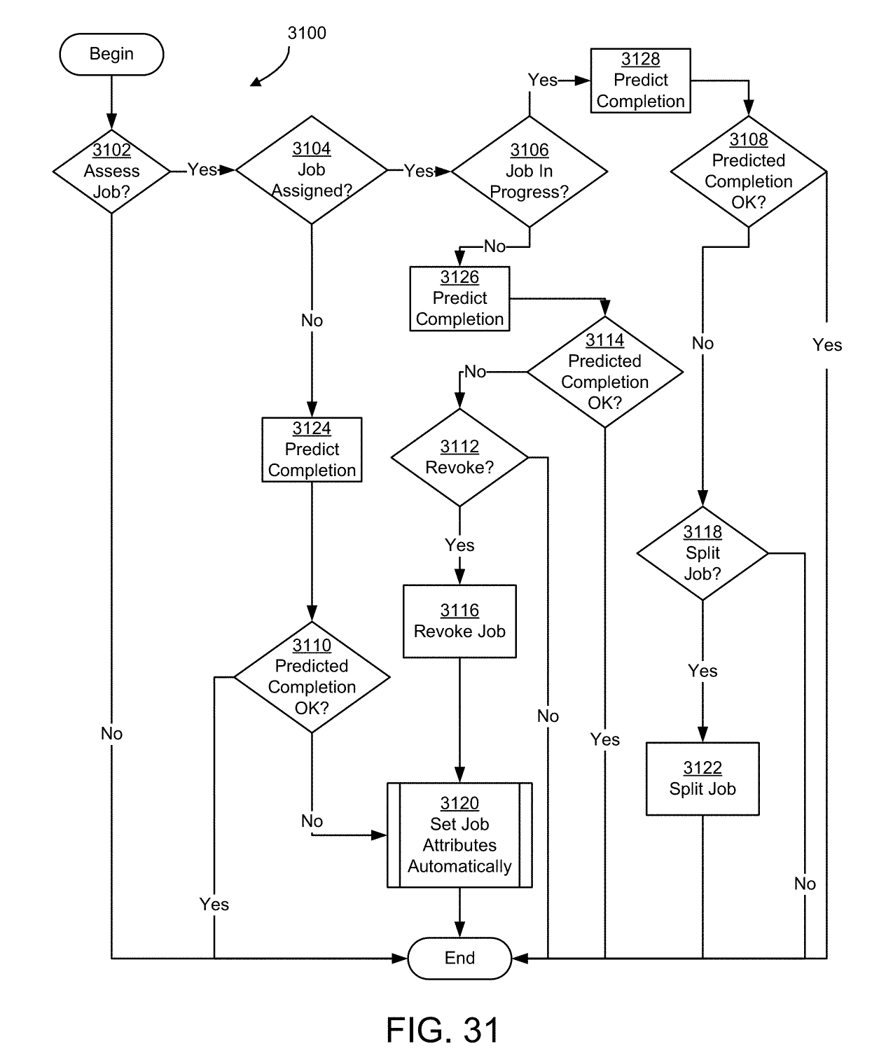

In other embodiments, the editor interface 2226 is configured to receive a request from the user interface to save an edited draft transcription, and in response to the request, save the edited draft transcription to the media file storage 2236 and update progress information for the job in the market data storage 2234. In some embodiments, saving the progress information triggers estimation of a new completion date and time, which is then evaluated relative to the due date and time as discussed with reference to FIG. 31 below.

According to an example illustrated by FIG. 22, the editor interface 2226 provides job information to the user interface. This job information includes one or more unique identifiers of one or more jobs available for the editor 2212, identifiers of the media files associated with the jobs, pay rates of the jobs, durations of the content of the media file associated with the job and progress the editor 2212 has made editing the draft transcription associated with the job. In this example, responsive to receipt of an indication that the editor 2212 wishes to edit the draft transcription, the editor interface 2226 serves an editing screen to the user interface.

In some embodiments, the editing screen is configured to receive an indication that the editor has completed a job. In these embodiments, the editing screen is also configured to, in response to receiving the indication, store the edited draft transcription information as final transcription information in the media file storage 2236 and update the market data storage 2234 to include an association between the media file and the final transcription information.

The examples described above focus on a web-based implementation of the editor interface 2226. However, embodiments are not limited to a web-based design. Other technologies, such as technologies employing a specialized, non-browser-based client, may be used without departing from the scope of the aspects and embodiments disclosed herein.

Each of the interfaces disclosed herein may both restrict input to a predefined set of values and validate any information entered prior to using the information or providing the information to other components. Additionally, each of the interfaces disclosed herein may validate the identity of an external entity prior to, or during, interaction with the external entity. These functions may prevent the introduction of erroneous data into the transcription system 2200 or unauthorized access to the transcription system 2200.

FIG. 23 illustrates the server computer 2202 of FIG. 22 in greater detail. As shown in FIG. 23, the server computer 2202 includes the market engine 2232, the market data storage 2234, the customer interface 2224, the system interface 2228, the editor interface 2226, and the media file storage 2236. In the embodiment illustrated in FIG. 23, the market data storage 2234 includes a customer table 2300, a media file table 2302, a job table 2304, an editor table 2306, a project table 2308 and a cost model table 2310.

In the embodiment of FIG. 23, the customer table 2300 stores information descriptive of the customers who employ the transcription job market to have their media files transcribed. In at least one embodiment, each row of the customer table 2300 stores information for a customer and includes an customer_id field, and a customer_name field. The customer_id field stores an identifier of the customer that is unique within the transcription job market. The customer_name field stores information that represents the customer's name within the transcription job market. The customer_id is used as a key by a variety of functions disclosed herein to identify information belonging to a particular customer.

The media file table 2302 stores information descriptive of the media files (e.g., reference files and derived content files) that have been uploaded to the transcription job market for transcription. In at least one embodiment, each row of the media file table 2302 stores information for one media file and includes the following fields: media_file_id, customer_id, state, duration, due_date_and_time, difficulty, domain, ASR_cost, proposed_pay_rate, ASR_transcript_location, edited_transcript_location, QA_transcript_location, advertisement, transcript_product1, transcript_product2, etc. . . . . The media_file_id field stores a unique identifier of the media. The customer_id field stores a unique identifier of the customer who provided the media file. The state field stores information that represents the state of the media file. The duration field stores information that represents the duration of the content of the media file. The due_date_and_time field stores information that represents the date and time by which the customer requires a transcription be complete. The difficulty field stores information that represents an assessed difficulty of completing a transcription of the media file. The domain field stores information that identifies a subject matter domain to which the media file belongs. The ASR_cost field stores information that represents a predicted cost of transcribing the media file as assessed using draft transcription information. The proposed_pay_rate field stores information that represents a pay rate proposed using draft transcription information. The ASR_transcript_location field stores an identifier of a location of draft transcript information associated with the media file. The edited_transcript_location field stores an identifier of a location of edited draft transcript information associated with the media file. The QA_transcript_location field stores an identifier of a location of QA transcription information associated with the media file. The advertisement field stores one or more identifiers of one or more locations of one or more advertisements associated with the media file. The transcript_product1, transcript_product2, etc. . . . store identifiers of locations of other transcription products or other derived content associated with the media file (e.g., products that may be uploaded via the customer interface 2224 or generated by the transcription system 2200). The media_file_id is used as a key by a variety of functions disclosed herein to identify information associated with a particular media file.

The job table 2304 stores information descriptive of the jobs to be completed within the transcription job market. In at least one embodiment, each row of the job table 2304 stores information for one job and includes the following fields: job_id, media_file_id, deadline, state, job_type, pay_rate, editor_id, progress, flags, XRT, corrections, hide, ASR_distance. The job_id field stores an identifier of the job that is unique within the transcription job market. The media_file_id field stores the unique identifier of the media file to be transcribed by an editor working the job. The deadline field stores information that represents the date and time by which the job must be complete. The state field store the current state (or status) of the job. Examples values for the state field include New, ASR_In_Progress, Available, Assigned, Editing_In_Progress, and Complete. The job_type field stores information that represents a type of work that must be performed to complete the job, for example editing, QA, etc. The pay_rate field stores information that represents a pay rate for completing the job. The editor_id field stores the unique identifier of the editor who has claimed this job. The progress field stores information that represents an amount of work completed for the job. The flags field stores information that represents the number and type of flags assigned to the job during editing, as described above. The XRT field stores information that represents the times-real-time statistic applicable to the job. The corrections field stores information that represents corrections made to the draft transcription as part of the job. The hide field stores information that determines whether components, such as the market engine 2232 and the editor interface 2226, should filter out the job from job views. The ASR_distance field stores information that represents the number of changes from the draft transcription made as part of the job. The job_id is used as a key by a variety of functions disclosed herein to identify information associated with a particular job.

The editors table 2306 stores information descriptive of the editors who prepare transcriptions within the transcription job market. In at least one embodiment, each row of the editors table 2306 stores information for one editor and includes the following fields: editor_id, roles, reward_points, domains, and special_capabilities. The editor_id field stores an identifier of the editor that is unique within the transcription job market. The roles field stores information representative of roles that the editor is able to assume with the transcription job market, for example, editor, QA, etc. Examples of these roles include editor and QA editor. The reward_points field stores information that represent the number of reward points accumulated by the editor. The domains field stores information that represents subject matter domains of media files that the editor has permission to edit. The special_capabilities field stores information that represents specialized skills that the editor possesses. The editor_id is used as a key by a variety of functions disclosed herein to identify information belonging to a particular editor.

In the embodiment of FIG. 23, the project table 2308 stores information descriptive of projects that the transcription job market is being utilized to complete. In at least one embodiment, each row of the project table 2308 stores information for a project and includes an project_id field, a project_name field, a customer_id field, and a domain field. The project_id field stores information that identifies a group of media files that belong to a project. The project_name field stores information that represents the project's name within the transcription job market. The customer_id field indicates the customer to whom the project belongs. The domain field stores information that identifies a subject matter domain of media files included in the project. The project_id is used as a key by a variety of functions disclosed herein to identify information grouped into a particular project.

In the embodiment of FIG. 23, the cost model table 2310 stores information descriptive of one or more cost models used to predict the cost of editing the content included media files. In at least one embodiment, each row of the cost model table 2310 stores information representative of a cost model and includes an editor_id field, a customer_id field, a project_id field and a Cost_Model_Location field. The editor_id field stores the unique identifier of an editor to whom the cost model applies. The customer_id field stores the unique identifier of a customer to whom the cost model applies. The project_id field stores the unique identifier of a project to which the cost model applies. The Cost_Model_Location field stores information identifying a location of the cost model. The editor_id, customer_id or project_id, any of which may be null or the wildcard indicator, may be used as a key by a variety of functions disclosed herein to identify a location of a cost model applicable to any of these entities.

The transcription request table 2312 stores information descriptive of requests for delivery of transcription products. In at least one embodiment, each row of the transcription request table 2312 stores information for one transcription request and includes the following fields: media_file_id, project_id, customer_id, delivery_point, transcription_product, and quality_thresholds. The media_file_id field stores a unique identifier of a media file that is the basis for the requested transcription products. The customer_id field stores a unique identifier of the customer who provided the transcription request. The delivery_point field stores an identifier of a location to which the requested transcription products may be transmitted. The transcription_product field stores identifiers of the requested transcription products, which include derived content such as transcriptions, captions, caption positioning information, and the like. The quality_thresholds field stores values of one or more quality thresholds associated with one or more potential delivery types. The delivery types may be defined by points in time, transcription status, or derived content status.

Various embodiments implement the components illustrated in FIG. 23 using a variety of specialized functions. For instance, according to some embodiments, the customer interface 2224 uses a File_Upload function and a File_Update function. The File_Upload function uploads a file stored on a customer's computer to the server computer 2202 and accepts parameters including customer_id, project_id, filename, and optionally, domain. The customer_id parameter identifies the customer's unique customer_id. The project_id identifies the project to which the media file belongs. The filename parameter specifies the name of the media file or derived content file to be uploaded by the customer interface 2224. The domain parameter specifies the subject matter domain to which the media file belongs. In at least one embodiment, if the domain parameter is not specified, the market engine 2232 determines the value of the domain parameter from the value of the domain field of a record stored within the project table 2308 that has a project_id field that is equal to the project_id parameter.

In other embodiments, the File_Update function updates an attribute of a media file record and accepts parameters including media_file_id, attribute, and value. The media_file_id parameter identifies the media file record with attributes that will be modified as a result of execution of the File_Update function. The attribute parameter identifies an attribute to be modified. In at least one embodiment, this attribute may be the domain, difficulty or state of the media file, as stored in the media file table 2302. The value parameter specifies the value to which the attribute is to be set as a result of executing the File_Update function.

In other embodiments, the system interface 2228 uses a File_Send_to_ASR function and a File_Create_Draft function. The File_Send_to_ASR function provides a media file to the ASR device 2222 and causes the ASR device 2222 to perform automatic speech recognition on the content included in the media file. The File_Send_to_ASR function accepts parameters including media_file_id. The media_file_id parameter identifies the media file to be processed by the ASR device 2222.

In other embodiments, the File_Create_Draft function creates draft transcription information for a media file and accepts parameters including media_file_id and ASR_output. The media_file_id parameter identifies the media file for which the draft transcription information will be created by execution of the File_Create_Draft function. The ASR_output parameter specifies the location of the ASR output generated by the ASR device 2222 during its processing of the media file.

In other embodiments, the market engine 2232 uses the following functions: File_Assess_Difficulty, File_Propose_Pay_Rate, File_Compute_Actual_Difficulty, Job_Create, Job_Split, Job_Adjust_Parameter and Job_Revoke. The File_Assess_Difficulty function determines an estimated difficulty to transcribe the content included in a media file and accepts parameters including a media_file_id. The media_file_id parameter identifies the media file including the content for which difficulty is being accessed.

In other embodiments, the File_Propose_Pay_Rate function determines an initial pay rate for transcribing the content included in a media file and accepts parameters including media_file_id and draft_transcription_information. The media_file_id parameter identifies the media file for which the proposed_pay_rate that will be determined as a result of execution of the File_Propose_Pay_Rate function. The draft_transcription_information parameter specifies the location of the draft_transcription_information associated with the media file. The File_Propose_Pay_Rate function determines the initial pay_rate using the information included in the draft_transcription_information.

In other embodiments, the File_Compute_Actual_Difficulty function determines an actual difficulty of transcribing the content included in a media file and accepts parameters including media_file_id (from which it determines the location of the draft_transcription_information and final_transcription_information from the media file table 2302. The media_file_id parameter identifies the media file for which the actual difficulty will be determined as a result of execution of the File_Compute_Actual_Difficulty function. The File_Compute_Actual_Difficulty function determines the actual difficulty by comparing the content of the draft transcription included in the draft transcription information to the content of the final transcription included in the final transcription information. In one embodiment, File_Compute_Actual_Difficulty function uses the number of corrections performed on the transcription to compute a standard distance metric, such as the Levenshtein distance. The File_Compute_Actual_Difficulty function stores this measurement in the ASR_distance field of the job table 2304.

In other embodiments, the Job_Create function creates a job record and stores the job record in the job table 2304. The Job_Create function and accepts parameters including media_file_id, job_type, pay_rate and, optionally, deadline. The media_file_id parameter identifies the media file for which the job is being created. The job_type parameter specifies the type of editing work to be performed by an editor claiming the job. The pay_rate parameter specifies the amount of pay an editor completing the job will earn. The deadline parameter specifies the due date and time for completing the job.

In other embodiments, the Job_Split function segments a job into multiple jobs and accepts parameters including job_id and a list of timestamps. The job_id parameter identifies the job to be segmented into multiple jobs. The list of timestamps indicates the location in the media file at which to segment the media file to create new jobs.

In other embodiments, the Job_Adjust_Attribute function modifies the value of an attribute stored in a job record and accepts parameters including job_id, attribute and value. The job_id parameter identifies the job record with an attribute to be modified. The attribute parameter identifies an attribute to be modified. In at least one embodiment, this attribute may be the pay_rate, deadline, XRT, or ASR_distance of the job record, as stored in the job table 2304. The value parameter specifies the value to which the attribute is to be set as a result of executing the Job_Adjust_Attribute function.

In other embodiments, the Job_Revoke function removes a job from an editor and makes the job available for other editors to claim according to the current market rules. The Job_Revoke function accepts parameters including job_id. The job_id parameter identifies the job to be revoked.

In other embodiments, the Deliver_Product function transmits one or more transcription products to a delivery point via the customer interface 2224 and accepts parameters including a product_id, and delivery_point. The product_id parameter identifies the transcription product to be delivered to the location identified by the delivery_point parameter.

In other embodiments, the editor interface 2226 uses the following functions: Job_Store_Output, Job_Update_Progress, Job_List_Available, Job_Preview, Job_Claim, and Job_Begin. The Job_Store_Output function stores the current version of the edited draft transcription and accepts parameters including a job_id. The job_id parameter identifies the job for which the current version of the edited draft transcription is being stored.

In other embodiments, the Job_Update_Progress function updates the progress attribute included in a job record and saves the current state of the transcription. The Job_Update_Progress function accepts parameters including job_id, transcription data and progress. The job_id parameter identifies the job record for which the progress attribute will be updated to the value specified by the progress parameter. The transcription data is saved to the location specified in the media file record associated with the job_id.

In other embodiments, the Job_List_Available function returns a list of jobs available to an editor and accepts parameters including editor_id, and optionally, job_type, domain, difficulty, deadline, and proposed_pay_rate. The editor_id parameter identifies the editor for which the list of available jobs is being created. The job_type parameter specifies a job_type to which each job in the list of available jobs must belong. The domain parameter specifies a domain to which each job in the list of available jobs must belong. The difficulty parameter specifies a difficulty that the media file associated with the job in the list must have. The deadline parameter specifies a deadline that each job in the list of available jobs must have. The proposed_pay_rate parameter specifies a proposed_pay_rate that the media file associated with the job must have. It is to be appreciated that meta rules, may also impact the list of jobs returned by the Job_List_Available function.

In other embodiments, the Job_Preview function causes a preview screen to be provided to a user interface and accepts parameters including editor_id and job_id. The editor_id parameter identifies the editor for which the preview is being provided. The job_id parameter specifies the job that is being previewed.

In other embodiments, the Job_Claim function records a job as claimed and accepts parameters including editor_id and job_id. The editor_id parameter identifies the editor for which the job is being claimed. The job_id parameter specifies the job that is being claimed.

In other embodiments, the Job_Begin function causes an editing screen to be provided to a user interface and accepts parameters including job_id. The job_id parameter specifies the job associated with the draft transcription to be edited.

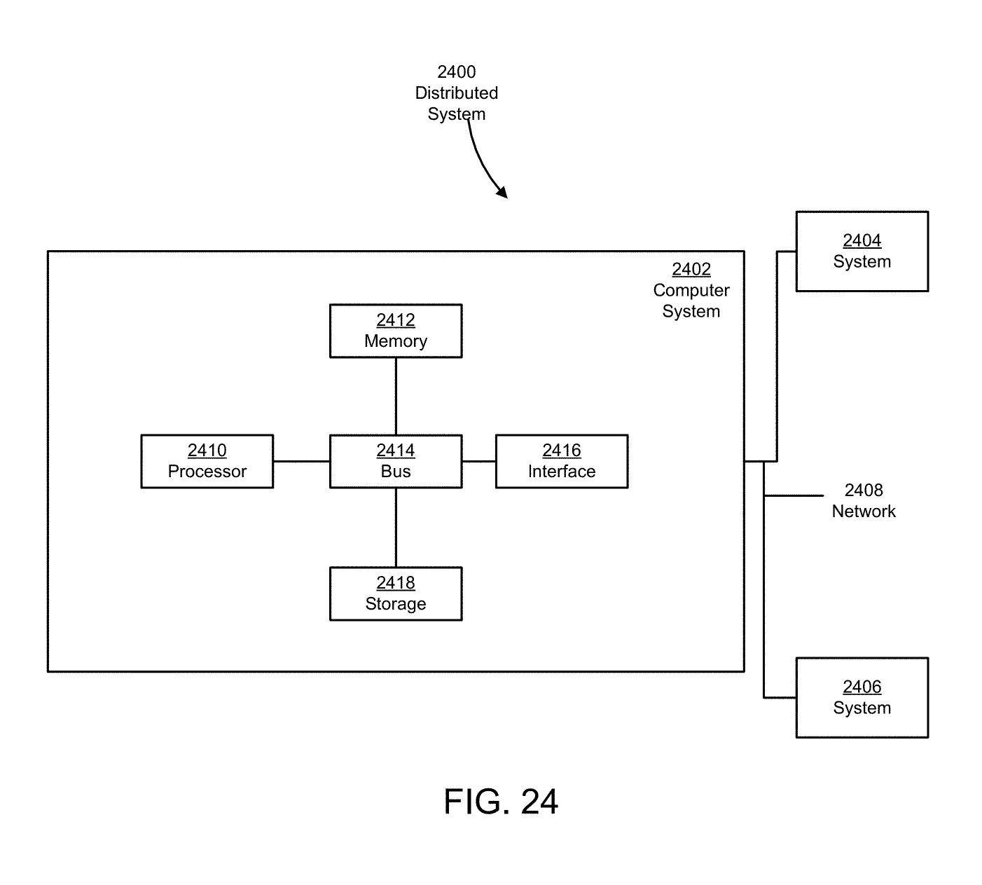

Embodiments of the transcription system 2200 are not limited to the particular configuration illustrated in FIGS. 22 and 23. Various examples utilize a variety of hardware components, software components and combinations of hardware and software components configured to perform the processes and functions described herein. In some examples, the transcription system 2200 is implemented using a distributed computer system, such as the distributed computer system described further below with regard to FIG. 24.

### Computer System

As discussed above with regard to FIG. 22, various aspects and functions described herein may be implemented as specialized hardware or software components executing in one or more computer systems. There are many examples of computer systems that are currently in use. These examples include, among others, network appliances, personal computers, workstations, mainframes, networked clients, servers, media servers, application servers, database servers and web servers. Other examples of computer systems may include mobile computing devices, such as cellular phones and personal digital assistants, and network equipment, such as load balancers, routers and switches. Further, aspects may be located on a single computer system or may be distributed among a plurality of computer systems connected to one or more communications networks.

For example, various aspects and functions may be distributed among one or more computer systems configured to provide a service to one or more client computers, or to perform an overall task as part of a distributed system. Additionally, aspects may be performed on a client-server or multi-tier system that includes components distributed among one or more server systems that perform various functions. Consequently, examples are not limited to executing on any particular system or group of systems. Further, aspects and functions may be implemented in software, hardware or firmware, or any combination thereof. Thus, aspects and functions may be implemented within methods, acts, systems, system elements and components using a variety of hardware and software configurations, and examples are not limited to any particular distributed architecture, network, or communication protocol.

Referring to FIG. 24, there is illustrated a block diagram of a distributed computer system 2400, in which various aspects and functions are practiced. As shown, the distributed computer system 2400 includes one more computer systems that exchange information. More specifically, the distributed computer system 2400 includes computer systems 2402, 2404 and 2406. As shown, the computer systems 2402, 2404 and 2406 are interconnected by, and may exchange data through, a communication network 2408. The network 2408 may include any communication network through which computer systems may exchange data. To exchange data using the network 2408, the computer systems 2402, 2404 and 2406 and the network 2408 may use various methods, protocols and standards, including, among others, Fibre Channel, Token Ring, Ethernet, Wireless Ethernet, Bluetooth, IP, IPV6, TCP/IP, UDP, DTN, HTTP, FTP, SNMP, SMS, MMS, SS7, JSON, SOAP, CORBA, REST and Web Services. To ensure data transfer is secure, the computer systems 2402, 2404 and 2406 may transmit data via the network 2408 using a variety of security measures including, for example, TLS, SSL or VPN. While the distributed computer system 2400 illustrates three networked computer systems, the distributed computer system 2400 is not so limited and may include any number of computer systems and computing devices, networked using any medium and communication protocol.

As illustrated in FIG. 24, the computer system 2402 includes a processor 2410, a memory 2412, a bus 2414, an interface 2416 and data storage 2418. To implement at least some of the aspects, functions and processes disclosed herein, the processor 2410 performs a series of instructions that result in manipulated data. The processor 2410 may be any type of processor, multiprocessor or controller. Some exemplary processors include commercially available processors such as an Intel Xeon, Itanium, Core, Celeron, or Pentium processor, an AMD Opteron processor, a Sun UltraSPARC or IBM Power5+ processor and an IBM mainframe chip. The processor 2410 is connected to other system components, including one or more memory devices 2412, by the bus 2414.

The memory 2412 stores programs and data during operation of the computer system 2402. Thus, the memory 2412 may be a relatively high performance, volatile, random access memory such as a dynamic random access memory (DRAM) or static memory (SRAM). However, the memory 2412 may include any device for storing data, such as a disk drive or other non-volatile storage device. Various examples may organize the memory 2412 into particularized and, in some cases, unique structures to perform the functions disclosed herein. These data structures may be sized and organized to store values for particular data and types of data.

Components of the computer system 2402 are coupled by an interconnection element such as the bus 2414. The bus 2414 may include one or more physical busses, for example, busses between components that are integrated within a same machine, but may include any communication coupling between system elements including specialized or standard computing bus technologies such as IDE, SCSI, PCI and InfiniBand. The bus 2414 enables communications, such as data and instructions, to be exchanged between system components of the computer system 2402.

The computer system 2402 also includes one or more interface devices 2416 such as input devices, output devices and combination input/output devices. Interface devices may receive input or provide output. More particularly, output devices may render information for external presentation. Input devices may accept information from external sources. Examples of interface devices include keyboards, mouse devices, trackballs, microphones, touch screens, printing devices, display screens, speakers, network interface cards, etc. Interface devices allow the computer system 2402 to exchange information and to communicate with external entities, such as users and other systems.

The data storage 2418 includes a computer readable and writeable nonvolatile, or non-transitory, data storage medium in which instructions are stored that define a program or other object that is executed by the processor 2410. The data storage 2418 also may include information that is recorded, on or in, the medium, and that is processed by the processor 2410 during execution of the program. More specifically, the information may be stored in one or more data structures specifically configured to conserve storage space or increase data exchange performance. The instructions may be persistently stored as encoded signals, and the instructions may cause the processor 2410 to perform any of the functions described herein. The medium may, for example, be optical disk, magnetic disk or flash memory, among others. In operation, the processor 2410 or some other controller causes data to be read from the nonvolatile recording medium into another memory, such as the memory 2412, that allows for faster access to the information by the processor 2410 than does the storage medium included in the data storage 2418. The memory may be located in the data storage 2418 or in the memory 2412, however, the processor 2410 manipulates the data within the memory, and then copies the data to the storage medium associated with the data storage 2418 after processing is completed. A variety of components may manage data movement between the storage medium and other memory elements and examples are not limited to particular data management components. Further, examples are not limited to a particular memory system or data storage system.

Although the computer system 2402 is shown by way of example as one type of computer system upon which various aspects and functions may be practiced, aspects and functions are not limited to being implemented on the computer system 2402 as shown in FIG. 24. Various aspects and functions may be practiced on one or more computers having a different architectures or components than that shown in FIG. 24. For instance, the computer system 2402 may include specially programmed, special-purpose hardware, such as an application-specific integrated circuit (ASIC) tailored to perform a particular operation disclosed herein. While another example may perform the same function using a grid of several general-purpose computing devices running MAC OS System X with Motorola PowerPC processors and several specialized computing devices running proprietary hardware and operating systems.

The computer system 2402 may be a computer system including an operating system that manages at least a portion of the hardware elements included in the computer system 2402. In some examples, a processor or controller, such as the processor 2410, executes an operating system. Examples of a particular operating system that may be executed include a Windows-based operating system, such as, Windows NT, Windows 2000 (Windows ME), Windows XP, Windows Vista or Windows 7 operating systems, available from the Microsoft Corporation, a MAC OS System X operating system available from Apple Computer, one of many Linux-based operating system distributions, for example, the Enterprise Linux operating system available from Red Hat Inc., a Solaris operating system available from Sun Microsystems, or a UNIX operating systems available from various sources. Many other operating systems may be used, and examples are not limited to any particular operating system.

The processor 2410 and operating system together define a computer platform for which application programs in high-level programming languages are written. These component applications may be executable, intermediate, bytecode or interpreted code which communicates over a communication network, for example, the Internet, using a communication protocol, for example, TCP/IP. Similarly, aspects may be implemented using an object-oriented programming language, such as .Net, SmallTalk, Java, C++, Ada, or C# (C-Sharp). Other object-oriented programming languages may also be used. Alternatively, functional, scripting, or logical programming languages may be used.

Additionally, various aspects and functions may be implemented in a non-programmed environment, for example, documents created in HTML, XML or other format that, when viewed in a window of a browser program, can render aspects of a graphical-user interface or perform other functions. Further, various examples may be implemented as programmed or non-programmed elements, or any combination thereof. For example, a web page may be implemented using HTML while a data object called from within the web page may be written in C++. Thus, the examples are not limited to a specific programming language and any suitable programming language could be used. Accordingly, the functional components disclosed herein may include a wide variety of elements, e.g. specialized hardware, executable code, data structures or objects, that are configured to perform the functions described herein.

In some examples, the components disclosed herein may read parameters that affect the functions performed by the components. These parameters may be physically stored in any form of suitable memory including volatile memory (such as RAM) or nonvolatile memory (such as a magnetic hard drive). In addition, the parameters may be logically stored in a propriety data structure (such as a database or file defined by a user mode application) or in a commonly shared data structure (such as an application registry that is defined by an operating system). In addition, some examples provide for both system and user interfaces that allow external entities to modify the parameters and thereby configure the behavior of the components.

### Transcription System Processes

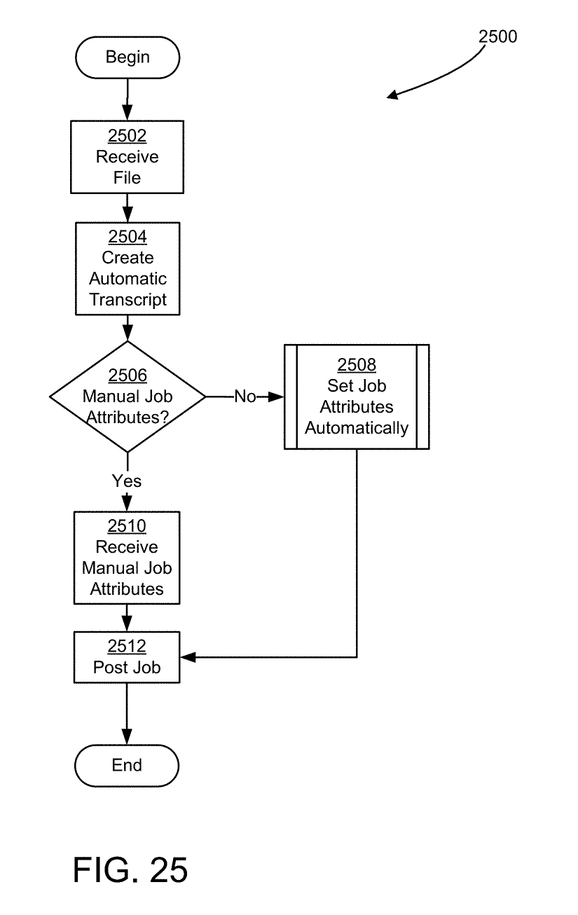

Some embodiments perform processes that add jobs to a transcription job market using a transcription system, such as the transcription system 2200 described above. One example of such a process is illustrated in FIG. 25. According to this example, a process 2500 includes acts of receiving a media file, creating an ASR transcription, receiving job attributes, setting job attributes automatically and posting a job.

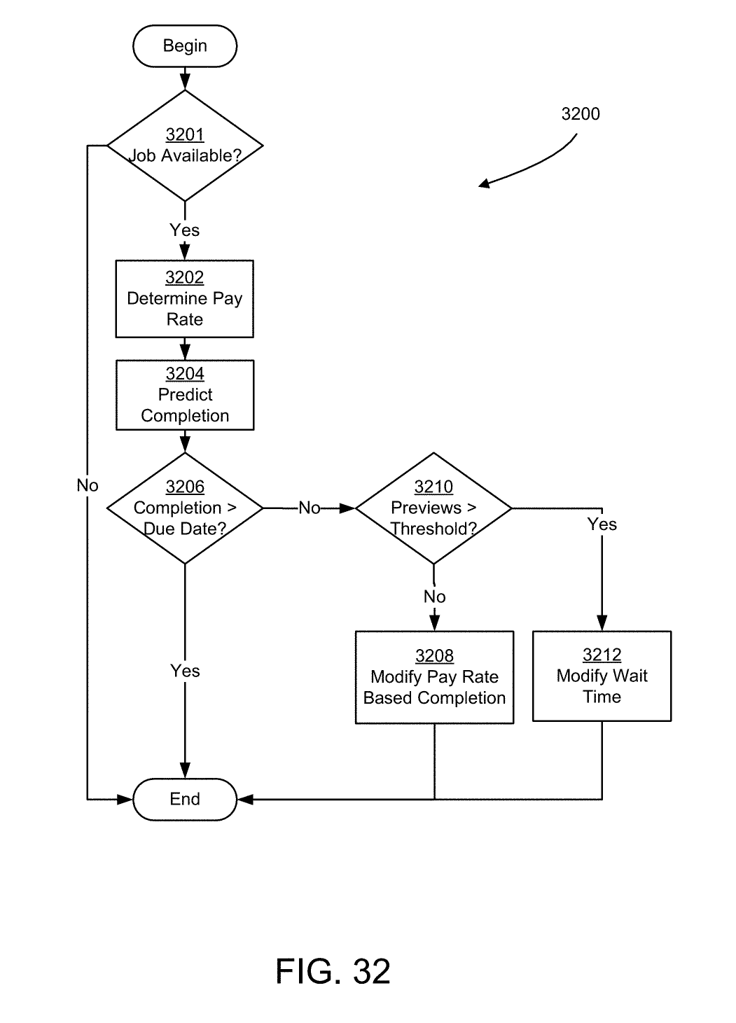

In act 2502, the transcription system receives a media file including content to be transcribed. Next, in act 2504, the transcription system uses an ASR device to produce an automatic transcription and associated information. After the automatic transcription is created, the transcription system optionally delivers the automatic transcription to the customer and determines whether attributes for a job to be associated with the media file will be set manually in act 2506. If so, the transcription system receives the manually entered job attributes in act 2510. Otherwise, the transcription system executes a process that sets the job attributes automatically in act 2508. This process is described further below with reference to FIG. 32. Once the job attributes have been set, the transcription system posts the job in act 2512, and the process 2500 ends.

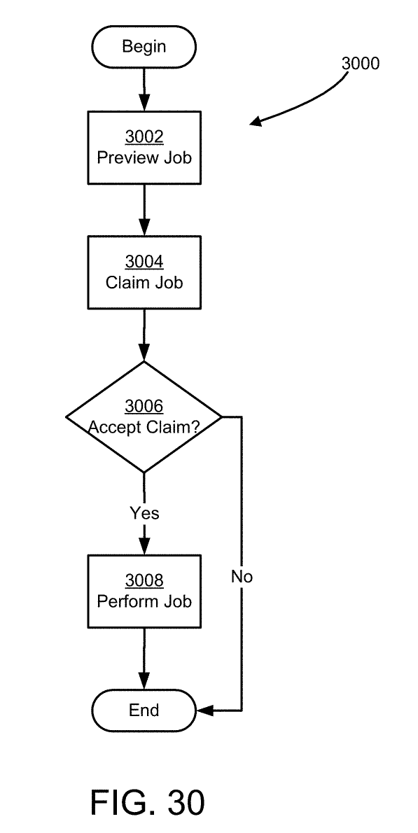

Other embodiments perform processes that allow and editor to perform a job listed on the transcription job market using a transcription system, such as the transcription system 2200 described above. One example of such a process is illustrated in FIG. 30. According to this example, a process 3000 includes acts of previewing a job, claiming a job and completing a job.

In act 3002, the transcription system receives a request to provide a preview of a job. In response to this request, the transcription system provides a preview of the job. The preview includes a preview of the content included in the media file associated with the job and draft transcription information for an ASR generated transcription that is associated with the media file. The preview may also include job attributes such as pay rate, domain, duration, and difficulty.

Next, in act 3004, the transcription system receives a request to claim the job. In response to this request, the transcription system determines whether to accept the claim using the processes disclosed herein. If the claim is not accepted, the process 3000 ends. If the claim is accepted, the process 3000 executes act 3008.

In the act 3008, the transcription system receives a request to perform the job. In response to this request, the transcription system provides a user interface and tools that enable an editor to perform work. While the editor is performing the work, the transcription system monitors progress and periodically saves work in process. Upon receipt of an indication that the editor has completed the job, the transcription system saves the completed job, and the process 3000 ends.

Other embodiments perform processes that monitor jobs to ensure the jobs are completed according to schedule using a transcription system, such as the transcription system 2200 described above. One example of such a process is illustrated in FIG. 31. According to this example, a process 3100 includes several acts that are described further below.

In act 3102, the transcription system determines whether a job should be assessed for attribute adjustment. The transcription system may make this determination based on a variety of factors including receipt of a request to assess the job from a component of the system or an entity external to the system (e.g., a request for immediate delivery of the job's output) or expiration of a predetermined period of time since the job was previously assessed, i.e., a wait time. If the job should not be assessed, the process 3100 ends. Otherwise, the process 3100 executes act 3104.

In the act 3104, the transcription system determines whether the job is assigned. If so, the transcription system executes act 3124. Otherwise, the transcription system determines whether the job is in progress in act 3106. If not, the transcription system executes act 3126. Otherwise, the transcription system executes the act 3128.

In the acts 3124, 3126 and 3128, the transcription system predicts the completion date and time of the job using one or more of the following factors: the current date and time, the amount of progress already complete for the job; historical productivity of the editor (in general or, more specifically, when editing media files having a characteristic in common with the media file associated with the job); the number of jobs currently claimed by the editor; the number of jobs the editor has in progress; and the due dates and times of the jobs claimed by the editor.

In some embodiments, the following equation is used to predict the completion date and time of the job:

Tc=To+[(1−Pj)*Dj*Xe]+[K1*Fc*Dc*Xc]+[K2*Fp*Dp*Xp]

Where,
Tc is the predicted completion time of the job
To is the current time
Pj is the progress on the job, expressed as a decimal fraction
Xe is the times-real-time-statistic for the editor, either the general statistic or the conditional statistic as determined by the job characteristics
Xc is the times-real-time-statistic for the editor, either the general statistic or the conditional statistic as determined by the claimed job characteristics, taken as a whole
Xp is the times-real-time-statistic for the editor, either the general statistic or the conditional statistic as determined by the in-progress job characteristics, taken as a whole
Dj is the duration of the job
Dc is the duration of the claimed but not yet in-progress jobs
Dp is the duration of the in-progress jobs

Fc is the fraction of the total claimed job duration accounted for by jobs which have a due date and time earlier than that of the current job
Fp is the fraction of the total in-progress jobs duration accounted for by jobs which have a due date and time earlier than the current job
K1 and K2 are tunable constants

In act 3108, the transcription system determines whether the predicted completion date and time of the job is before the due date and time of the job. If so, the process 3100 ends. Otherwise, the transcription system executes act 3118.

In act 3110, the transcription system determines whether the predicted completion date and time of the job is before the due date and time of the job. If so, the process 3100 ends. Otherwise, the transcription system executes a process that sets the job attributes automatically in act 3120. This process is described further below with reference to FIG. 32. Once the job attributes have been set, the process 3100 ends.

In act 3114, the transcription system determines whether the predicted completion date and time of the job is before the due date and time of the job. If so, the process 3100 ends. Otherwise, the transcription system determines whether to revoke the job in act 3112. If not, the process 3100 ends. Otherwise, the transcription system revokes the job in act 3116.

In act 3118, the transcription system determines whether to split the job. If not, the process 3100 ends. Otherwise, the transcription system splits the job in act 3122, and the process 3100 ends.

As discussed above with reference to FIGS. 25 and 31, some embodiments perform processes that set attributes of jobs using a transcription system, such as the transcription system 2200 described above. One example of such a process is illustrated in FIG. 32. According to this example, a process 3200 includes several acts that are described further below.

In act 3201, the transcription system determines if the job is available. In not, the process 3200 ends. Otherwise, the transcription system determines a pay rate for the job in act 3202. The transcription system may make this determination based on any of a variety of factors including due date and time, difficulty, domain and ASR_cost.

In act 3204, the transcription system predicts a completion date and time for the job for each editor. The transcription system may make this determination based on any of a variety of factors including difficulty, domain and historical XRT of previously completed, similar jobs.

In act 3206, the transcription system determines whether the completion date and time is prior to the due date and time for the job. If so, the process 3200 ends. Otherwise, the transcription system determines whether the number of previews provided for the job transgresses a threshold in act 3210. If not, the transcription system executes act 3208. Otherwise, the transcription system executes act 3212.

In act 3208, the transcription system modifies the pay rate based on the difference between the due date and time to the completion date and time, and the process 3200 ends. For instance, the transcription system may set the modified pay rate equal to the unmodified pay rate plus a date and time increment amount multiplied by the difference between the due date and time and the completion date and time.

In act 3212, the transcription system modifies the wait time for reassessment of the job, and the process 3200 ends. For instance, the transcription system may set the modified wait time equal to the unmodified wait time plus an increment amount.

Having thus described several aspects of at least one example, it is to be appreciated that various alterations, modifications, and improvements will readily occur to those skilled in the art. For instance, examples disclosed herein may also be used in other contexts. Such alterations, modifications, and improvements are intended to be part of this disclosure, and are intended to be within the scope of the examples discussed herein. Accordingly, the foregoing description and drawings are by way of example only.

1. A mobile computing device implementing a mobile recording application, the mobile computing device comprising:
a memory;
a microphone;
a network interface; and
at least one processor coupled to the memory, the microphone, and the network interface and configured to
record, via the microphone, at least one media file comprising content divisible into a plurality of sections;
associate a first portion of the at least one media file with a first section of the plurality of sections;
associate a second portion of the at least one media file with a second section of the plurality of sections;
generate transcription request information specifying that the first portion be transcribed without human review and that the second portion be transcribed with human review; and
transmit, via the network interface, the at least one media file and the transcription request information to a transcription system distinct from the mobile computing device.

2. The mobile computing device of claim 1, wherein the content is descriptive of a patient encounter to be documented in an electronic health record (EHR) of the patient and the plurality of sections comprise EHR sections.

3. The mobile computing device of claim 1, wherein the at least one processor is configured to associate the first portion of the at least one media file with the first section in response to identifying a keyword within the first portion, the keyword being associated with the first section.

4. The mobile computing device of claim 1, further comprising a display configured to present at least one control associated with the first section, wherein the at least one processor is coupled to the display and configured to associate the first portion of the at least one media file with the first section in response to receiving a selection of the at least one control prior to recording the first portion.

5. The mobile computing device of claim 1, further comprising a display configured to present a plurality of controls comprising a first control associated with the first section and a second control associated with the second section, wherein the at least one processor is configured to generate the transcription request information at least in part by identifying that the first control is deselected and identifying that the second control is selected.

6. The mobile computing device of claim 5, wherein the at least one processor is further configured to deselect the first control and select the second control in response to accessing information representative of a default set of sections.

7. The mobile computing device of claim 5, wherein the at least one processor is further configured to deselect the first control in response to a first selection received via the display.

8. The mobile computing device of claim 1, wherein the at least one processor is further configured to:
initiate generation an automatic speech recognition (ASR) transcript of at least the first portion of the at least one media file;
compare an indicator of confidence in the ASR transcript to a threshold confidence; and
select the first portion to be transcribed without human review in response to the indictor being greater than the threshold confidence.

9. The mobile computing device of claim 1, wherein the at least one processor is further configured to:
initiate generation an automatic speech recognition (ASR) transcript of at least the second portion of the at least one media file;
compare an indicator of confidence in the ASR transcript to a threshold confidence; and
select the second portion to be transcribed with human review in response to the indictor being less than the threshold confidence.

10. The mobile computing device of claim 9, wherein the at least one processor is configured to initiate generation of the ASR transcript by either initiating a local ASR process or transmitting a message to an ASR system distinct from the mobile computing device.

11. A transcript delivery system comprising:
a mobile computing device implementing a mobile recording application, the mobile computing device comprising
a memory;
a microphone;
a network interface; and
at least one processor coupled to the memory, the microphone, and the network interface and configured to
record, via the microphone, at least one media file comprising content divisible into a plurality of sections;
associate a first portion of the at least one media file with a first section of the plurality of sections;
associate a second portion of the at least one media file with a second section of the plurality of sections;
generate transcription request information specifying that the first portion be transcribed without human review and that the second portion be transcribed with human review; and
transmit, via the network interface, the at least one media file and the transcription request information to a transcription system distinct from the mobile computing device; and
the transcription system, wherein the transcription system is configured to
generate a final transcript of the at least one media file in response to receiving the at least one media file and the transcription request information; and
transmit the final transcript to a database system distinct from the transcript delivery system.

12. The transcript delivery system of claim 11, wherein the content is descriptive of a patient encounter to be documented in an electronic health record (EHR) of the patient, the plurality of sections comprise EHR sections, and the final transcript is divided into the EHR sections.

13. A method of efficiently transcribing content divisible into a plurality of sections using a computer system comprising a mobile computing device, the method comprising:
recording, via a microphone of the mobile computing device, at least one media file comprising the content;
associating a first portion of the at least one media file with a first section of the plurality of sections;
associating a second portion of the at least one media file with a second section of the plurality of sections;
generating transcription request information specifying that the first portion be transcribed without human review and that the second portion be transcribed with human review; and
transmitting, via a network interface of the mobile computing device, the at least one media file and the transcription request information to a transcription system distinct from the mobile computing device.

14. The method of claim 13, wherein recording the at least one media file comprises recording content descriptive of a patient encounter to be documented in an electronic health record (EHR) of the patient, the content being divisible into EHR sections.

15. The method of claim 13, wherein associating the first portion of the at least one media file with the first section comprises identifying a keyword within the first portion, the keyword being associated with the first section.

16. The method of claim 13, further comprising presenting, via a display of the mobile computing device, at least one control associated with the first section, wherein associating the first portion of the at least one media file with the first section comprises receiving a selection of the at least one control prior to recording the first portion.

17. The method of claim 13, further comprising presenting, via a display of the mobile computing device, a plurality of controls comprising a first control associated with the first section and a second control associated with the second section, wherein generating the transcription request information comprises identifying that the first control is deselected and identifying that the second control is selected.

18. The method of claim 17, further comprising deselecting the first control and selecting the second control in response to accessing information representative of a default set of sections.

19. The method of claim 17, further comprising deselecting the first control in response to a first selection received via the display.

20. The method of claim 13, further comprising:
initiating generation an automatic speech recognition (ASR) transcript of at least the first portion of the at least one media file;
comparing an indicator of confidence in the ASR transcript to a threshold confidence; and
selecting the first portion to be transcribed without human review in response to the indictor being greater than the threshold confidence.

21. The method of claim 13, further comprising:
initiating generation an automatic speech recognition (ASR) transcript of at least the second portion of the at least one media file;
comparing an indicator of confidence in the ASR transcript to a threshold confidence; and
selecting the second portion to be transcribed with human review in response to the indictor being less than the threshold confidence.

22. The method of claim 21, wherein initiating generation of the ASR transcript comprises either initiating a local ASR process or transmitting a message to an ASR system distinct from the mobile computing device.

23. The method of claim 13, further comprising:
generating, by a transcription system distinct from the mobile computing device, a final transcript of the at least one media file in response to receiving the at least one media file and the transcription request information; and
transmitting the final transcript to a database system distinct from the transcript delivery system.

24. The method of claim 23, wherein generating the final transcript comprises generating a final transcript of a patient encounter to be documented in an electronic health record (EHR) of the patient, the final transcript being divided into EHR sections.

25. A non-transitory computer readable medium storing sequences of computer executable instructions for efficiently transcribing content divisible into a plurality of sections, the sequences of computer executable instructions comprising instructions that instruct at least one processor to:
recording, via a microphone of the mobile computing device, at least one media file comprising the content;
associating a first portion of the at least one media file with a first section of the plurality of sections;
associating a second portion of the at least one media file with a second section of the plurality of sections;
generating transcription request information specifying that the first portion be transcribed without human review and that the second portion be transcribed with human review; and
transmitting, via a network interface of the mobile computing device, the at least one media file and the transcription request information to a transcription system distinct from the mobile computing device.

26. The computer readable medium of claim 25, wherein recording the at least one media file comprises recording content descriptive of a patient encounter to be documented in an electronic health record (EHR) of the patient, the content being divisible into EHR sections.

27. A system comprising:
a mobile computing device implementing a mobile application, the mobile computing device comprising
a memory;
a microphone;
a network interface; and
at least one processor coupled to the memory, the microphone, and the network interface and configured to
record, via the microphone, audio comprises a plurality of electronic health record (EHR) sections;
identify a first EHR section of the plurality of EHR sections within the audio;
identify a second EHR section of the plurality of EHR sections within the audio;
generate an order specifying that the first EHR section be transcribed via automatic speech recognition only and that the second EHR section be reviewed by a professional transcription editor; and
transmit the audio and the order to a transcription system distinct from the mobile computing device; and
the transcription system, wherein the transcription system is configured to
generate a final transcript of the audio in response to receiving the audio and order; and
post the final transcript to an EHR system distinct from the mobile computing device and the transcription system.
